(19) United States
(12) Patent Application Publication
(10) Pub. No.: US 2021/0277409 A1
(43) Pub. Date: Sep. 9, 2021

(54) METHODS FOR SELECTING TRANSFORMED PLANTS

(71) Applicant: PIONEER HI-BRED INTERNATIONAL, INC., JOHNSTON, IA (US)

(72) Inventors: AJITH ANAND, WEST DES MOINES, IA (US); HYEON-JE CHO, ANKENY, IA (US); THEODORE M KLEIN, WILMINGTON, DE (US)

(73) Assignee: PIONEER HI-BRED INTERNATIONAL, INC., JOHNSTON, IA (US)

(21) Appl. No.: 17/255,132

(22) PCT Filed: Jun. 25, 2019
(86) PCT No.: PCT/US19/38972

§ 371 Date: Dec. 22, 2020

Related U.S. Application Data
(60) Provisional application No. 62/691,120, filed on Jun. 28, 2018.
(60) Provisional application No. 62/723,097, filed on Aug. 27, 2018.

(51) Int. Cl. C12N 15/82

(52) U.S. Cl. CPC C12N 15/8209

(57) ABSTRACT

Spectinomycin resistant or streptomycin resistant transgenic plants and methods of making such plants are provided.

### FIELD OF THE DISCLOSURE

The present disclosure relates generally to the field of plant molecular biology, including genetic manipulation of plants. More specifically, the present disclosure pertains to spectinomycin resistant or streptomycin resistant transgenic plants and methods of making and selecting such plants.

### CROSS-REFERENCE TO RELATED APPLICATIONS

This application claims the benefit of PCT Application Serial Number PCT/US2019/038972, filed Jun. 25, 2019, which claims the benefit of U.S. Provisional Application No. 62/691,120, filed Jun. 28, 2018 and U.S. Provisional Application No. 62/723,097, filed Aug. 27, 2018, which are hereby incorporated herein in their entireties by reference.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 7785-US-PCT_ST25, created on Dec. 19, 2020, and having a size of 775,607 bytes and is filed concurrently with the specification. The sequence listing contained in this ASCII formatted document is part of the specification and is herein incorporated by reference in its entirety.

### BACKGROUND

The use of standard transformation and regeneration protocols is time consuming and inefficient, and negatively impacts transgenic product development timelines, given that there is usually a seasonally limited “priority development window” for making decisions regarding which genetic constructs to prioritize for use in larger scale field work based on results obtained during initial research. The available standard methods of transformation and regeneration have multiple drawbacks that limit the speed and efficiency with which transgenic plants can be produced and screened. For example, many standard methods of transformation and regeneration require the use of high auxin or cytokinin levels and require steps involving either embryogenic callus formation or organogenesis, leading to procedures that take many weeks before producing plants for growth in a greenhouse setting following transformation. These methods can take 12-23 weeks to produce plants, which include the steps of supplying 2,4-D (auxin) to stimulate somatic embryo formation in corn (taking up to 8 weeks), production of embryogenic callus from the primary somatic embryos (taking up to an additional 8 weeks), forming shoots (taking up to an additional 3 weeks), and finally rooting (taking up to an additional 1 to 3 weeks). Other methods immediately supply a cytokinin along with the auxin to stimulate direct morphogenesis to produce shoots and direct plant formation in from 8 to 28 weeks after transformation. There remains a need for transformation methods that produce significantly higher transformation frequencies and significantly more quality events (events containing one copy of a trait gene cassette with no vector (plasmid) backbone) in multiple inbred lines using a variety of starting tissue types, including transformed inbreds representing a range of genetic diversities and having significant commercial utility.

### SUMMARY

The present disclosure comprises methods and compositions for producing transgenic plants that are spectinomycin resistant or streptomycin resistant. Methods of making and selecting such plants, and the transgenic plants so make are also provided.

In an aspect, the disclosure provides a plant transformed with a recombinant expression cassette comprising a marker gene cassette comprising a DNA sequence imparting spectinomycin resistance or streptomycin resistance in plants, wherein the DNA sequence comprises a nucleotide sequence selected from the group consisting of (a) at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants; (b) a nucleotide sequence that is at least 95% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants; (c) a nucleotide sequence that is at least 70% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants; (d) a nucleotide sequence encoding a polypeptide of at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; (e) a nucleotide sequence encoding a polypeptide that is at least 95% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; and (f) a nucleotide sequence encoding a polypeptide that is at least 70% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants. In a further aspect, the recombinant expression cassette further comprises a trait gene cassette comprising a heterologous nucleotide sequence of interest. In a further aspect, the heterologous nucleotide sequence of interest comprises a trait gene encoding a gene product conferring nutritional enhancement, pest resistance, herbicide resistance, abiotic stress tolerance, increased yield, drought resistance, cold tolerance, pathogen resistance, insect resistance, nitrogen use efficiency (NUE), disease resistance, or an ability to alter a metabolic pathway. In a further aspect, the recombinant expression cassette further comprises a morphogenic gene cassette comprising a morphogenic gene. In a further aspect, the morphogenic gene comprises: (i) a nucleotide sequence encoding a WUS/WOX homeobox polypeptide; or (ii) a nucleotide sequence encoding a Babyboom (BBM) polypeptide or an Ovule Development Protein 2 (ODP2) polypeptide; or (iii) a combination of (i) and (ii). In a further aspect, the morphogenic gene comprises the nucleotide sequence encoding the WUS/WOX homeobox polypeptide. In a further aspect, the nucleotide sequence encoding the WUS/WOX homeobox polypeptide is selected from WUS1, WUS2, WUS3, WOX2A, WOX4, WOX5, and WOX9. In a further aspect, the morphogenic gene comprises a nucleotide sequence encoding the Babyboom (BBM) polypeptide or the Ovule Development Protein 2 (ODP2) polypeptide. In a further aspect, the nucleotide sequence encoding the Babyboom (BBM) polypeptide is selected from BBM2, BMN2, and BMN3 or the Ovule Development Protein 2 (ODP2) polypeptide is ODP2. In a further aspect, the morphogenic gene comprises a nucleotide sequence encoding the WUS/WOX homeobox polypeptide and the Babyboom (BBM) polypeptide or the Ovule Development Protein 2 (ODP2) polypeptide. In a further aspect, the nucleotide sequence encoding the WUS/WOX homeobox polypeptide is selected from WUS1, WUS2, WUS3, WOX2A, WOX4, WOX5, and WOX9 and the Babyboom (BBM) polypeptide is selected from BBM2, BMN2, and BMN3 or the Ovule Development Protein 2 (ODP2) polypeptide is ODP2. In a further aspect, the recombinant expression cassette further comprises a site-specific recombinase cassette comprising a nucleotide sequence encoding a site-specific recombinase selected from FLP, Cre, SSV1, lambda Int, phi C31 Int, HK022, R, Gin, Tn1721, CinH, ParA, Tn5053, Bxbl, TP907-1, or U153. In a further aspect, the site-specific recombinase is operably linked to a constitutive promoter, an inducible promoter, or a developmentally regulated promoter. In a further aspect, the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2); the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and the developmentally regulated promoter is selected from PLTP, PLTP1, PLTP2, PLTP3, SDR, LGL, LEA-14A, or LEA-D34. In a further aspect, the morphogenic gene is operably linked to a constitutive promoter, an inducible promoter, or a developmentally regulated promoter. In a further aspect, the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2); the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and the developmentally regulated promoter is selected from PLTP, PLTP1, PLTP2, PLTP3, SDR, LGL, LEA-14A, or LEA-D34. In a further aspect, the plant is a monocot or a dicot. In a further aspect, the seed of the transformed plant comprises the trait gene cassette and not the marker gene cassette. In a further aspect, the seed of the transformed plant comprises the trait gene cassette and not the marker gene cassette or the morphogenic gene cassette. In a further aspect, the seed of the transformed plant comprises the trait gene cassette and not the marker gene cassette or the morphogenic gene cassette or the site-specific recombinase cassette.

In an aspect, the disclosure provides a method of producing a transgenic plant expressing a trait gene cassette comprising: transforming a plant cell with a recombinant expression cassette comprising a trait gene cassette and a marker gene cassette, the marker gene cassette comprising a DNA sequence imparting spectinomycin resistance or streptomycin resistance in plants, wherein the DNA sequence comprises a nucleotide sequence selected from the group consisting of: (a) at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants; (b) a nucleotide sequence that is at least 95% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants; (c) a nucleotide sequence that is at least 70% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants; (d) a nucleotide sequence encoding a polypeptide of at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; (e) a nucleotide sequence encoding a polypeptide that is at least 95% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; and (f) a nucleotide sequence encoding a polypeptide that is at least 70% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; selecting a spectinomycin resistant or a streptomycin resistant transgenic plant cell; and regenerating the transgenic plant expressing the trait gene cassette. In a further aspect, the trait gene cassette comprises a heterologous nucleotide sequence of interest. In a further aspect, the heterologous nucleotide sequence of interest comprises a trait gene encoding a gene product conferring nutritional enhancement, pest resistance, herbicide resistance, abiotic stress tolerance, increased yield, drought resistance, cold tolerance, pathogen resistance, insect resistance, nitrogen use efficiency (NUE), disease resistance, or an ability to alter a metabolic pathway. In a further aspect, the recombinant expression cassette further comprises a morphogenic gene cassette. In a further aspect, the morphogenic gene cassette comprises a morphogenic gene. In a further aspect, the morphogenic gene comprises: (i) a nucleotide sequence encoding a WUS/WOX homeobox polypeptide; or (ii) a nucleotide sequence encoding a Babyboom (BBM) polypeptide or an Ovule Development Protein 2 (ODP2) polypeptide; or (iii) a combination of (i) and (ii). In a further aspect, the morphogenic gene comprises a nucleotide sequence encoding the WUS/WOX homeobox polypeptide. In a further aspect, the nucleotide sequence encoding the WUS/WOX homeobox polypeptide is selected from WUS1, WUS2, WUS3, WOX2A, WOX4, WOX5, and WOX9. In a further aspect, the morphogenic gene comprises a nucleotide sequence encoding the Babyboom (BBM) polypeptide or the Ovule Development Protein 2 (ODP2) polypeptide. In a further aspect, the nucleotide sequence encoding the Babyboom (BBM) polypeptide is selected from BBM2, BMN2, and BMN3 or the Ovule Development Protein 2 (ODP2) polypeptide is ODP2. In a further aspect, the morphogenic gene comprises a nucleotide sequence encoding the WUS/WOX homeobox polypeptide and the Babyboom (BBM) polypeptide or the Ovule Development Protein 2 (ODP2) polypeptide. In a further aspect, the nucleotide sequence encoding the WUS/WOX homeobox polypeptide is selected from WUS1, WUS2, WUS3, WOX2A, WOX4, WOX5, and WOX9 and the Babyboom (BBM) polypeptide is selected from BBM2, BMN2, and BMN3 or the Ovule Development Protein 2 (ODP2) polypeptide is ODP2. In a further aspect, the recombinant expression cassette further comprises a site-specific recombinase cassette. In a further aspect, the site-specific recombinase cassette comprises a nucleotide sequence encoding a site-specific recombinase selected from FLP, Cre, SSV1, lambda Int, phi C31 Int, HK022, R, Gin, Tn1721, CinH, ParA, Tn5053, Bxbl, TP907-1, or U153. In a further aspect, the nucleotide sequence encoding the site-specific recombinase is operably linked to a constitutive promoter, an inducible promoter, or a developmentally regulated promoter. In a further aspect, the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2); the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and the developmentally regulated promoter is selected from PLTP, PLTP1, PLTP2, PLTP3, SDR, LGL, LEA-14A, or LEA-D34. In a further aspect, the morphogenic gene is operably linked to a constitutive promoter, an inducible promoter, or a developmentally regulated promoter. In a further aspect, the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2); the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and the developmentally regulated promoter is selected from PLTP, PLTP1, PLTP2, PLTP3, SDR, LGL, LEA-14A, or LEA-D34. In a further aspect, the method further comprising excising or segregating away the marker gene cassette from the transgenic plant expressing the trait gene cassette. In a further aspect, the method further comprising excising or segregating away the marker gene cassette and the morphogenic gene cassette from the transgenic plant expressing the trait gene cassette. In a further aspect, the method further comprising excising or segregating away the marker gene cassette, the morphogenic gene cassette, and the site-specific recombinase cassette from the transgenic plant expressing the trait gene cassette. In a further aspect, the plant cell is a monocot or a dicot. In a further aspect, a seed from the transgenic plant produced by the method disclosed herein. In a further aspect, a seed of the transgenic plant produced by the method disclosed herein, wherein the seed comprises the trait gene cassette and not the marker gene cassette. In a further aspect, a seed of the transgenic plant produced by the method disclosed herein, wherein the seed comprises the trait gene cassette and not the marker gene cassette or the morphogenic gene cassette. In a further aspect, a seed of the transgenic plant produced by the method disclosed herein, wherein the seed comprises the trait gene cassette and not the marker gene cassette or the morphogenic gene cassette or the site-specific recombinase cassette. In a further aspect, the recombinant expression cassette resides in a disarmed Agrobacteria, an Ochrobactrum bacteria or a Rhizobiaceae bacteria. In a further aspect, the disarmed Agrobacteria is selected from the group of AGL-1, EHA105, GV3101, LBA4404, and LBA4404 THY-. In a further aspect, the Ochrobactrum bacteria is selected from Table 2. In a further aspect, the Rhizobiaceae bacteria is selected from Table 3.

### DETAILED DESCRIPTION

The disclosures herein will be described more fully hereinafter. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods pertain having the benefit of the teachings presented in the following descriptions and the associated figures. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

The terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed methods belong. In this specification and in the claims which follow, reference will be made to a number of terms which shall be defined herein.

The present disclosure comprises methods for producing a transgenic plant using a morphogenic gene. As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. As used herein, the term “morphogenic factor” means a morphogenic gene and/or the protein expressed by a morphogenic gene.

A morphogenic gene is involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof, such as WUS/WOX genes (WUS1, WUS2, WUS3, WOX2A, WOX4, WOX5, or WOX9) see, U.S. Pat. Nos. 7,348,468 and 7,256,322 and US Patent Application Publication Numbers 2017/0121722 and 2007/0271628, herein incorporated by reference in their entirety; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39. Modulation of WUS/WOX is expected to modulate plant and/or plant tissue phenotype including plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

Morphogenic polynucleotide sequences and amino acid sequences of WUS/WOX homeobox polypeptides are useful in the disclosed methods. The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In an aspect, the WUS/WOX homeobox polypeptide useful in the methods of the disclosure is a WUS1, WUS2, WUS3, WOX2A, WOX4, WOX5, WOX5A, or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The WUS/WOX homeobox polypeptide useful in the methods of the disclosure can be obtained from or derived from any of the plants described herein. Additional WUS/WOX genes useful in the present disclosure include, but are not limited to those disclosed in Table 1. The encoded WUS/WOX polypeptides are also listed in Table 1.

TABLE 1

| SEQ | Polynucleotide (DNA) | | |
|---|---|---|---|
| ID NO. | or Polypeptide (PRT) | Name | Description |
| 46 | DNA | AT-WUS | Arabidopsis thaliana WUS coding |
| | | | sequence |
| 47 | PRT | AT-WUS | Arabidopsis thaliana WUS protein |
| | | | sequence |
| 48 | DNA | LJ-WUS | Lotus japonicus WUS coding sequence |
| 49 | PRT | LJ-WUS | Lotus japonicus WUS protein sequence |
| 50 | DNA | GM-WUS | Glycine max WUS coding sequence |
| 51 | PRT | GM-WUS | Glycine max WUS protein sequence |
| 52 | DNA | CS-WUS | Camelina sativa WUS coding sequence |
| 53 | PRT | CS-WUS | Camelina sativa WUS protein sequence |
| 54 | DNA | CR-WUS | Capsella rubella WUS coding sequence |
| 55 | PRT | CR-WUS | Capsella rubella WUS protein sequence |
| 56 | DNA | AA-WUS | Arabis alpina WUS coding sequence |
| 57 | PRT | AA-WUS | Arabis alpina WUS protein sequence |
| 58 | DNA | RS-WUS | Raphanus sativus WUS coding sequence |
| 59 | PRT | RS-WUS | Raphanus sativus WUS protein sequence |
| 60 | DNA | BN-WUS | Brassica napus WUS coding sequence |
| 61 | PRT | BN-WUS | Brassica napus WUS protein sequence |
| 62 | DNA | BO-WUS | Brassica oleracea var. oleracea WUS |
| | | | coding sequence |
| 63 | PRT | BO-WUS | Brassica oleracea var. oleracea WUS |
| | | | protein sequence |
| 64 | DNA | HA-WUS | Helianthus annuus WUS coding |
| | | | sequence |
| 65 | PRT | HA-WUS | Helianthus annuus WUS protein |
| | | | sequence |
| 66 | DNA | PT-WUS | Populus trichocarpa WUS coding |
| | | | sequence |
| 67 | PRT | PT-WUS | Populus trichocarpa WUS protein |
| | | | sequence |
| 68 | DNA | VV-WUS | Vitis vinifera WUS coding sequence |
| 69 | PRT | VV-WUS | Vitis vinifera WUS protein sequence |
| 70 | DNA | AT-WUS | Arabidopsis thaliana WUS coding |
| | | | sequence (soy optimized) |
| 71 | PRT | AT-WUS | Arabidopsis thaliana WUS protein |
| | | | sequence |
| 72 | DNA | LJ-WUS | Lotus japonicus WUS coding sequence |
| | | | (soy optimized) |
| 73 | PRT | LJ-WUS | Lotus japonicus WUS protein sequence |
| 74 | DNA | MT-WUS | Medicago trunculata WUS coding |
| | | | sequence (soy optimized) |
| 75 | PRT | MT-WUS | Medicago trunculata WUS protein |
| | | | sequence |
| 76 | DNA | PH-WUS | Petunia hybrida WUS coding sequence |
| | | | (soy optimized) |
| 77 | PRT | PH-WUS | Petunia hybrida WUS protein sequence |
| 78 | DNA | PV-WUS | Phaseolus vulgaris WUS coding |
| | | | sequence (soy optimized) |
| 79 | PRT | PV-WUS | Phaseolus vulgaris WUS protein |
| | | | sequence |
| 80 | DNA | ZM-WUS1 | Zea mays WUS1 coding sequence |
| 81 | PRT | ZM-WUS1 | Zea mays WUS1 protein sequence |
| 82 | DNA | ZM-WUS2 | Zea mays WUS2 coding sequence |
| 83 | PRT | ZM-WUS2 | Zea mays WUS2 protein sequence |
| 84 | DNA | ZM-WUS3 | Zea mays WUS3 coding sequence |
| 85 | PRT | ZM-WUS3 | Zea mays WUS3 protein sequence |
| 86 | DNA | ZM-WOX2A | Zea mays WOX2A coding sequence |
| 87 | PRT | ZM-WOX2A | Zea mays WOX2A protein sequence |
| 88 | DNA | ZM-WOX4 | Zea mays WOX4 coding sequence |
| 89 | PRT | ZM-WOX4 | Zea mays WOX4 protein sequence |
| 90 | DNA | ZM-WOX5A | Zea mays WOX5A coding sequence |
| 91 | PRT | ZM-WOX5A | Zea mays WOX5A protein sequence |
| 92 | DNA | ZM-WOX9 | Zea mays WOX9 coding sequence |
| 93 | PRT | ZM-WOX9 | Zea mays WOX9 protein sequence |
| 94 | DNA | GG- WUS | Gnetum gnemon WUS coding sequence |
| 95 | PRT | GG- WUS | Gnetum gnemon WUS protein sequence |
| 96 | DNA | MD-WUS | Malus domestica WUS coding sequence |
| 97 | PRT | MD-WUS | Malus domestica WUS protein sequence |
| 98 | DNA | ME-WUS | Manihot esculenta WUS coding sequence |
| 99 | PRT | ME-WUS | Manihot esculenta WUS protein |
| | | | sequence |
| 100 | DNA | KF-WUS | Kalanchoe fedtschenkoi WUS coding |
| | | | sequence |
| 101 | PRT | KF-WUS | Kalanchoe fedtschenkoi WUS protein |
| | | | sequence |
| 102 | DNA | GH-WUS | Gossypium hirsutum WUS coding |
| | | | sequence |
| 103 | PRT | GH-WUS | Gossypium hirsutum WUS protein |
| | | | sequence |
| 104 | DNA | ZOSMA-WUS | Zostera marina WUS coding sequence |
| 105 | PRT | ZOSMA-WUS | Zostera marina WUS protein sequence |
| 106 | DNA | AMBTR-WUS | Amborella trichopoda WUS coding |
| | | | sequence |
| 107 | PRT | AMBTR-WUS | Amborella trichopoda WUS protein |
| | | | sequence |
| 108 | DNA | AC-WUS | Aquilegia coerulea WUS coding |
| | | | sequence |
| 109 | PRT | AC-WUS | Aquilegia coerulea WUS protein |
| | | | sequence |
| 110 | DNA | AH-WUS | Amaranthus hypochondriacus WUS |
| | | | coding sequence |
| 111 | PRT | AH-WUS | Amaranthus hypochondriacus WUS |
| | | | protein sequence |
| 112 | DNA | CUCSA-WUS | Cucumis sativus WUS coding sequence |
| 113 | PRT | CUCSA -WUS | Cucumis sativus WUS protein sequence |
| 114 | DNA | PINTA-WUS | Pinus taeda WUS coding sequence |
| 115 | PRT | PINTA-WUS | Pinus taeda WUS protein sequence |

Other morphogenic genes useful in the present disclosure include, but are not limited to, LEC1 (U.S. Pat. No. 6,825,397 incorporated herein by reference in its entirety, Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabiopsis AGL15 gene (Harding et al., 2003, Plant Physiol. 133:653-663), the FUSCA gene (Castle and Meinke, Plant Cell 6:25-41), and the PICKLE gene (Ogas et al., 1999, PNAS 96:13839-13844). Any of these morphogenic genes may also be combined with any of the WUS/WOX genes described herein.

As used herein, the term “transcription factor” means a protein that controls the rate of transcription of specific genes by binding to the DNA sequence of the promoter and either up-regulating or down-regulating expression. Examples of transcription factors, which are also morphogenic genes, include members of the AP2/EREBP family (including the BBM (ODP2), plethora and aintegumenta sub-families, CAAT-box binding proteins such as LEC1 and HAP3, and members of the MYB, bHLH, NAC, MADS, bZIP and WRKY families.

Morphogenic polynucleotide sequences and amino acid sequences of Ovule Development Protein 2 (ODP2) polypeptides, and related polypeptides, e.g., Babyboom (BBM) protein family proteins are useful in the methods of the disclosure. In an aspect, a polypeptide comprising two AP2-DNA binding domains is an ODP2, BBM2, BMN2, or BMN3 polypeptide see, US Patent Application Publication Number 2017/0121722, herein incorporated by reference in its entirety. ODP2 polypeptides useful in the methods of the disclosure contain two predicted APETALA2 (AP2) domains and are members of the AP2 protein family (PFAM Accession PF00847). The AP2 family of putative transcription factors has been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins.

ODP2 polypeptides useful in the methods of the disclosure share homology with several polypeptides within the AP2 family, e.g., see FIG. 1 of U.S. Pat. No. 8,420,893, which is incorporated herein by reference in its entirety, provides an alignment of the maize and rice ODP2 polypeptides with eight other proteins having two AP2 domains. A consensus sequence of all proteins appearing in the alignment of U.S. Pat. No. 8,420,893 is also provided in FIG. 1 therein. The polypeptide comprising the two AP2-DNA binding domains useful in the methods of the disclosure can be obtained from or derived from any of the plants described herein. In an aspect, the polypeptide comprising the two AP2-DNA binding domains useful in the methods of the disclosure is an ODP2 polypeptide. In an aspect, the polypeptide comprising the two AP2-DNA binding domains useful in the methods of the disclosure is a BBM2 polypeptide. The ODP2 polypeptide and the BBM2 polypeptide useful in the methods of the disclosure can be obtained from or derived from any of the plants described herein.

A morphogenic gene may be stably incorporated into the genome of a plant or it may be transiently expressed. In an aspect, expression of the morphogenic gene is controlled. The controlled expression may be a pulsed expression of the morphogenic gene for a particular period of time. Alternatively, the morphogenic gene may be expressed in only some transformed cells and not expressed in others. The control of expression of the morphogenic gene can be achieved by a variety of methods as disclosed herein below. The morphogenic genes useful in the methods of the disclosure may be obtained from or derived from any plant species described herein. Methods of regulating expression can be found in U.S. Pat. No. 9,765,352 incorporated herein by reference in its entirety.

The term “plant” refers to whole plants, plant organs (e.g., leaves, stems, roots, etc.), plant tissues, plant cells, plant parts, seeds, propagules, embryos and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature influorescences, tassel, immature ear, silks, cotyledons, immature cotyledons, embryonic axes, meristematic regions, callus tissue, cells from leaves, cells from stems, cells from roots, cells from shoots, gametophytes, sporophytes, pollen and microspores. Plant parts include differentiated and undifferentiated tissues including, but not limited to, roots, stems, shoots, leaves, pollen, seeds, tumor tissue and various forms of cells in culture (e.g., single cells, protoplasts, embryos, and callus tissue). The plant tissue may be in a plant or in a plant organ, tissue, or cell culture. Grain is intended to mean the mature seed produced by commercial growers for purposes other than growing or reproducing the species. Progeny, variants and mutants of the regenerated plants are also included within the scope of the disclosure, provided these progeny, variants and mutants comprise the introduced polynucleotides.

The present disclosure may be used for transformation of any plant species, including, but not limited to, monocots and dicots. Monocots include, but are not limited to, barley, maize (corn), millet (e.g., pearl millet (Pennisetum glaucum), proso millet (Panicum miliaceum), foxtail millet (Setaria italica), finger millet (Eleusine coracana), teff (Eragrostis tef), oats, rice, rye, Setaria sp., sorghum, triticale, or wheat, or leaf and stem crops, including, but not limited to, bamboo, marram grass, meadow-grass, reeds, ryegrass, sugarcane; lawn grasses, ornamental grasses, and other grasses such as switchgrass and turf grass. Alternatively, dicot plants used in the present disclosure, include, but are not limited to, kale, cauliflower, broccoli, mustard plant, cabbage, pea, clover, alfalfa, broad bean, tomato, peanut, cassava, soybean, canola, alfalfa, sunflower, safflower, tobacco, Arabidopsis, or cotton.

Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Higher plants, e.g., classes of Angiospermae and Gymnospermae may be used the present disclosure. Plants of suitable species useful in the present disclosure may come from the family Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, and Vitaceae. Plants from members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea may be used in the methods of the disclosure.

Plants important or interesting for agriculture, horticulture, biomass production (for production of liquid fuel molecules and other chemicals), and/or forestry may be used in the methods of the disclosure. Non-limiting examples include, for instance, Panicum virgatum (switchgrass), Miscanthus giganteus (miscanthus), Saccharum spp. (sugarcane, energycane), Populus balsamifera (poplar), cotton (Gossypium barbadense, Gossypium hirsutum), Helianthus annuus (sunflower), Medicago sativa (alfalfa), Beta vulgaris (sugarbeet), sorghum (Sorghum bicolor, Sorghum vulgare), Erianthus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus, including E. grandis (and its hybrids, known as “urograndis”), E. globulus, E. camaldulensis, E. tereticornis, E. viminalis, E. nitens, E. saligna and E. urophylla), Triticosecale spp. (triticum—wheat X rye), teff (Eragrostis tef), Bamboo, Carthamus tinctorius (safflower), Jatropha curcas (jatropha), Ricinus communis (castor), Elaeis guineensis (palm), Linum usitatissimum (flax), Manihot esculenta (cassava), Lycopersicon esculentum (tomato), Lactuca sativa (lettuce), Phaseolus vulgaris (green beans), Phaseolus limensis (lima beans), Lathyrus spp. (peas), Musa paradisiaca (banana), Solanum tuberosum (potato), Brassica spp. (B. napus (canola), B. rapa, B. juncea), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Arachis hypogaea (peanuts), Ipomoea batatus (sweet potato), Cocos nucifera (coconut), Citrus spp. (citrus trees), Persea americana (avocado), fig (Ficus casica), guava (Psidium guajava), mango (Mangifera indica), olive (Olea europaea), Carica papaya (papaya), Anacardium occidentale (cashew), Macadamia integrifolia (macadamia tree), Prunus amygdalus (almond), Allium cepa (onion), Cucumis melo (musk melon), Cucumis sativus (cucumber), Cucumis cantalupensis (cantaloupe), Cucurbita maxima (squash), Cucurbita moschata (squash), Spinacea oleracea (spinach), Citrullus lanatus (watermelon), Abelmoschus esculentus (okra), Solanum melongena (eggplant), Cyamopsis tetragonoloba (guar bean), Ceratonia siliqua (locust bean), Trigonella foenum-graecum (fenugreek), Vigna radiata (mung bean), Vigna unguiculata (cowpea), Vicia faba (fava bean), Cicer arietinum (chickpea), Lens culinaris (lentil), Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica., Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana (achiote), Alstroemeria spp., Rosa spp. (rose), Rhododendron spp. (azalea), Macrophylla hydrangea (hydrangea), Hibiscus rosasanensis (hibiscus), Tulipa spp. (tulips), Narcissus spp. (daffodils), Petunia hybrida (petunias), Dianthus caryophyllus (carnation), Euphorbia pulcherrima (poinsettia), chrysanthemum, Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers.

Conifers may be used in the present disclosure and include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Eastern or Canadian hemlock (Tsuga canadensis); Western hemlock (Tsuga heterophylla); Mountain hemlock (Tsuga mertensiana); Tamarack or Larch (Larix occidentalis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turf grasses may be used in the present disclosure and include, but are not limited to: annual bluegrass (Poa annua); annual ryegrass (Lolium multiflorum); Canada bluegrass (Poa compressa); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerata); perennial ryegrass (Lolium perenne); red fescue (Festuca rubra); redtop (Agrostis alba); rough bluegrass (Poa trivialis); sheep fescue (Festuca ovina); smooth bromegrass (Bromus inermis); timothy (Phleum pratense); velvet bentgrass (Agrostis canina); weeping alkaligrass (Puccinellia distans); western wheatgrass (Agropyron smithii); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

In specific aspects, plants transformed by the methods of the present disclosure are crop plants (for example, corn, alfalfa, sunflower, Brassica, soybean, cotton, safflower, peanut, rice. sorghum, wheat, millet, tobacco, etc.). Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include, but are not limited to, beans and peas. Beans include, but are not limited to, guar, locust bean, fenugreek, soybean, garden beans, cowpea, mungbean, lima bean, fava bean, lentils, and chickpea.

The present disclosure also includes plants obtained by any of the disclosed methods herein. The present disclosure also includes seeds from a plant obtained by any of the disclosed methods herein. A transgenic plant is defined as a mature, fertile plant that contains a transgene. In the disclosed methods, various plant-derived explants can be used, including immature embryos, 1-5 mm zygotic embryos, 3-5 mm embryos, and embryos derived from mature ear-derived seed, leaf bases, leaves from mature plants, leaf tips, immature influorescences, tassel, immature ear, and silks. In an aspect, the explants used in the disclosed methods can be derived from mature ear-derived seed, leaf bases, leaves from mature plants, leaf tips, immature influorescences, tassel, immature ear, and silks. The explant used in the disclosed methods can be derived from any of the plants described herein.

The disclosure encompasses isolated or substantially purified nucleic acid compositions. An “isolated” or “purified” nucleic acid molecule or protein or a biologically active portion thereof is substantially free of other cellular material or components that normally accompany or interact with the nucleic acid molecule or protein as found in its naturally occurring environment or is substantially free of culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various aspects, an isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When a protein useful in the methods of the disclosure or biologically active portion thereof is recombinantly produced, optimally culture medium represents less than about 30%, 20%, 10%, 5%, or 1% (by dry weight) of chemical precursors or non-protein-of-interest chemicals. Sequences useful in the methods of the disclosure may be isolated from the 5′ untranslated region flanking their respective transcription initiation sites. The present disclosure encompasses isolated or substantially purified nucleic acid or protein compositions useful in the methods of the disclosure.

As used herein, the term “fragment” refers to a portion of the nucleic acid sequence. Fragments of sequences useful in the methods of the disclosure retain the biological activity of the nucleic acid sequence. Alternatively, fragments of a nucleotide sequence that are useful as hybridization probes may not necessarily retain biological activity. Fragments of a nucleotide sequence disclosed herein may range from at least about 20, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, 4025, 4050, 4075, 4100, 4125, 4150, 4175, 4200, 4225, 4250, 4275, 4300, 4325, 4350, 4375, 4400, 4425, 4450, 4475, 4500, 4525, 4550, 4575, 4600, 4625, 4650, 4675, 4700, 4725, 4750, 4775, 4800, 4825, 4850, 4875, 4900, 4925, 4950, 4975, 5000, 5025, 5050, 5075, 5100, 5125, 5150, 5175, 5200, 5225, 5250, 5275, 5300, 5325, 5350, 5375, 5400, 5425, 5450, 5475, 5500, 5525, 5550, 5575, 5600, 5625, 5650, 5675, 5700, 5725, 5750, 5775, 5800, 5825, 5850, 5875, 5900, 5925, 5950, 5975, 6000, 6025, 6050, 6075, 6100, 6125, 6150, 6175, 6200, or 6225 nucleotides, and up to the full length of the subject sequence. A biologically active portion of a nucleotide sequence can be prepared by isolating a portion of the sequence, and assessing the activity of the portion.

Fragments and variants of nucleotide sequences and the proteins encoded thereby useful in the methods of the present disclosure are also encompassed. As used herein, the term “fragment” refers to a portion of a nucleotide sequence and hence the protein encoded thereby or a portion of an amino acid sequence. Fragments of a nucleotide sequence may encode protein fragments that retain the biological activity of the native protein. Alternatively, fragments of a nucleotide sequence useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins useful in the methods of the disclosure.

As used herein, the term “variants” is means sequences having substantial similarity with a sequence disclosed herein. A variant comprises a deletion and/or addition of one or more nucleotides or peptides at one or more internal sites within the native polynucleotide or polypeptide and/or a substitution of one or more nucleotides or peptides at one or more sites in the native polynucleotide or polypeptide. As used herein, a “native” nucleotide or peptide sequence comprises a naturally occurring nucleotide or peptide sequence, respectively. For nucleotide sequences, naturally occurring variants can be identified with the use of well-known molecular biology techniques, such as, for example, with polymerase chain reaction (PCR) and hybridization techniques as outlined herein. A biologically active variant of a protein useful in the methods of the disclosure may differ from that native protein by as few as 1-15 amino acid residues, as few as 1-10, such as 6-10, as few as 5, as few as 4, 3, 2, or even 1 amino acid residue.

Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biological activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook,” herein incorporated by reference in its entirety. Alternatively, levels of a reporter gene such as green fluorescent protein (GFP) or yellow fluorescent protein (YFP) or the like produced under the control of a promoter operably linked to a nucleotide fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90. Variant nucleotide sequences also encompass sequences derived from a mutagenic and recombinogenic procedure such as DNA shuffling. With such a procedure, one or more different nucleotide sequences can be manipulated to create a new nucleotide sequence. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The nucleotide sequences of the disclosure can be used to isolate corresponding sequences from other organisms, particularly other plants, more particularly other monocots or dicots. In this manner, methods such as PCR, hybridization and the like can be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as 32P or any other detectable marker. Thus, for example, probes for hybridization can be made by labeling synthetic oligonucleotides based on the sequences of the disclosure. Methods for preparation of probes for hybridization and for construction of genomic libraries are generally known in the art and are disclosed in Sambrook, supra.

For example, an entire sequence disclosed herein, or one or more portions thereof, may be used as a probe capable of specifically hybridizing to corresponding sequences and messenger RNAs. To achieve specific hybridization under a variety of conditions, such probes include sequences that are unique among sequences and are generally at least about 10 nucleotides in length or at least about 20 nucleotides in length. Such probes may be used to amplify corresponding sequences from a chosen plant by PCR. This technique may be used to isolate additional coding sequences from a desired organism or as a diagnostic assay to determine the presence of coding sequences in an organism. Hybridization techniques include hybridization screening of plated DNA libraries (either plaques or colonies, see, for example, Sambrook, supra).

Hybridization of such sequences may be carried out under stringent conditions. The terms “stringent conditions” or “stringent hybridization conditions” are intended to mean conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C. and a wash in 1 times to 2 times SSC (20 times SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C. and a wash in 0.5 times to 1 times SSC at 55 to 60° C. Exemplary high stringency conditions include hybridization in 50% formamide, 1 M NaCl, 1% SDS at 37° C., and a final wash in 0.1 times SSC at 60 to 65° C. for a duration of at least 30 minutes. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the thermal melting point (Tm) can be approximated from the equation of Meinkoth and Wahl, (1984) Anal. Biochem 138:267 284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching, thus, Tm, hybridization, and/or wash conditions can be adjusted to hybridize to sequences of the desired identity. For example, if sequences with 90% identity are sought, the Tm can be decreased 10° C. Generally, stringent conditions are selected to be about 5° C. lower than the Tm for the specific sequence and its complement at a defined ionic strength and pH. However, severely stringent conditions can utilize a hybridization and/or wash at 1, 2, 3 or 4° C. lower than the Tm; moderately stringent conditions can utilize a hybridization and/or wash at 6, 7, 8, 9 or 10° C. lower than the Tm; low stringency conditions can utilize a hybridization and/or wash at 11, 12, 13, 14, 15 or 20° C. lower than the Tm. Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, New York); and Ausubel, et al., eds. (1995) Current Protocols in Molecular Biology, Chapter 2 (Greene Publishing and Wiley-Interscience, New York), herein incorporated by reference in their entirety. See also, Sambrook supra. Thus, isolated sequences that have activity and which hybridize under stringent conditions to the sequences disclosed herein or to fragments thereof, are encompassed by the present disclosure. In general, sequences that have activity and hybridize to the sequences disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”. As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” refers to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244; Higgins, et al., (1989) CABIOS 5:151-153; Corpet, et al., (1988) Nucleic Acids Res. 16:10881-90; Huang, et al., (1992) CABIOS 8:155-65; and Pearson, et al., (1994) Meth. Mol. Biol. 24:307-331, herein incorporated by reference in their entirety. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403, herein incorporated by reference in its entirety, are based on the algorithm of Karlin and Altschul, (1990) supra. BLAST nucleotide searches can be performed with the BLASTN program, score=100, word length=12, to obtain nucleotide sequences homologous to a nucleotide sequence encoding a protein of the disclosure. BLAST protein searches can be performed with the BLASTX program, score=50, word length=3, to obtain amino acid sequences homologous to a protein or polypeptide of the disclosure. To obtain gapped alignments for comparison purposes, Gapped BLAST (in BLAST 2.0) can be utilized as described in Altschul, et al., (1997) Nucleic Acids Res. 25:3389, herein incorporated by reference in its entirety. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, the web site for the National Center for Biotechnology Information on the World Wide Web at ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. As used herein, “equivalent program” is any sequence comparison program that, for any two sequences in question, generates an alignment having identical nucleotide or amino acid residue matches and an identical percent sequence identity when compared to the corresponding alignment generated by GAP Version 10.

The GAP program uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the Quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see, Henikoff and Henikoff, (1989) Proc. Natl. Acad. Sci. USA 89:10915, herein incorporated by reference in its entirety).

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tm for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

“Variants” is intended to mean substantially similar sequences. For polynucleotides, conservative variants include those sequences that, because of the degeneracy of the genetic code, encode the amino acid sequence of one of the morphogenic genes and/or genes/polynucleotides of interest disclosed herein. Variant polynucleotides also include synthetically derived polynucleotides, such as those generated, for example, by using site-directed mutagenesis but which still encode a protein of a morphogenic gene and/or gene/polynucleotide of interest disclosed herein. Generally, variants of a particular morphogenic gene and/or gene/polynucleotide of interest disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular morphogenic gene and/or gene/polynucleotide of interest as determined by sequence alignment programs and parameters described elsewhere herein.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present disclosure are biologically active, that is they continue to possess the desired biological activity of the native protein, that is, the polypeptide has morphogenic gene and/or gene/polynucleotide of interest activity. Such variants may result from, for example, genetic polymorphism or from human manipulation. Biologically active variants of a native morphogenic gene and/or gene/polynucleotide of interest protein disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue.

The sequences and genes disclosed herein, as well as variants and fragments thereof, are useful for the genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), plastid transformation (see, for example Zora Svab, Peter Hajdukiewicz, and Pal Maliga (1990) Stable transformation of plastids in higher plants, Proc. Natl. Acad. Sci. 87:8526-8530) and in U.S. Pat. No. 5,877,402, incorporated herein by reference in their entireties, and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin) (maize); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255; Christou and Ford (1995) Annals of Botany 75:407-413 (rice); Osjoda et al. (1996) Nature Biotechnology 14:745-750 (maize via Agrobacterium tumefaciens); and US Patent Application Publication Number 2017/0121722 (rapid plant transformation) all of which are herein incorporated by reference in their entireties.

The methods provided herein rely upon the use of bacteria-mediated and/or biolistic-mediated gene transfer to produce regenerable plant cells having an incorporated nucleotide sequence of interest. Bacterial strains useful in the methods of the disclosure include, but are not limited to, a disarmed Agrobacteria, an Ochrobactrum bacteria or a Rhizobiaceae bacteria.

Disarmed Agrobacteria useful in the present methods include, but are not limited to, AGL-1, EHA105, GV3101, LBA4404, and LBA4404 THY-.

Ochrobactrum bacterial strains useful in the present methods include, but are not limited to, those listed in Table 2 (see also U.S. Patent Appln. No. 20180216123 incorporated herein by reference in its entirety).

TABLE 2

Ochrobactrum haywardense H1 NRRL Deposit B-67078
Ochrobactrum cytisi
Ochrobactrum daejeonense
Ochrobactrum oryzae
Ochrobactrum tritici LBNL124-A-10
HTG3-C-07
Ochrobactrum pecoris
Ochrobactrum ciceri
Ochrobactrum gallinifaecis
Ochrobactrum grignonense
Ochrobactrum guangzhouense
Ochrobactrum haematophilum
Ochrobactrum intermedium
Ochrobactrum lupini
Ochrobactrum pituitosum
Ochrobactrum pseudintermedium
Ochrobactrum pseudogrignonense
Ochrobactrum rhizosphaerae
Ochrobactrum thiophenivorans
Ochrobactrum tritici

Rhizobiaceae bacterial strains useful in the present methods include, but are not limited to, those listed in Table 3 (see also U.S. Pat. No. 9,365,859 incorporated herein by reference in its entirety).

TABLE 3

Rhizobium lusitanum
Rhizobium rhizogenes
Agrobacterium rubi
Rhizobium multihospitium
Rhizobium tropici
Rhizobium miluonense
Rhizobium leguminosarum
Rhizobium leguminosarum bv. trifolii
Rhizobium leguminosarum bv. phaseoli
Rhizobium leguminosarum. bv. viciae
Rhizobium leguminosarum Madison
Rhizobium leguminosarum USDA2370
Rhizobium leguminosarum USDA2408
Rhizobium leguminosarum USDA2668
Rhizobium leguminosarum 2370G
Rhizobium leguminosarum 2370LBA
Rhizobium leguminosarum 2048G
Rhizobium leguminosarum 2048LBA
Rhizobium leguminosarum bv. phaseoli 2668G
Rhizobium leguminosarum bv. phaseoli 2668LBA
Rhizobium leguminosarum RL542C
Rhizobium etli USDA 9032
Rhizobium etli bv. phaseoli
Rhizobium endophyticum
Rhizobium tibeticum
Rhizobium etli
Rhizobium pisi
Rhizobium phaseoli
Rhizobium fabae
Rhizobium hainanense
Arthrobacter viscosus
Rhizobium alamii
Rhizobium mesosinicum
Rhizobium sullae
Rhizobium indigoferae
Rhizobium gallicum
Rhizobium yanglingense
Rhizobium mongolense
Rhizobium oryzae
Rhizobium loessense
Rhizobium tubonense
Rhizobium cellulosilyticum
Rhizobium soli
Neorhizobium galegae
Neorhizobium vignae
Neorhizobium huautlense
Neorhizobium alkalisoli
Aureimonas altamirensis
Aureimonas frigidaquae
Aureimonas ureilytica. Aurantimoncis coralicida
Fulvimarina pelagi
Martelella mediterranea
Allorhizobium undicola
Allorhizobium vitis
Allorhizobium borbor
Beijerinckia fluminensis
Agrobacterium larrymoorei
Agrobacterium radiobacter
Rhizobium selenitireducens corrig. Rhizobium rosettiformans
Rhizobium daejeonense
Rhizobium aggregatum
Pararhizobium capsulatum
Pararhizobium giardinii
Ensifer mexicanus
Ensifer terangae
Ensifer saheli
Ensifer kostiensis
Ensifer kummerowiae
Ensifer fredii
Sinorhizobium cimericanum
Ensifer arboris
Ensifer garamanticus
Ensifer meliloti
Ensifer numidicus
Ensifer adhaerens
Sinorhizobium sp.
Sinorhizobium meliloti SD630
Sinorhizobium meliloti USDA1002
Sinorhizobium fredii USDA205
Sinorhizobium fredii SF542G
Sinorhizobium fredii SF4404
Sinorhizobium fredii SM542C.

The polynucleotide of the disclosure may be introduced into plants by contacting plants with a virus or viral nucleic acids. Generally, such methods involve incorporating a nucleotide construct of the disclosure within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference in their entirety.

The methods of the disclosure involve introducing a polypeptide or polynucleotide into a plant. As used herein, “introducing” means presenting to the plant the polynucleotide or polypeptide in such a manner that the sequence gains access to the interior of a cell of the plant. The methods of the disclosure do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

A “stable transformation” is a transformation in which the nucleotide construct introduced into a plant integrates into the genome of the plant and is capable of being inherited by the progeny thereof. “Transient transformation” means that a polynucleotide is introduced into the plant and does not integrate into the genome of the plant or a polypeptide is introduced into a plant.

Reporter genes or selectable marker genes may also be included in the expression cassettes and used in the methods of the disclosure. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

A selectable marker comprises a DNA segment that allows one to identify or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include, but are not limited to, DNA segments that comprise restriction enzyme sites; DNA segments that encode products which provide resistance against otherwise toxic compounds (e.g., antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Selectable markers that confer resistance to herbicidal compounds include genes encoding resistance and/or tolerance to herbicidal compounds, such as glyphosate, sulfonylureas, glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillen and Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

Certain seletable markers useful in the present method include, but are not limited to, the maize HRA gene (Lee et al., 1988, EMBO J 7:1241-1248) which confers resistance to sulfonylureas and imidazolinones, the GAT gene which confers resistance to glyphosate (Castle et al., 2004, Science 304:1151-1154), genes that confer resistance to spectinomycin such as the aadA gene (Svab et al., 1990, Plant Mol Biol. 14:197-205) and the bar gene that confers resistance to glufosinate ammonium (White et al., 1990, Nucl. Acids Res. 25:1062), and PAT (or moPAT for corn, see Rasco-Gaunt et al., 2003, Plant Cell Rep.21:569-76) and the PMI gene that permits growth on mannose-containing medium (Negrotto et al., 2000, Plant Cell Rep. 22:684-690) are very useful for rapid selection during the brief elapsed time encompassed by somatic embryogenesis and embry maturation of the method. However, depending on the selectable marker used and the crop, inbred or variety being transformed, the percentage of wild-type escapes can vary. In maize and sorghum, the HRA gene is efficacious in reducing the frequency of wild-type escapes.

Other genes that could have utility in the recovery of transgenic events would include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414), various fluorescent proteins with a spectrum of alternative emission optima spanning Far-Red, Red, Orange, Yellow, Green Cyan and Blue (Shaner et al., 2005, Nature Methods 2:905-909) and the maize genes encoding for anthocyanin production (Ludwig, et al., (1990) Science 247:449), herein incorporated by reference in their entireties.

The above list of selectable markers is not meant to be limiting. Any selectable marker can be used in the methods of the disclosure.

In an aspect, the methods of the disclosure provide transformation methods that allow positive growth selection. One skilled in the art can appreciate that conventional plant transformation methods have relied predominantly on negative selection schemes as described above, in which an antibiotic or herbicide (a negative selective agent) is used to inhibit or kill non-transformed cells or tissues, and the transgenic cells or tissues continue to grow due to expression of a resistance gene. In contrast, the methods of the present disclosure can be used with no application of a negative selective agent. Thus, although wild-type cells can grow unhindered, by comparison cells impacted by the controlled expression of a morphogenic gene can be readily identified due to their accelerated growth rate relative to the surrounding wild-type tissue. In addition to simply observing faster growth, the methods of the disclosure provide transgenic cells that exhibit more rapid morphogenesis relative to non-transformed cells. Accordingly, such differential growth and morphogenic development can be used to easily distinguish transgenic plant structures from the surrounding non-transformed tissue, a process which is termed herein as “positive growth selection.”

The present disclosure provides methods for producing transgenic plants with increased efficiency and speed and providing significantly higher transformation frequencies and significantly more quality events (events containing one copy of a trait gene cassette with no vector (plasmid) backbone) in multiple inbred lines using a variety of starting tissue types, including transformed inbreds representing a range of genetic diversities and having significant commercial utility. The disclosed methods can further comprise polynucleotides that provide for improved traits and characteristics.

As used herein, “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference) could be used. Other agronomically important genes encode latex, Floury 2, growth factors, seed storage factors, and transcription factors.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

An “enhanced trait” as used in describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, improved seed quality, enhanced nitrogen use efficiency, early plant growth and development, late plant growth and development, enhanced seed protein, and enhanced seed oil production.

Any polynucleotide of interest can be used in the methods of the disclosure. Various changes in phenotype, imparted by a gene of interest, include those for modifying the fatty acid composition in a plant, altering the amino acid content, starch content, or carbohydrate content of a plant, altering a plant's pathogen defense mechanism, altering kernel size, altering sucrose loading, and the like. The gene of interest may also be involved in regulating the influx of nutrients, and in regulating expression of phytate genes particularly to lower phytate levels in the seed. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as the understanding of agronomic traits and characteristics such as yield and heterosis increase, the choice of genes for transformation will change accordingly. General categories of nucleotide sequences or genes of interest usefil in the methods of the disclosure include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, environmental stress resistance (altered tolerance to cold, salt, drought, etc.), grain characteristics, and commercial products.

Heterologous coding sequences, heterologous polynucleotides, and polynucleotides of interest expressed by a promoter sequence transformed by the methods disclosed herein may be used for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results can be achieved by the expression of a heterologous nucleotide sequence of interest comprising an appropriate gene product. In specific aspects, the heterologous nucleotide sequence of interest is an endogenous plant sequence whose expression level is increased in the plant or plant part. Results can be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant. Still other categories of transgenes include genes for inducing expression of exogenous products such as enzymes, cofactors, and hormones from plants and other eukaryotes as well as prokaryotic organisms.

It is recognized that any gene of interest, polynucleotide of interest, or multiple genes/polynucleotides of interest can be operably linked to a promoter or promoters and expressed in a plant transformed by the methods disclosed herein, for example insect resistance traits which can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like).

A promoter can be operably linked to agronomically important traits for expression in plants transformed by the methods disclosed herein that affect quality of grain, such as levels (increasing content of oleic acid) and types of oils, saturated and unsaturated, quality and quantity of essential amino acids, increasing levels of lysine and sulfur, levels of cellulose, and starch and protein content. A promoter can be operably linked to genes providing hordothionin protein modifications for expression in plants transformed by the methods disclosed herein which are described in U.S. Pat. Nos. 5,990,389; 5,885,801; 5,885,802 and 5,703,049; herein incorporated by reference in their entirety. Another example of a gene to which a promoter can be operably linked to for expression in plants transformed by the methods disclosed herein is a lysine and/or sulfur rich seed protein encoded by the soybean 2S albumin described in U.S. Pat. No. 5,850,016, and the chymotrypsin inhibitor from barley, Williamson, et al., (1987) Eur. J. Biochem 165:99-106, the disclosures of which are herein incorporated by reference in their entirety.

A promoter can be operably linked to insect resistance genes that encode resistance to pests that have yield drag such as rootworm, cutworm, European corn borer and the like for expression in plants transformed by the methods disclosed herein. Such genes include, for example, Bacillus thuringiensis toxic protein genes, U.S. Pat. Nos. 5,366,892; 5,747,450; 5,736,514; 5,723,756; 5,593,881 and Geiser, et al., (1986) Gene 48:109, the disclosures of which are herein incorporated by reference in their entirety. Genes encoding disease resistance traits that can be operably linked to a promoter for expression in plants transformed by the methods disclosed herein include, for example, detoxification genes, such as those which detoxify fumonisin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089), herein incorporated by reference in their entirety.

Herbicide resistance traits that can be operably linked to a promoter for expression in plants transformed by the methods disclosed herein include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), genes coding for resistance to glyphosate (e.g., the EPSPS gene and the GAT gene; see, for example, US Patent Application Publication Number 2004/0082770, WO 03/092360 and WO 05/012515, herein incorporated by reference in their entirety) or other such genes known in the art. The bar gene encodes resistance to the herbicide basta, the nptll gene encodes resistance to the antibiotics kanamycin and geneticin and the ALS-gene mutants encode resistance to the herbicide chlorsulfuron any and all of which can be operably linked to a promoter for expression in plants transformed by the methods disclosed herein.

Glyphosate resistance is imparted by mutant 5-enolpyruvl-3-phosphikimate synthase (EPSPS) and aroA genes which can be operably linked to a promoter for expression in plants transformed by the methods disclosed herein. See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes which can be operably linked to a promoter for expression in plants transformed by the methods disclosed herein. See also, U.S. Pat. Nos. 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and international publications WO 97/04103; WO 97/04114; WO 00/66746; WO 01/66704; WO 00/66747 and WO 00/66748, which are incorporated herein by reference in their entirety. Glyphosate resistance is also imparted to plants that express a gene which can be operably linked to a promoter for expression in plants transformed by the methods disclosed herein that encodes a glyphosate oxido-reductase enzyme as described more fully in U.S. Pat. Nos. 5,776,760 and 5,463,175, which are incorporated herein by reference in their entirety. Glyphosate resistance can also be imparted to plants by the over expression of genes which can be operably linked to a promoter for expression in plants transformed by the methods disclosed herein encoding glyphosate N-acetyltransferase. See, for example, US Patent Application Publication Number 2004/0082770, WO 03/092360 and WO 05/012515, herein incorporated by reference in their entirety.

Sterility genes operably linked to a promoter for expression in plants transformed by the methods disclosed herein can also be encoded in a DNA construct and provide an alternative to physical detasseling. Examples of genes used in such ways include male tissue-preferred genes and genes with male sterility phenotypes such as QM, described in U.S. Pat. No. 5,583,210, herein incorporated by reference in its entirety. Other genes which can be operably linked to a promoter for expression in plants transformed by the methods disclosed herein include kinases and those encoding compounds toxic to either male or female gametophytic development.

Commercial traits can also be encoded by a gene or genes operably linked to a promoter for expression in plants transformed by the methods disclosed herein that could increase for example, starch for ethanol production, or provide expression of proteins. Another important commercial use of transformed plants is the production of polymers and bioplastics such as described in U.S. Pat. No. 5,602,321, herein incorporated by reference in its entirety. Genes such as β-Ketothiolase, PHBase (polyhydroxybutyrate synthase), and acetoacetyl-CoA reductase, which facilitate expression of polyhydroxyalkanoates (PHAs) can be operably linked to a promoter for expression in plants transformed by the methods disclosed herein (see, Schubert, et al., (1988) J. Bacteriol. 170:5837-5847, herein incorporated by reference in its entirety).

Examples of other applicable genes and their associated phenotype which can be operably linked to a promoter for expression in plants transformed by the methods disclosed herein include genes that encode viral coat proteins and/or RNAs, or other viral or plant genes that confer viral resistance; genes that confer fungal resistance; genes that promote yield improvement; and genes that provide for resistance to stress, such as cold, dehydration resulting from drought, heat and salinity, toxic metal or trace elements or the like.

By way of illustration, without intending to be limiting, the following is a list of other examples of the types of genes which can be operably linked to a promoter for expression in plants transformed by the methods disclosed herein.

1. Transgenes that Confer Resistance to Insects or Disease and that Encode:
(A) Plant disease resistance genes. Plant defenses are often activated by specific interaction between the product of a disease resistance gene (R) in the plant and the product of a corresponding avirulence (Avr) gene in the pathogen. A plant variety can be transformed with a cloned resistance gene to engineer plants that are resistant to specific pathogen strains. See, for example Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae); McDowell and Woffenden, (2003) Trends Biotechnol. 21(4):178-83 and Toyoda, et al., (2002) Transgenic Res. 11(6):567-82, herein incorporated by reference in their entirety. A plant resistant to a disease is one that is more resistant to a pathogen as compared to the wild type plant.
(B) A Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Numbers 40098, 67136, 31995 and 31998. Other examples of Bacillus thuringiensis transgenes being genetically engineered are given in the following patents and patent applications and hereby are incorporated by reference for this purpose: U.S. Pat. Nos. 5,188,960; 5,689,052; 5,880,275; WO 91/14778; WO 99/31248; WO 01/12731; WO 99/24581; WO 97/40162 and U.S. application Ser. Nos. 10/032,717; 10/414,637 and 10/606,320, herein incorporated by reference in their entirety.
(C) An insect-specific hormone or pheromone such as an ecdysteroid and juvenile hormone, a variant thereof, a mimetic based thereon, or an antagonist or agonist thereof. See, for example, the disclosure by Hammock, et al., (1990) Nature 344:458, of baculovirus expression of cloned juvenile hormone esterase, an inactivator of juvenile hormone, herein incorporated by reference in its entirety.
(D) An insect-specific peptide which, upon expression, disrupts the physiology of the affected pest. For example, see the disclosures of Regan, (1994) J. Biol. Chem. 269:9 (expression cloning yields DNA coding for insect diuretic hormone receptor); Pratt, et al., (1989) Biochem. Biophys. Res. Comm.163:1243 (an allostatin is identified in Diploptera puntata); Chattopadhyay, et al., (2004) Critical Reviews in Microbiology 30(1):33-54; Zjawiony, (2004) J Nat Prod 67(2):300-310; Carlini and Grossi-de-Sa, (2002) Toxicon 40(11):1515-1539; Ussuf, et al., (2001) Curr Sci. 80(7):847-853 and Vasconcelos and Oliveira, (2004) Toxicon 44(4):385-403, herein incorporated by reference in their entirety. See also, U.S. Pat. No. 5,266,317 to Tomalski, et al., who disclose genes encoding insect-specific toxins, herein incorporated by reference in its entirety.
(E) An enzyme responsible for a hyperaccumulation of a monterpene, a sesquiterpene, a steroid, hydroxamic acid, a phenylpropanoid derivative or another non-protein molecule with insecticidal activity.
(F) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application Number WO 93/02197 in the name of Scott, et al., which discloses the nucleotide sequence of a callase gene, herein incorporated by reference in its entirety. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase, and Kawalleck, et al., (1993) Plant Molec. Biol. 21:673, who provide the nucleotide sequence of the parsley ubi4-2 polyubiquitin gene, U.S. patent application Ser. Nos. 10/389,432, 10/692,367 and U.S. Pat. No. 6,563,020, herein incorporated by reference in their entirety.
(G) A molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone, herein incorporated by reference in their entirety.
(H) A hydrophobic moment peptide. See, PCT Application Number WO 95/16776 and U.S. Pat. No. 5,580,852 (disclosure of peptide derivatives of Tachyplesin which inhibit fungal plant pathogens) and PCT Application Number WO 95/18855 and U.S. Pat. No. 5,607,914) (teaches synthetic antimicrobial peptides that confer disease resistance), herein incorporated by reference in their entirety.
(I) A membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum, herein incorporated by reference in its entirety.
(J) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451, herein incorporated by reference in its entirety. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.
(K) An insect-specific antibody or an immunotoxin derived therefrom. Thus, an antibody targeted to a critical metabolic function in the insect gut would inactivate an affected enzyme, killing the insect. Cf Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments), herein incorporated by reference in its entirety.
(L) A virus-specific antibody. See, for example, Tavladoraki, et al., (1993) Nature 366:469, who show that transgenic plants expressing recombinant antibody genes are protected from virus attack, herein incorporated by reference in its entirety.
(M) A developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436, herein incorporated by reference in its entirety. The cloning and characterization of a gene which encodes a bean endopolygalacturonase-inhibiting protein is described by Toubart, et al., (1992) Plant J. 2:367, herein incorporated by reference in its entirety.
(N) A developmental-arrestive protein produced in nature by a plant. For example, Logemann, et al., (1992) Bio/Technology 10:305, herein incorporated by reference in its entirety, have shown that transgenic plants expressing the barley ribosome-inactivating gene have an increased resistance to fungal disease.
(O) Genes involved in the Systemic Acquired Resistance (SAR) Response and/or the pathogenesis related genes. Briggs, (1995) Current Biology 5(2):128-131, Pieterse and Van Loon, (2004) Curr. Opin. Plant Bio. 7(4):456-64 and Somssich, (2003) Cell 113(7):815-6, herein incorporated by reference in their entirety.
(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. No. 09/950,933, herein incorporated by reference in their entirety.
(Q) Detoxification genes, such as for fumonisin, beauvericin, moniliformin and zearalenone and their structurally related derivatives. For example, see, U.S. Pat. No. 5,792,931, herein incorporated by reference in its entirety.
(R) Cystatin and cysteine proteinase inhibitors. See, U.S. application Ser. No. 10/947,979, herein incorporated by reference in its entirety.
(S) Defensin genes. See, WO03/000863 and U.S. application Ser. No. 10/178,213, herein incorporated by reference in their entirety.
(T) Genes conferring resistance to nematodes. See, WO 03/033651 and Urwin, et. al., (1998) Planta 204:472-479, Williamson (1999) Curr Opin Plant Bio. 2(4):327-31, herein incorporated by reference in their entirety.
(U) Genes such as rcg1 conferring resistance to Anthracnose stalk rot, which is caused by the fungus Colletotrichum graminiola. See, Jung, et al., Generation-means analysis and quantitative trait locus mapping of Anthracnose Stalk Rot genes in Maize, Theor. Appl. Genet. (1994) 89:413-418, as well as, U.S. Provisional Patent Application No. 60/675,664, herein incorporated by reference in their entirety.

2. Transgenes that Confer Resistance to a Herbicide, for Example:
(A) A herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824 and international publication WO 96/33270, which are incorporated herein by reference in their entirety.
(B) Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes) and pyridinoxy or phenoxy proprionic acids and cycloshexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and international publications EP1173580; WO 01/66704; EP1173581 and EP1173582, which are incorporated herein by reference in their entirety. Glyphosate resistance is also imparted to plants that express a gene that encodes a glyphosate oxido-reductase enzyme as described more fully in U.S. Pat. Nos. 5,776,760 and 5,463,175, which are incorporated herein by reference in their entirety. In addition, glyphosate resistance can be imparted to plants by the over expression of genes encoding glyphosate N-acetyltransferase. See, for example, US Patent Application Publication Number 2004/0082770, WO 03/092360 and WO 05/012515, herein incorporated by reference in their entirety. A DNA molecule encoding a mutant aroA gene can be obtained under ATCC Accession Number 39256 and the nucleotide sequence of the mutant gene is disclosed in U.S. Pat. No. 4,769,061 to Comai, herein incorporated by reference in its entirety. EP Patent Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin, herein incorporated by reference in their entirety. The nucleotide sequence of a phosphinothricin-acetyl-transferase gene is provided in EP Patent Numbers 0 242 246 and 0 242 236 to Leemans, et al., De Greef, et al., (1989) Bio/Technology 7:61 which describe the production of transgenic plants that express chimeric bar genes coding for phosphinothricin acetyl transferase activity, herein incorporated by reference in their entirety. See also, U.S. Pat. Nos. 5,969,213; 5,489,520; 5,550,318; 5,874,265; 5,919,675; 5,561,236; 5,648,477; 5,646,024; 6,177,616 B1 and 5,879,903, herein incorporated by reference in their entirety. Exemplary genes conferring resistance to phenoxy proprionic acids and cycloshexones, such as sethoxydim and haloxyfop, are the Acc1-S1, Acc1-S2 and Acc1-S3 genes described by Marshall, et al., (1992) Theor. Appl. Genet. 83:435, herein incorporated by reference in its entirety.
(C) A herbicide that inhibits photosynthesis, such as a triazine (psbA and gs+ genes) and a benzonitrile (nitrilase gene). Przibilla, et al., (1991) Plant Cell 3:169, herein incorporated by reference in its entirety, describe the transformation of Chlamydomonas with plasmids encoding mutant psbA genes. Nucleotide sequences for nitrilase genes are disclosed in U.S. Pat. No. 4,810,648 to Stalker, herein incorporated by reference in its entirety, and DNA molecules containing these genes are available under ATCC Accession Numbers 53435, 67441 and 67442. Cloning and expression of DNA coding for a glutathione S-transferase is described by Hayes, et al., (1992) Biochem. J. 285:173, herein incorporated by reference in its entirety.
(D) Acetohydroxy acid synthase, which has been found to make plants that express this enzyme resistant to multiple types of herbicides, has been introduced into a variety of plants (see, e.g., Hattori, et al., (1995) Mol Gen Genet 246:419, herein incorporated by reference in its entirety). Other genes that confer resistance to herbicides include: a gene encoding a chimeric protein of rat cytochrome P4507A1 and yeast NADPH-cytochrome P450 oxidoreductase (Shiota, et al., (1994) Plant Physiol. 106(1):17-23), genes for glutathione reductase and superoxide dismutase (Aono, et al., (1995) Plant Cell Physiol 36:1687, and genes for various phosphotransferases (Datta, et al., (1992) Plant Mol Biol 20:619), herein incorporated by reference in their entirety.
(E) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373; and international publication number WO 01/12825, herein incorporated by reference in their entirety.

3. Transgenes that Confer or Contribute to an Altered Grain Characteristic, Such As:
(A) Altered fatty acids, for example, by
   (1) Down-regulation of stearoyl-ACP desaturase to increase stearic acid content of the plant. See, Knultzon, et al., (1992) Proc. Natl. Acad. Sci. USA 89:2624 and WO99/64579 (Genes for Desaturases to Alter Lipid Profiles in Corn), herein incorporated by reference in their entirety,
   (2) Elevating oleic acid via FAD-2 gene modification and/or decreasing linolenic acid via FAD-3 gene modification (see, U.S. Pat. Nos. 6,063,947; 6,323,392; 6,372,965 and WO 93/11245, herein incorporated by reference in their entirety),
   (3) Altering conjugated linolenic or linoleic acid content, such as in WO 01/12800, herein incorporated by reference in its entirety,
   (4) Altering LEC1, AGP, Dek1, Superal1, mi1ps, various 1pa genes such as lpa1, lpa3, hpt or hggt. For example, see, WO 02/42424, WO 98/22604, WO 03/011015, U.S. Pat. Nos. 6,423,886, 6,197,561, 6,825,397, US Patent Application Publication Numbers 2003/0079247, 2003/0204870, WO02/057439, WO03/011015 and Rivera-Madrid, et. al., (1995) Proc. Natl. Acad. Sci. 92:5620-5624, herein incorporated by reference in their entirety.
(B) Altered phosphorus content, for example, by the
   (1) Introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene, herein incorporated by reference in its entirety.
   (2) Up-regulation of a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy, et al., (1990) Maydica 35:383 and/or by altering inositol kinase activity as in WO 02/059324, US Patent Application Publication Number 2003/0009011, WO 03/027243, US Patent Application Publication Number 2003/0079247, WO 99/05298, U.S. Pat. Nos. 6,197,561, 6,291,224, 6,391,348, WO2002/059324, US Patent Application Publication Number 2003/0079247, WO98/45448, WO99/55882, WO01/04147, herein incorporated by reference in their entirety.
(C) Altered carbohydrates effected, for example, by altering a gene for an enzyme that affects the branching pattern of starch or a gene altering thioredoxin such as NTR and/or TRX (see, U.S. Pat. No. 6,531,648, which is incorporated by reference in its entirety) and/or a gamma zein knock out or mutant such as cs27 or TUSC27 or en27 (see, U.S. Pat. No. 6,858,778 and US Patent Application Publication Numbers 2005/0160488 and 2005/0204418; which are incorporated by reference in its entirety). See, Shiroza, et al., (1988) J. Bacteriol. 170:810 (nucleotide sequence of Streptococcus mutans fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)), herein incorporated by reference in their entirety. The fatty acid modification genes mentioned above may also be used to affect starch content and/or composition through the interrelationship of the starch and oil pathways.
(D) Altered antioxidant content or composition, such as alteration of tocopherol or tocotrienols. For example, see U.S. Pat. No. 6,787,683, US Patent Application Publication Number 2004/0034886 and WO 00/68393 involving the manipulation of antioxidant levels through alteration of a phytl prenyl transferase (ppt), WO 03/082899 through alteration of a homogentisate geranyl geranyl transferase (hggt), herein incorporated by reference in their entirety.
(E) Altered essential seed amino acids. For example, see U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US Patent Application Publication Number 2003/0163838, US Patent Application Publication Number 2003/0150014, US Patent Application Publication Number 2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US Patent Application Publication Number 2004/0025203 (UDPGdH), U.S. Pat. No. 6,194,638 (RGP), herein incorporated by reference in their entirety.

4. Genes that Create a Site for Site Specific DNA Integration

This includes the introduction of FRT sites that may be used in the FLP/FRT system and/or Lox sites that may be used in the Cre/Loxp system. For example, see Lyznik, et al., (2003) Plant Cell Rep 21:925-932 and WO 99/25821, which are hereby incorporated by reference in their entirety. Other systems that may be used include the Gin recombinase of phage Mu (Maeser, et al., 1991; Vicki Chandler, The Maize Handbook ch. 118 (Springer-Verlag 1994), the Pin recombinase of E. coli (Enomoto, et al., 1983), and the R/RS system of the pSR1 plasmid (Araki, et al., 1992), herein incorporated by reference in their entirety.

5. Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. For example, see, WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, and WO9938977 describing genes, including CBF genes and transcription factors effective in mitigating the negative effects of freezing, high salinity, and drought on plants, as well as conferring other positive effects on plant phenotype; US Patent Application Publication Number 2004/0148654 and WO01/36596 where abscisic acid is altered in plants resulting in improved plant phenotype such as increased yield and/or increased tolerance to abiotic stress; WO2000/006341, WO04/090143, U.S. patent application Ser. No. 10/817,483 and U.S. Pat. No. 6,992,237, where cytokinin expression is modified resulting in plants with increased stress tolerance, such as drought tolerance, and/or increased yield, herein incorporated by reference in their entirety. Also see WO0202776, WO2003052063, JP2002281975, U.S. Pat. No. 6,084,153, WO0164898, U.S. Pat. Nos. 6,177,275 and 6,107,547 (enhancement of nitrogen utilization and altered nitrogen responsiveness), herein incorporated by reference in their entirety. For ethylene alteration, see US Patent Application Publication Number 2004/0128719, US Patent Application Publication Number 2003/0166197 and WO200032761, herein incorporated by reference in their entirety. For plant transcription factors or transcriptional regulators of abiotic stress, see, e.g., US Patent Application Publication Number 2004/0098764 or US Patent Application Publication Number 2004/0078852, herein incorporated by reference in their entirety.

6. Other genes and transcription factors that affect plant growth and agronomic traits such as yield, flowering, plant growth and/or plant structure, can be introduced or introgressed into plants, see, e.g., WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. Nos. 6,794,560, 6,307,126 (GAI), WO99/09174 (D8 and Rht) and WO2004076638 and WO2004031349 (transcription factors), herein incorporated by reference in their entirety.

As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. “Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

A heterologous nucleotide sequence operably linked to a promoter and its related biologically active fragments or variants useful in the methods disclosed herein may be an antisense sequence for a targeted gene. The terminology “antisense DNA nucleotide sequence” is intended to mean a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having 70%, 80%, 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides or greater may be used. Thus, a promoter may be operably linked to antisense DNA sequences to reduce or inhibit expression of a native protein in the plant when transformed by the methods disclosed herein.

“RNAi” refers to a series of related techniques to reduce the expression of genes (see, for example, U.S. Pat. No. 6,506,559, herein incorporated by reference in its entirety). Older techniques referred to by other names are now thought to rely on the same mechanism, but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced.

As used herein, the terms “promoter” or “transcriptional initiation region” mean a regulatory region of DNA usually comprising a TATA box or a DNA sequence capable of directing RNA polymerase II to initiate RNA synthesis at the appropriate transcription initiation site for a particular coding sequence. A promoter may additionally comprise other recognition sequences generally positioned upstream or 5′ to the TATA box or the DNA sequence capable of directing RNA polymerase II to initiate RNA synthesis, referred to as upstream promoter elements, which influence the transcription initiation rate.

The transcriptional initiation region, the promoter, may be native or homologous or foreign or heterologous to the host, or could be the natural sequence or a synthetic sequence. By foreign is intended that the transcriptional initiation region is not found in the wild-type host into which the transcriptional initiation region is introduced. Either a native or heterologous promoter may be used with respect to the coding sequence of interest.

The transcriptional cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the potato proteinase inhibitor (PinII) gene or sequences from Ti-plasmid of A. tumefaciens, such as the nopaline synthase, octopine synthase and opaline synthase termination regions. See also, Guerineau et al., (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. 1989) Nucleic Acids Res. 17: 7891-7903; Joshi et al. (1987) Nucleic Acid Res. 15: 9627-9639.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′noncoding region) (Elroy-Stein, O., Fuerst, T. R., and Moss, B. (1989) PNAS USA, 86: 6126-6130); potyvirus leaders, for example, TEV leader (Tobacco Etch Virus) (Allison et al. (1986); MDMV leader (Maize Dwarf Mosaic Virus); Virology, 154: 9-20), and human immunoglobulin heavy-chain binding protein (BiP), (Macejak, D. G., and P. Sarnow (1991) Nature, 353: 90-94; untranslated leader from the coat protein MARNA of alfalfa mosaic virus (AMV RNA 4), (Jobling, S. A., and Gehrke, L., (1987) Nature, 325: 622-625; tobacco mosaic virus leader (TMV), (Gallie et al. (1989) Molecular Biology of RNA, pages 237-256, Gallie et al. (1987) Nucl. Acids Res. 15: 3257-3273; maize chlorotic mottle virus leader (MCMV) (Lornmel, S. A. et al. (1991) Virology, 81: 382-385). See also, Della-Cioppa et al. (1987) Plant Physiology, 84: 965-968; and endogenous maize 5′ untranslated sequences. Other methods known to enhance translation can also be utilized, for example, introns, and the like.

The expression cassettes may contain one or more than one gene or nucleic acid sequence to be transferred and expressed in the transformed plant. Thus, each nucleic acid sequence will be operably linked to 5′ and 3′ regulatory sequences. Alternatively, multiple expression cassettes may be provided.

The morphogenic genes and/or genes/polynucleotides of interest introduced into an explant by the disclosed methods can be operably linked to a suitable promoter. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as from Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissues are referred to as “tissue specific”. A “cell type” specific promoter primarily drives expression in certain cell types in one or more organs, for example, vascular cells in roots or leaves. Tissue specific, tissue preferred, cell type specific, and inducible promoters constitute the class of “non-constitutive” promoters.

An “inducible” or “repressible” promoter can be a promoter which is under either environmental or exogenous control. Examples of environmental conditions that may affect transcription by inducible promoters include anaerobic conditions, or certain chemicals, or the presence of light. Alternatively, exogenous control of an inducible or repressible promoter can be affected by providing a suitable chemical or other agent that via interaction with target polypeptides result in induction or repression of the promoter. Inducible promoters include heat-inducible promoters, estradiol-responsive promoters, chemical inducible promoters, and the like. Pathogen inducible promoters include those from pathogenesis-related proteins (PR proteins), which are induced following infection by a pathogen; e. g., PR proteins, SAR proteins, beta-1,3-glucanase, chitinase, etc. See, for example, Redolfi et al. (1983) Neth. J. Plant Pathol. 89: 245-254; Uknes et al. (1992) The Plant Cell 4: 645-656; and Van Loon (1985) Plant Mol. Virol. 4: 111-116. Inducible promoters useful in the present methods include GLB1, OLE, LTP2, XVE and heat shock inducible promoters HSP17.7, HSP26, HSP18A, AT-HSP811, AT-HSP811L, and GM-HSP173B.

A chemically-inducible promoter can be repressed by the tetraycline repressor (TETR), the ethametsulfuron repressor (ESR), or the chlorsulfuron repressor (CR), and de-repression occurs upon addition of tetracycline-related or sulfonylurea ligands. The repressor can be TETR and the tetracycline-related ligand is doxycycline or anhydrotetracycline. (Gatz, C., Frohberg, C. and Wendenburg, R. (1992) Stringent repression and homogeneous de-repression by tetracycline of a modified CaMV 35S promoter in intact transgenic tobacco plants, Plant J. 2, 397-404). Alternatively, the repressor can be ESR and the sulfonylurea ligand is ethametsulfuron, chlorsulfuron, metsulfuron-methyl, sulfometuron methyl, chlorimuron ethyl, nicosulfuron, primisulfuron, tribenuron, sulfosulfuron, trifloxysulfuron, foramsulfuron, iodosulfuron, prosulfuron, thifensulfuron, rimsulfuron, mesosulfuron, or halosulfuron (US20110287936 incorporated herein by reference in its entirety). If the repressor is CR, the CR ligand is chlorsulfuron. See, U.S. Pat. No. 8,580,556 incorporated herin by reference in its entirety.

A “constitutive” promoter is a promoter which is active under most conditions. Promoters useful in the present disclosure include those disclosed in WO2017/112006 and those disclosed in U.S. Provisional Application 62/562,663. Constitutive promoters for use in expression of genes in plants are known in the art. Such promoters include, but are not limited to 35S promoter of cauliflower mosaic virus (Depicker et al. (1982) Mol. Appl. Genet. 1: 561-573; Odell et al. (1985) Nature 313: 810-812), ubiquitin promoter (Christensen et al. (1992) Plant Mol. Biol. 18: 675-689), promoters from genes such as ribulose bisphosphate carboxylase (De Almeida et al. (1989) Mol. Gen. Genet. 218: 78-98), actin (McElroy et al. (1990) Plant J. 2: 163-171), histone, DnaJ (Baszczynski et al. (1997) Maydica 42: 189-201), and the like. In various aspects, constitutive promoters useful in the methods of the disclosure include UBI, LLDAV, EVCV, DMMV, BSV (AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, and ZM-ADF PRO (ALT2) promoters.

Promoters useful in the present disclosure include those disclosed in US Patent Application Publication Number 2017/0121722, U.S. Pat. No. 8,710,206, U.S. Provisional Patent Applications 62/562,663 and 62/641,725, and WO2017112006 all of which are incorporated herein by reference in their entireties.

As used herein, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. In the context of the methods of the disclosure a post-transcriptional regulatory element may include elements that are active following transcription initiation, for example translational and transcriptional enhancers, translational and transcriptional repressors and mRNA stability determinants.

A “heterologous nucleotide sequence”, “heterologous polynucleotide of interest”, or “heterologous polynucleotide” as used throughout the disclosure, is a sequence that is not naturally occurring with or operably linked to a promoter. While this nucleotide sequence is heterologous to the promoter sequence, it may be homologous or native or heterologous or foreign to the plant host. Likewise, the promoter sequence may be homologous or native or heterologous or foreign to the plant host and/or the polynucleotide of interest.

The DNA constructs and expression cassettes useful in the methods of the disclosure can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified to increase translation of the mRNA. It is recognized that to increase transcription levels enhancers may be utilized in combination with promoter regions. It is recognized that to increase transcription levels, enhancers may be utilized in combination with promoter regions. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

It is recognized that sequences useful in the methods of the disclosure may be used with their native coding sequences thereby resulting in a change in phenotype of the transformed plant. The morphogenic genes and genes of interest disclosed herein, as well as variants and fragments thereof, are useful in the methods of the disclosure for the genetic manipulation of any plant. The term “operably linked” means that the transcription or translation of a heterologous nucleotide sequence is under the influence of a promoter sequence.

In one aspect of the disclosure, expression cassettes comprise a transcriptional initiation region or variants or fragments thereof, operably linked to a morphogenic gene and/or a heterologous nucleotide sequence. Such expression cassettes can be provided with a plurality of restriction sites for insertion of the nucleotide sequence to be under the transcriptional regulation of the regulatory regions. The expression cassettes may additionally contain selectable marker genes as well as 3′ termination regions.

The expression cassettes can include, in the 5′-3′ direction of transcription, a transcriptional initiation region (i.e., a promoter, or variant or fragment thereof), a translational initiation region, a morphogenic gene and/or a heterologous nucleotide sequence of interest, a translational termination region and optionally, a transcriptional termination region functional in the host organism. The regulatory regions (i.e., promoters, transcriptional regulatory regions, and translational termination regions), the morphogenic gene and/or the polynucleotide of interest useful in the methods of the disclosure may be native/analogous to the host cell or to each other. Alternatively, the regulatory regions, morphogenic gene and/or the polynucleotide of interest may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus or the promoter is not the native promoter for the operably linked polynucleotide.

The termination region may be native with the transcriptional initiation region, may be native with the operably linked morphogenic gen and/or may be native with the operably linked DNA sequence of interest, may be native with the plant host, or may be derived from another source (i.e., foreign or heterologous to the promoter, the morphogenic gene and/or the DNA sequence being expressed, the plant host, or any combination thereof). Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639, herein incorporated by reference in their entirety.

The expression cassette comprising a promoter operably linked to a morphogenic gene and/or optionally further operably linked to a heterologous nucleotide sequence, a heterologous polynucleotide of interest, a heterologous polynucleotide nucleotide, or a sequence of interest can be used to transform any plant. Alternatively, a heterologous polynucleotide of interest, a heterologous polynucleotide nucleotide, or a sequence of interest operably linked to a promoter can be on a separate expression cassette positioned outside of the transfer-DNA. In this manner, genetically modified plants, plant cells, plant tissue, seed, root and the like can be obtained. The expression cassette comprising the sequences of the present disclosure may also contain at least one additional nucleotide sequence for a gene, heterologous nucleotide sequence, heterologous polynucleotide of interest, or heterologous polynucleotide to be cotransformed into the organism. Alternatively, the additional nucleotide sequence(s) can be provided on another expression cassette.

Where appropriate, the nucleotide sequences whose expression is to be under the control a promoter sequence and any additional nucleotide sequence(s) may be optimized for increased expression in the transformed plant. That is, these nucleotide sequences can be synthesized using plant preferred codons for improved expression. See, for example, Campbell and Gowri, (1990) Plant Physiol. 92:1-11, herein incorporated by reference in its entirety, for a discussion of host-preferred codon usage. Methods are available in the art for synthesizing plant-preferred genes. See, for example, U.S. Pat. Nos. 5,380,831, 5,436,391 and Murray, et al., (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of a heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes useful in the methods of the disclosure may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize AdhI intron (Kyozuka, et al., (1991) Mol. Gen. Genet. 228:40-48; Kyozuka, et al., (1990) Maydica 35:353-357) and the like, herein incorporated by reference in their entirety.

In preparing expression cassettes useful in the methods of the disclosure, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

As used herein, “vector” refers to a DNA molecule such as a plasmid, cosmid or bacterial phage for introducing a nucleotide construct, for example, an expression cassette, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

Cells that have been transformed may be grown into plants in accordance with conventional ways. See, for example, McCormick, et al., (1986) Plant Cell Reports 5:81-84, herein incorporated by reference in its entirety. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct useful in the methods of the disclosure, for example, an expression cassette useful in the methods of the disclosure, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant produced by the methods of the disclosure containing a desired polynucleotide of interest is cultivated using methods well known to one skilled in the art.

Methods are known in the art for the targeted insertion of a polynucleotide at a specific location in the plant genome. The insertion of the polynucleotide at a desired genomic location is achieved using a site-specific recombination system. See, for example, WO99/25821, WO99/25854, WO99/25840, WO99/25855 and WO99/25853, all of which are herein incorporated by reference in their entirety. Briefly, a polynucleotide of interest can be contained in transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The disclosed methods can be used to introduce into explants polynucleotides that are useful to target a specific site for modification in the genome of a plant derived from the explant. Site specific modifications that can be introduced with the disclosed methods include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence of the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

Cas gene includes a gene that is generally coupled, associated or close to or in the vicinity of flanking CRISPR loci. The terms “Cas gene” and “CRISPR-associated (Cas) gene” are used interchangeably herein. A comprehensive review of the Cas protein family is presented in Haft et al. (2005) Computational Biology, PLoS Comput Biol 1 (6): e60. doi:10.1371/journal.pcbi.0010060.

In addition to the four initially described gene families, an additional 41 CRISPR-associated (Cas) gene families have been described in US Patent Application Publication Number 2015/0059010, which is incorporated herein by reference. This reference shows that CRISPR systems belong to different classes, with different repeat patterns, sets of genes, and species ranges. The number of Cas genes at a given CRISPR locus can vary between species. Cas endonuclease relates to a Cas protein encoded by a Cas gene, wherein the Cas protein is capable of introducing a double strand break into a DNA target sequence. The Cas endonuclease is guided by the guide polynucleotide to recognize and optionally introduce a double strand break at a specific target site into the genome of a cell. As used herein, the term “guide polynucleotide/Cas endonuclease system” includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide nucleotide, but only if the correct protospacer-adjacent motif (PAM) is approximately oriented at the 3′ end of the target sequence (see FIG. 2A and FIG. 2B of US Patent Application Publication Number 2015/0059010).

In an aspect, the Cas endonuclease gene is a Cas9 endonuclease, such as, but not limited to, Cas9 genes listed in SEQ ID NOs: 462, 474, 489, 494, 499, 505, and 518 of WO2007/025097, published Mar. 1, 2007, and incorporated herein by reference. In another aspect, the Cas endonuclease gene is plant, maize or soybean optimized Cas9 endonuclease, such as, but not limited to those shown in FIG. 1A of US Patent Application Publication Number 2015/0059010.

In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

In an aspect, the Cas endonuclease gene is a Cas9 endonuclease gene of SEQ ID NO:1, 124, 212, 213, 214, 215, 216, 193 or nucleotides 2037-6329 of SEQ ID NO:5, or any functional fragment or variant thereof, of US Patent Application Publication Number 2015/0059010.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG which can in principle be targeted.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

A “Dead-CAS9” (dCAS9) as used herein, is used to supply a transcriptional repressor domain. The dCAS9 has been mutated so that can no longer cut DNA. The dCASO can still bind when guided to a sequence by the gRNA and can also be fused to repressor elements (see Gilbert et al., Cell 2013 Jul. 18; 154(2): 442-451, Kiani et al., 2015 November Nature Methods Vol. 12 No. 11: 1051-1054). The dCAS9 fused to the repressor element, as described herein, is abbreviated to dCAS9˜REP, where the repressor element (REP) can be any of the known repressor motifs that have been characterized in plants (see Kagale and Rozxadowski, 20010 Plant Signaling & Behavior 5:6, 691-694 for review). An expressed guide RNA (gRNA) binds to the dCAS9˜REP protein and targets the binding of the dCAS9-REP fusion protein to a specific predetermined nucleotide sequence within a promoter (a promoter within the T-DNA). For example, if this is expressed Beyond-the Border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the Beyond-the-Border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9-REP (Urritia et al., 2003, Genome Biol. 4:231) as described above.

Bacteria and archaea have evolved adaptive immune defenses termed clustered regularly interspaced short palindromic repeats (CRISPR)/CRISPR-associated (Cas) systems that use short RNA to direct degradation of foreign nucleic acids ((WO2007/025097 published Mar. 1, 2007). The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In an aspect, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In an aspect, the size of the fragment of the cRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides.

In an aspect, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In an aspect, the RNA that guides the RNA Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In an aspect, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide nucleotide” (when composed of a contiguous stretch of RNA nucleotides) or “single guide DNA” (when composed of a contiguous stretch of DNA nucleotides) or “single guide nucleotide-DNA” (when composed of a combination of RNA and DNA nucleotides). In an aspect of the disclosure, the single guide nucleotide comprises a cRNA or cRNA fragment and a tracrRNA or tracrRNA fragment of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In an aspect, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In an aspect, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another aspect, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the disclosure the variable target domain is 12, 13, 14, 15, 16, 17, 18, 19, 20, 21, 22, 23, 24, 25, 26, 27, 28, 29 or 30 nucleotides in length.

In an aspect of the disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that that is specifically recognized and/or bound by a double-strand break inducing agent such as a LIG3-4 endonuclease (US Patent Application Publication Number 2009/0133152) or a MS26++ meganuclease (US Patent Application Publication Number 2014/0020131).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods can be used to introduce into plants polynucleotides useful for gene suppression of a target gene in a plant. Reduction of the activity of specific genes (also known as gene silencing, or gene suppression) is desirable for several aspects of genetic engineering in plants. Many techniques for gene silencing are well known to one of skill in the art, including but not limited to antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12: 883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; Javier (2003) Nature 425:257-263; and, Montgomery et al. (1998) Proc. Natl. Acad. Sci. USA 95:15502-15507), virus-induced gene silencing (Burton, et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; and WO 98/53083); ribozymes (Steinecke et al. (1992) EMBO J. 11:1525; U.S. Pat. No. 4,987,071; and, Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); artificial micro RNAs (U.S. Pat. No. 8,106,180; Schwab et al. (2006) Plant Cell 18:1121-1133); and other methods or combinations of the above methods known to those of skill in the art.

In an aspect, the disclosed methods can be used to introduce into plants polynucleotides useful for the targeted integration of nucleotide sequences into a plant. For example, the disclosed methods can be used to introduce transfer cassettes comprising nucleotide sequences of interest flanked by non-identical recombination sites are used to transform a plant comprising a target site. In an aspect, the target site contains at least a set of non-identical recombination sites corresponding to those on the transfer cassette. The exchange of the nucleotide sequences flanked by the recombination sites is affected by a recombinase. Thus, the disclosed methods can be used for the introduction of transfer cassettes for targeted integration of nucleotide sequences, wherein the transfer cassettes which are flanked by non-identical recombination sites recognized by a recombinase that recognizes and implements recombination at the nonidentical recombination sites. Accordingly, the disclosed methods and composition can be used to improve efficiency and speed of development of plants containing non-identical recombination sites.

Thus, the disclosed methods can further comprise methods for the directional, targeted integration of exogenous nucleotides into a transformed plant. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the transfer cassette.

It is recognized that the transformed plant prepared in this manner may comprise multiple target sites; i. e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

Examples of recombination sites for use in the disclosed method are known in the art and include FRT sites (See, for example, Schlake and Bode (1994) Biochemistry 33: 12746-12751; Huang et al. (1991) Nucleic Acids Research 19: 443-448; Paul D. Sadowski (1995) In Progress in Nucleic Acid Research and Molecular Biology vol. 51, pp. 53-91; Michael M. Cox (1989) In Mobile DNA, Berg and Howe (eds) American Society of Microbiology, Washington D. C., pp. 116-670; Dixon et al. (1995) 18: 449-458; Umlauf and Cox (1988) The EMBO Journal 7: 1845-1852; Buchholz et al. (1996) Nucleic Acids Research 24: 3118-3119; Kilby et al. (1993) Trends Genet. 9: 413-421: Rossant and Geagy (1995) Nat. Med. 1: 592-594; Albert et al. (1995) The Plant J. 7: 649-659: Bayley et al. (1992) Plant Mol. Biol. 18: 353-361; Odell et al. (1990) Mol. Gen. Genet. 223: 369-378; and Dale and Ow (1991) Proc. Natl. Acad. Sci. USA 88: 10558-105620; all of which are herein incorporated by reference.); Lox (Albert et al. (1995) Plant J. 7: 649-659; Qui et al. (1994) Proc. Natl. Acad. Sci. USA 91: 1706-1710; Stuurman et al. (1996) Plant Mol. Biol. 32: 901-913; Odell et al. (1990) Mol. Gen. Gevet. 223: 369-378; Dale et al. (1990) Gene 91: 79-85; and Bayley et al. (1992) Plant Mol. Biol. 18: 353-361.) The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et al. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the disclosure prevent or greatly suppress recombination between the two flanking recombination sites and excision of the nucleotide sequence contained therein. Accordingly, it is recognized that any suitable non-identical recombination sites may be utilized in the disclosure, including FRT and mutant FRT sites, FRT and lox sites, lox and mutant lox sites, as well as other recombination sites known in the art.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A. 80: 4223-4227. The FLP recombinase for use in the disclosure may be that derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. The Cre recombinase is known in the art. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702. All of which are herein incorporated by reference. Such Cre sequence may also be synthesized using plant preferred codons.

Where appropriate, the nucleotide sequences to be inserted in the plant genome may be optimized for increased expression in the transformed plant. Where mammalian, yeast, or bacterial genes are used in the disclosure, they can be synthesized using plant preferred codons for improved expression. It is recognized that for expression in monocots, dicot genes can also be synthesized using monocot preferred codons. Methods are available in the art for synthesizing plant preferred genes. See, for example, U.S. Pat. Nos 5,380,831,5,436,391, and Murray et al. (1989) Nucleic Acids Res. 17: 477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host and can be used in the disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses novel FLP recombination target sites (FRT). The FRT has been identified as a minimal sequence comprising two 13 base pair repeats, separated by an eight 8 base spacer, as follows: 5′-GAAGTTCCTATTC[TCTAGAAA]GTATAGGAACTTC-3′ (SEQ ID NO: 45) wherein the nucleotides within the brackets indicate the spacer region. The nucleotides in the spacer region can be replaced with a combination of nucleotides, so long as the two 13-base repeats are separated by eight nucleotides. It appears that the actual nucleotide sequence of the spacer is not critical; however, for the practice of the disclosure, some substitutions of nucleotides in the space region may work better than others. The eight-base pair spacer is involved in DNA-DNA pairing during strand exchange. The asymmetry of the region determines the direction of site alignment in the recombination event, which will subsequently lead to either inversion or excision. As indicated above, most of the spacer can be mutated without a loss of function. See, for example, Schlake and Bode (1994) Biochemistry 33: 12746-12751, herein incorporated by reference.

Novel FRT mutant sites can be used in the practice of the disclosed methods. Such mutant sites may be constructed by PCR-based mutagenesis. Although mutant FRT sites are known (see SEQ ID Nos 2, 3, 4 and 5 of WO1999/025821), it is recognized that other mutant FRT sites may be used in the practice of the disclosure. The present disclosure is not the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

As discussed above, bringing genomic DNA containing a target site with non-identical recombination sites together with a vector containing a transfer cassette with corresponding non-identical recombination sites, in the presence of the recombinase, results in recombination. The nucleotide sequence of the transfer cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the transfer cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a transfer cassette comprising a coding region, expression of the coding region will occur upon integration of the transfer cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing transfer cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

The disclosed methods also provide for means to combine multiple cassettes at one location within the genome. Recombination sites may be added or deleted at target sites within the genome.

Any means known in the art for bringing the three components of the system together may be used in the disclosure. For example, a plant can be stably transformed to harbor the target site in its genome. The recombinase may be transiently expressed or provided. Alternatively, a nucleotide sequence capable of expressing the recombinase may be stably integrated into the genome of the plant. In the presence of the corresponding target site and the recombinase, the transfer cassette, flanked by corresponding non-identical recombination sites, is inserted into the transformed plant's genome.

Alternatively, the components of the system may be brought together by sexually crossing transformed plants. In this aspect, a transformed plant, parent one, containing a target site integrated in its genome can be sexually crossed with a second plant, parent two, that has been genetically transformed with a transfer cassette containing flanking non-identical recombination sites, which correspond to those in plant one. Either plant one or plant two contains within its genome a nucleotide sequence expressing recombinase. The recombinase may be under the control of a constitutive or inducible promoter. In this manner, expression of recombinase and subsequent activity at the recombination sites can be controlled.

The disclosed methods are useful in targeting the integration of transferred nucleotide sequences to a specific chromosomal site. The nucleotide sequence may encode any nucleotide sequence of interest. Particular genes of interest include those which provide a readily analyzable functional feature to the host cell and/or organism, such as marker genes, as well as other genes that alter the phenotype of the recipient cells, and the like. Thus, genes effecting plant growth, height, susceptibility to disease, insects, nutritional value, and the like may be utilized in the disclosure. The nucleotide sequence also may encode an ‘antisense’ sequence to turn off or modify gene expression.

It is recognized that the nucleotide sequences will be utilized in a functional expression unit or cassette. By functional expression unit or cassette is intended, the nucleotide sequence of interest with a functional promoter, and in most instances a termination region. There are various ways to achieve the functional expression unit within the practice of the disclosure. In one aspect of the disclosure, the nucleic acid of interest is transferred or inserted into the genome as a functional expression unit.

Alternatively, the nucleotide sequence may be inserted into a site within the genome which is 3′ to a promoter region. In this latter instance, the insertion of the coding sequence 3′ to the promoter region is such that a functional expression unit is achieved upon integration. For convenience, for expression in plants, the nucleic acid encoding target sites and the transfer cassettes, including the nucleotide sequences of interest, can be contained within expression cassettes. The expression cassette will comprise a transcriptional initiation region, or promoter, operably linked to the nucleic acid encoding the peptide of interest. Such an expression cassette is provided with a plurality of restriction sites for insertion of the gene or genes of interest to be under the transcriptional regulation of the regulatory regions.

### EXPERIMENTAL

### Example 1: Agrobacterium-Mediated Transformation of Corn

### A. Preparation of Agrobacterium Master Plate.

Agrobacterium tumefaciens harboring a binary donor vector is streaked out from a −80° C. frozen aliquot onto solid 12R medium and cultured at 28° C. in the dark for 2-3 days to make a master plate.

### B. Growing Agrobacterium on Solid Medium.

A single colony or multiple colonies of Agrobacterium are picked from the master plate and streaked onto a second plate containing 810K medium and incubated at 28° C. in the dark overnight.

Agrobacterium infection medium (700A; 5 ml) and 100 mM 3′-5′-Dimethoxy-4′-hydroxyacetophenone (acetosyringone; 5 μL) are added to a 14 ml conical tube in a hood. About 3 full loops of Agrobacterium from the second plate are suspended in the tube and the tube is then vortexed to make an even suspension. The suspension (1 ml) is transferred to a spectrophotometer tube and the optical density (550 nm) of the suspension is adjusted to a reading of about 0.35-1.0. The Agrobacterium concentration is approximately 0.5 to 2.0×10⁹ cfu/mL. The final Agrobacterium suspension is aliquoted into 2 mL microcentrifuge tubes, each containing about 1 mL of the suspension. The suspensions are then used as soon as possible.

### C. Growing Agrobacterium on Liquid Medium.

Alternatively, Agrobacterium can be prepared for transformation by growing in liquid medium. One day before infection, a 125 ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate is suspended into the flasks and placed on an orbital shaker set at 200 rpm and incubated at 28° C. overnight. The Agrobacterium culture is centrifuged at 5000 rpm for 10 min. The supernatant is removed and the Agrobacterium infection medium (700A) with acetosyringone solution is added. The bacteria are resuspended by vortex and the optical density (550 nm) of the Agrobacterium suspension is adjusted to a reading of about 0.35 to 2.0.

### D. Maize Transformation.

Ears of a maize (Zea mays L.) cultivar are surface-sterilized for 15-20 min in 20% (v/v) bleach (5.25% sodium hypochlorite) plus 1 drop of Tween 20 followed by 3 washes in sterile water. Immature embryos (IEs) are isolated from ears and placed in 2 ml of the Agrobacterium infection medium (700A) with acetosyringone solution. The optimal size of the embryos varies based on the inbred, but for transformation with WUS2 and ODP2 a wide size range of immature embryo sizes can be used. The Agrobacterium infection medium (810K) is drawn off and 1 ml of the Agrobacterium suspension is added to the embryos and the tube is vortexed for 5-10 sec. The microfuge tube is allowed to stand for 5 min in the hood. The suspension of Agrobacterium and embryos is poured onto 710I (or 562V) co-cultivation medium (see Table 10). Any embryos left in the tube are transferred to the plate using a sterile spatula. The Agrobacterium suspension is drawn off and the embryos placed axis side down on the media. The plate is incubated in the dark at 21° C. for 1-3 days of co-cultivation.

Embryos are transferred to resting medium (605T medium) without selection (see Table 10). Three to 7 days later, the embryos are transferred to maturation medium (289Q medium) supplemented with a selective agent (see Table 10).

### Example 2: Particle Bombardment for Site-Specific Integration

Pioneer inbred PH184C (disclosed in U.S. Pat. No. 8,445,763 incorporated herein by reference in its entirety) that contains in chromosome-1 a pre-integrated Site-Specific Integration target site (Chrom-1 target site) composed of UBI PRO:FRT1:NPTII::PINII TERM+FRT87 is used. Prior to bombardment, 10-12 DAP (days after pollination) immature embryos are isolated from ears of Pioneer inbred PH184C and placed on 605J culture medium plus 16% sucrose for three hours to plasmolyze the scutellar cells.

Four plasmids are typically used for each particle bombardment:
1) a donor plasmid (100 ng/μ1) containing a FRT-flanked donor cassette for Recombinase-Mediated Cassette Exchange, for example a plasmid containing FRT1:PMI:PINII TERM+UBI1ZM PRO::DS-RED2::PINII TERM+FRT87 (PHP0004, SEQ ID NO: 17);
2) a plasmid (2.5 ng/μ1) containing the expression cassette UBI1ZM PRO::FLPm::PINII TERM (PHP5096, SEQ ID NO: 18);
3) a plasmid (10 ng/μ1) containing the expression cassette ZM-PLTP PRO::ZM-ODP2::OS-T28 TERM+FMV & PCSV ENHANCERS (PHP89030, SEQ ID NO: 19); and
4) a plasmid (5 ng/μ1) containing the expression cassette ZM-PLTP PRO::ZM-WUS2::IN2-1 TERM (PHP89179, SEQ ID NO: 20).

To attach the DNA to 0.6 μm gold particles, the four plasmids are mixed by adding 10 μl of each plasmid together in a low-binding microfuge tube (Sorenson Bioscience 39640T) for a total of 40 μl. To this suspension, 50 μl of 0.6 μm gold particles (30 μg/μ1) and 1.0 μl of Transit 20/20 (Cat No MIR5404, Minis Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all remaining ethanol. Particle bombardment is performed using a Biolistics PDF-1000, at 28 inches of Mercury using a 200 PSI rupture disc. After particle bombardment, the immature embryos are selected on 605J medium modified to contain 12.5 g/l mannose and 5 g/l maltose and no sucrose. After 10-12 weeks on selection, plantlets are regenerated and analyzed using qPCR. Co-delivery of PLTP::ODP2 (PHP89030, SEQ ID NO: 19) and PLTP::WUS2 (PHP89179, SEQ ID NO: 20) along with the SSI components (Donor DNA (PHP0004, SEQ ID NO: 17)+UBI1ZM PRO::FLPm::PINII TERM (PHP5096, SEQ ID NO: 18)) produces site-specific integration of the donor fragment into the Chrom-1 target site at rates of 4-7% relative to the number of bombarded immature embryos.

### Example 3: Rapid Recovery of Streptomycin Resistant Corn Plants

### A. Determining an Effective Spectinomycin and Streptomycin Concentration for Inhibition of Somatic Embryo Germination and Growth of Non-Transgenic Maize Plants.

To determine the effective amount of streptomycin to use in selection of transgenic maize plants Pioneer inbreds PHR03, GR84Z, and ED85E were subjected to experimental conditions that included mock infection with a disarmed strain of Agrobacterium, followed by culturing in media supplemented with different concentrations of spectinomycin and streptomycin. Two to three ears for each genotype were transformed and embryos evenly split between nine treatments. For transformation control embryos were transformed and then cultured on media without selection. For detecting the direct effect of the selective agent on an embryo, ten to fifteen embryos per ear were not transformed and were cultured on similar media as other treatments which served as the positive control in the experiments. We tested four different concentrations of two different selective agents namely, spectinomycin and streptomycin, at concentrations of 25, 50, 100 and 150 mg/L to establish a kill curve for each antibiotic.

For PHR03 transformation, the same media was used for all treatments with various concentrations of selective agent, namely, cocultured for two to four days at 21° C. on 710I medium, moved to 13152C medium for resting for seven to ten days at 28° C. in dark, moved to 13152C medium containing a respective concentration of selective agent for three weeks at 28° C. in dim light, moved to 13329B medium (MS salts and vitamins (T Murashige and F Skoog (1962) Physiol Plant 15:473-497), 0.1 g/l myo-inositol, 0.5 mg/l zeatin, 1.25 mg/l cupric sulfate, 0.7 g/l proline, 600 g/l sucrose, 1 gm/l IAA, 0.1 μm ABA, 10 mg/l meropenem, 1 mg/l BAP, and 8 g/l Sigma agar, pH 5.6) containing the respective selective agent for maturation for two to three weeks at 28° C. in dim light. Then shoots were transferred to light for two to three days and the shoot color was monitored and photographed for visual phenotyping (data not shown).

For ED85E and GR84Z transformations, the same media was used for all treatments with various concentrations of selective agent, namely, cocultured for one day at 21° C. on 562V medium, moved to 605B medium for resting for eleven to fourteen days at 28° C. in the dark, moved to 605B medium containing the respective concentration of selective agent for three weeks at 28° C. in dim light, moved to 13329B medium containing the respective selective agent for maturation for teo to three weeks at 28° C. in dim light. Then shoots were transferred to light for two to three days and the shoot color was monitored and photographed for visual phenotyping (data not shown).

No apparent differences in the callus morphology was observed in the selection media with antibiotics compared to the transformation control material and non-transformed controls (data not shown).

Further kill curve experiments with Pioneer inbred PHR03 were conducted to detect the effect of spectinomycin and streptomycin on maize transformation at the maturation stage. Maturation media supplemented with spectinomycin grew normally and produced shoots like on the media without spectinomycin. However, maturation media supplemented with streptomycin (25 mg/L) resulted in production of events containing a mixture of green and bleached phenotypes, suggesting the potency of the antibiotic as a selectable agent for maize transformation. With higher concentrations of streptomycin (50 mg/L and above), most of the shoots produced were bleached and higher concentrations of streptomycin (100-150 mg/L) adversely affected shoot formation and regeneration (data not shown). Two additional Pioneer corn inbreds, ED85E and GR84Z were also subjected to streptomycin selection at maturation stage, resulting in bleached shoots at 25 mg/L and shoot development was affected at 100-125 mg/L. For inbreds PHR03, ED85E and GR84Z streptomycin was a better selection agent for maize transformation than spectinomycin.

B. Co-Bombardment of SPCN Expression Cassette Along with PLTP::ODP2 and Axig1::WUS Cassettes Resulted in Rapid Selection of Streptomycin-Resistant Maize T0 Plantlets.

To evaluate the effect of the maize optimized SPCN gene on immature embryo transformation, we carried out co-bomambered experiments in maize inbred PHR03. Immature embryos were harvested from Pioneer inbred PHR03 ten to twelve days after pollination. The immature embryos were placed on high-osmotic medium to induce plasmolysis. Meanwhile, three plasmids PHP91619 (containing PRO::UBI1ZM 5′ UTR::UBI1ZM INTRON1:FRT1:CTP::SPCN::SB-UBI TERM, SEQ ID NO: 3), PHP75799 (containing ZM-PLTP PRO::ZM-PLTP 5′ UTR::ZM-ODP2::OS-T28 TERM, SEQ ID NO: 4), and PHP76976 (containing ZM-AXIG1 PRO::ZM-WUS2::IN1-2 TERM, SEQ ID NO: 5) were individually precipitated onto 0.6 μM gold particles and introduced into the scutellar cells of the immature embryos. As a control, just PHP75799 (SEQ ID NO: 4) and PHP76976 (SEQ ID NO: 5) were used, with no SPCN-containing plasmid. After particle bombardment and culturing the zygotic immature embryos on the media series described in A above, with either 50 mg/l or 100 mg/l streptomycin, no streptomycin-resistant, green plantlets were recovered in the control treatments (PHP75799 (SEQ ID NO: 4) and PHP76976 (SEQ ID NO: 5)). However, in the treatment in which all three plasmids were introduced (PHP91619 (SEQ ID NO: 3), PHP75799 (SEQ ID NO: 4), and PHP76976 (SEQ ID NO: 5)), streptomycin-resistant, green T0 plantlets were produced at a frequency of between 10-30% relative to the number of bombarded immature embryos.

The regenerated plants with green shoots and bleached shoots were collected for molecular analysis by qPCR. Any plant that was positive for the SPCN gene was considered a transgenic event. The molecular data is presented in Table 4. We identified five (5) SPCN transgenic events from the total of fourteen (14) green looking plants, while four (4) out of the six (6) bleached plants were also positive for SPCN marker gene. The maize-codon-optimized SPCN gene (SEQ ID NO: 11) was efficacious for conferring resistance to streptomycin in maize.

TABLE 4
Molecular event data of the transgenic maize plants transformed
with the SPCN gene by particle bombardment.

| | SB | ODP-T28 | ZM-WUS2 |
|---|---|---|---|
| Plant | UBI_TERM PCR | TERM | IN2 TERM |
| Phenotype | (SPCN) (+/−) | Copy # | Copy # |
| Green | − | 0 | 0 |
| Bleached | − | 0 | 0 |
| Green | + | 2 | 2 |
| Green | − | 0 | 0 |
| Green | + | 2 | 1 |
| Green | − | 0 | 0 |
| Green | − | 0 | 0 |
| Green | − | 0 | 0 |
| Green | − | 0 | 0 |
| Green | + | 1 | 1 |
| Green | − | 0 | 0 |
| Bleached | + | 1 | 1 |
| Bleached | + | 3 | 1 |
| Green | − | 0 | 0 |
| Bleached | + | 0 | 0 |
| Green | + | 0 | 0 |
| Green | + | 0 | 0 |
| Bleached | + | 0 | 0 |
| Green | − | 0 | 0 |
| Bleached | − | 0 | 0 |

C. Agrobacterium Transformation of SPCN Expression Cassette Along with PLTP::WUS Cassettes Resulted in Efficacious Rapid Selection of Streptomycin-Resistant Maize T0 Plantlets.

Immature embryos of Pioneer inbred PHR03 were isolated from immature ears (ten to twelve days after pollination) and were transformed with an Agrobacterium strain carrying the SPCN gene. Specifically, the immature embryos were transformed with Agrobacterium strain LBA4404 THY- (disclosed in U.S. Pat. No. 8,334,429 and incorporated herein in by reference its entirety) containing PHP71539 (SEQ ID NO: 1) (disclosed in U.S. Patent Appln. No. US20190078106 and incorporated herein by reference in its entirety). A binary plasmid PHP92307 (SEQ ID NO: 32) containing the T-DNA expression cassette RB+LOXP+PLTP:WUS:IN2-1 TERM+ZMHSP17.7:MO-CRE:PINII TERM+UBI1ZMPRO:NPTII:SB-UBI TERM+UBI1ZM PRO-FRT1 FRT1:CTP::SPCN::SB-UBI TERM+LB) was used to evaluate the selection of NPTII transgenic plants on a medium supplemented with G418 (control) and the selection of SPCN transgenic plants on a medium supplemented with Streptomycin. Two different concentrations of streptomycin (50 and 100 mg/1) were tested for selecting plants that are resistant to a streptomycin selective agent. After infection with Agrobacterium the immature embryos were co-cultured for two to four days at 21° C. on 710I medium, then were transferred to13152C medium (resting medium with no selection) for seven to ten days at 28° C. in the dark, then moved onto 13152C medium (with and without the respective concentrations of streptomycin) for three weeks at 28° C. in dim light, and were then transferred to 13329B medium (with and without the respective concentrations of streptomycin) for somatic embryo maturation for two to three weeks at 28° C. in dim light. Then the shoots were transferred to the light for two to three days and their vigor and leaf color was evaluated (data not shown).

Plantlets on 50 mg/L streptomycin displayed a mixture of green and bleached (white) leaves, on 100 mg/L all of the shoots were bleached. The green and pale green shoots were regenerated in <50 days. These shoots were subjected to molecular data analysis by qPCR. Eighty-one plants were regenerated and twenty-nine of those plants were identified as transgenic, a 35% transgenic plant recovery. The molecular data of the transgenic plants are presented in Table 5. This data showed streptomycin concentrations between about 50 mg/L and about 100 mg/L were efficacious for selection of transgenic plants expressing the maize-optimized SPCN gene.

TABLE 5
PHR03 molecular event data of maize plants transformed with an
Agrobacterium containing the binary vector carrying the SPCN gene

| | SB UBI_TERM | | | |
|---|---|---|---|---|
| Plant | PCR (SPCN) | ZM-WUS2 IN2 | MO CRE PCR | BACKBONE PCR |
| phenotype | Copy # | TERM Copy # | (+/−) | (+/−) |
| Green | 2 | 1 | + | + |
| Green | 1 | 0 | − | − |
| Green | 1 | 1 | + | − |
| Green | 1 | 0 | − | − |
| Green | 1 | UNDETERMINED | + | − |
| Green | 1 | 1 | + | − |
| Green | 1 | UNDETERMINED | + | − |
| Green | 1 | 0 | − | − |
| Green | 2 | 0 | − | − |
| Green | 1 | 1 | + | − |
| Green | 3 | 2 | + | + |
| Green | 1 | 0 | UNDETERMINED | − |
| Green | 1 | 0 | − | − |
| Green | 1 | 0 | − | − |
| Green | 1 | UNDETERMINED | UNDETERMINED | − |

D. SPCN Expression Cassette Along with PLTP::ODP2 and Axig1::WUS Cassettes are Efficacious for the Rapid Selection of Streptomycin-Resistant Maize T0 Plantlets.

Immature embryos are harvested from Pioneer inbred PHR03 10-12 days after pollination. The immature embryos are placed on high-osmotic medium to induce plasmolysis. Meanwhile, three plasmids PHP91619 (containing PRO::UBI1ZM 5′ UTR::UBI1ZM INTRON1:FRT1:CTP::SPCN::SB-UBI TERM, SEQ ID NO: 3), PHP75799 (containing ZM-PLTP PRO::ZM-PLTP 5′ UTR::ZM-ODP2::OS-T28 TERM, SEQ ID NO: 4), and PHP76976 (containing ZM-AXIG1 PRO::ZM-WUS2::IN1-2 TERM, SEQ ID NO: 5) are individually precipitated onto 0.6 μM gold particles and introduced into the scutellar cells of the immature embryos. As a control, just PHP75799 (SEQ ID NO: 4) and PHP76976 (SEQ ID NO: 5) are used, with no SPCN-containing plasmid. After particle bombardment and culturing the zygotic immature embryos on the media series described in A above, with either 50 mg/l or 100 mg/l streptomycin, no streptomycin-resistant, green plantlets are recovered in the control treatments (PHP75799 (SEQ ID NO: 4) and PHP76976 (SEQ ID NO: 5)). However, in the treatment in which all three plasmids are introduced (PHP91619 (SEQ ID NO: 3), PHP75799 (SEQ ID NO: 4), and PHP76976 (SEQ ID NO: 5)), streptomycin-resistant, green T0 plantlets are produced at a frequency of between 10-30% relative to the number of bombarded immature embryos.

The maize-codon-optimized SPCN gene (SEQ ID NO: 11) is efficacious for conferring resistance to streptomycin in maize.

### Example 4: Rapid Recovery of Streptomycin Resistant Sorghum Plants

### A. Determining an Effective Streptomycin Concentration for Inhibition of Somatic Embryo Germination and Growth of Non-Transgenic Sorghum Plants.

TX430, a non-tannin sorghum variety, was used in this study. Greenhouse temperatures averaged 29° C. during the day and 20° C. at night with a 12 h day/night photoperiod and supplemental lighting is provided by a 3:1 ratio of metal halide (1,000 W) and high-pressure sodium (1,000 W) lamps. The components of the media used in this study are listed in Table 14. The baseline transformation protocol is described in detail as “treatment C” in Zhao et al. (Plant Mol. Biol. (2000) 44:789-798). Briefly, freshly harvested sorghum immature grains were sterilized with 50% bleach and 0.1% Tween-20 for 30 minutes under vacuum and then rinsed with sterile water three times. The embryos were subjected to the following five sequential steps: (1) Agrobacterium infection: embryos were incubated in an Agrobacterium suspension (OD=1.0 at 550 nm) with PHI-I medium for five minutes; (2) co-cultivation: embryos were cultured on PHI-T medium following infection for three days at 25° C. in the dark; (3) resting: embryos were cultured on PHI-T medium plus 100 mg/L carbenicillin for seven days at 28° C. in the dark; (4) selection: embryos were cultured on PHI-U medium (in which either 25, 50, 100 or 150 mg/L streptomycin replaces PPT as the selective agent in Table 14) for two weeks, followed by culture on PHI-V medium (in which either 25, 50, 100 or 150 mg/L streptomycin replaces PPT as the selective agent in Table 14) for the remainder of the selection process at 28° C. in the dark, using subculture intervals of two to three weeks; (5) regeneration: callus was cultured on PHI-X medium (in which either 25, 50, 100 or 150 mg/L streptomycin replaces PPT as the selective agent in Table 14) for two to three weeks in the dark to stimulate shoot development, followed by culture for one week under conditions of 16 hours light (40-120 μE/m²/s) and 8 hours dark at 25° C., and a final subculture on PHI-Z medium for two to three weeks under lights (16 h, 40-120 μE/m²/s) to stimulate root growth. Regenerated plantlets were transplanted into soil and grown in the greenhouse (Zhao et al. 2000). T0 plants were self-pollinated to produce T1 progeny for further analysis.

After transformation with Agrobacterium strain LBA4404 THY-containing PHP71539 (SEQ ID NO: 1) and PHP92307 (SEQ ID NO: 32) containing the T-DNA expression cassette RB+LOXP+PLTP:WUS:IN2-1 TERM+ZMHSP17.7:MO-CRE:PINII TERM+UBI1ZMPRO:NPTII:SB-UBI TERM+UBI1ZM PRO-FRT1 FRT1:CTP::SPCN::SB-UBI TERM), the immature embryos were cultured as described above.

Plantlets on 25 mg/L streptomycin displayed a mixture of green and bleached (white) leaves, on 50 mg/L all of the shoots were bleached. On 100 mg/L shoots were small and weak and all were bleached. No shoots germinated on 150 mg/L streptomycin. Based on this data, either 50 or 100 mg/L streptomycin was used for selection while expressing the maize-optimized SPCN gene in sorghum.

B. SPCN Expression Cassette Along with PLTP::ODP2 and Axig1::WUS Cassettes are Efficacious for the Rapid Selection of Streptomycin-Resistant Sorghum T0 Plantlets.

Immature embryos are harvested from sorghum variety TX430. The immature embryos are placed on high-osmotic medium to induce plasmolysis. Meanwhile, three plasmids PHP91619 (containing PRO::UBI1ZM 5′ UTR::UBI1ZM INTRON1:FRT1:CTP::SPCN::SB-UBI TERM, SEQ ID NO: 3), PHP75799 (containing ZM-PLTP PRO::ZM-PLTP 5′ UTR::ZM-ODP2::OS-T28 TERM, SEQ ID NO: 4), and PHP76976 (containing ZM-AXIG1 PRO::ZM-WUS2::IN1-2 TERM, SEQ ID NO: 5) are individually precipitated onto 0.6 μM gold particles and introduced into the scutellar cells of the immature embryos. As a control, just PHP75799 (SEQ ID NO: 4) and PHP76976 (SEQ ID NO: 5) are used, with no SPCN-containing plasmid. After particle bombardment and culturing the zygotic immature embryos on the media series described in A above, with either 50 mg/L or 100 mg/L streptomycin, no streptomycin-resistant, green plantlets are recovered in the control treatments (PHP75799 (SEQ ID NO: 4) and PHP76976 (SEQ ID NO: 5)). However, in the treatment in which all three plasmids are introduced (PHP91619 (SEQ ID NO: 3), PHP75799 (SEQ ID NO: 4), and PHP76976 (SEQ ID NO: 5)), streptomycin-resistant, green T0 plantlets are produced at a frequency of between 10-30% relative to the number of bombarded immature embryos.

The maize-codon-optimized SPCN gene (SEQ ID NO: 11) is efficacious for conferring resistance to streptomycin in sorghum.

### Example 5: Rapid Recovery of Streptomycin Resistant Wheat Plants

### A. Determining an Effective Streptomycin Concentration for Inhibition of Somatic Embryo Germination and Growth of Non-Transgenic Wheat Plants.

An aliquot of Agrobacterium strain LBA4404 containing the vector of interest is removed from storage at −80° C. and streaked onto solid LB medium containing a selective agent (kanamycin or spectinomycin, depending on which plasmids the bacterial strain contains). The Agrobacterium is cultured on the LB plate at 21° C. in the dark for two to three days, at which time a single colony is selected from the plate, streaked onto an 810D medium (5 g/l yeast extract, 10 g/l peptone, 5 g/l NaCl, adjust pH TO 6.8 with NaOH, 15 g/l bacto-agar, autoclave and cool to 60° C., then add the appropriate selective agent) and is then incubated at 28° C. in the dark overnight. The Agrobacterium culture is transferred from the plate using a sterile spatula and suspended in ˜5 mL wheat infection medium (WI 4) with 400 μM acetosyringone (AS). The optical density (600 nm) of the suspension is adjusted to about 0.1 to 0.7 using the same medium.

Four to five spikes containing immature seeds (with 1.4-2.3 mm embryos) are collected, and the immature embryos are isolated from the immature seeds. The wheat grains are surface sterilized for fifteen minutes in 20% (v/v) bleach (5.25% sodium hypochlorite) plus 1 drop of Tween 20, followed with two to three washes in sterile water. After transformation with Agrobacterium strain LBA4404 THY-containing PHP71539 (SEQ ID NO: 1) and PHP92307 (SEQ ID NO: 32) containing the T-DNA expression cassette RB+LOXP+PLTP:WUS:IN2-1 TERM+ZMHSP17.7:MO-CRE:PINII TERM+UBI1ZMPRO:NPTII:SB-UBI TERM+UBI1ZM PRO-FRT1 FRT1:CTP::SPCN::SB-UBI TERM), the immature embryos are co-cultured for one day at 21° C. on 562V medium, then transferred to 605B medium for eleven to fourteen days at 28° C. in the dark, transferred to 605B medium containing no streptomycin (control) or either 25, 50, 100 or 150 mg/L streptomycin for three weeks at 28° C. in the dark, and then transferred to 13329B medium (with or without the various concentrations of streptomycin described above) for somatic embryo maturation for two to three weeks at 28° C. in the dark. At this point, the shoots are transferred to light for two to three days and their vigor and leaf color is evaluated (data not shown).

Plantlets on 25 mg/L streptomycin display a mixture of green and bleached (white) leaves, on 50 mg/L all of the shoots are bleached. On media containing 100 mg/L streptomycin, the shoots are small and weak and all are bleached. No shoots germinate on 150 mg/L streptomycin. Based on this data, either 50 or 100 mg/L streptomycin is used for selection while expressing the maize-optimized SPCN gene in wheat.

B. SPCN Expression Cassette Along with PLTP::ODP2 and Axig1::WUS Cassettes are Efficacious for the Rapid Selection of Streptomycin-Resistant Wheat T0 Plantlets.

Immature embryos are harvested from Pioneer Spring wheat variety SPC0456D. The immature embryos are placed on high-osmotic medium to induce plasmolysis. Meanwhile, three plasmids PHP91619 (containing PRO::UBI1ZM 5′ UTR::UBI1ZM INTRON1:FRT1:CTP::SPCN::SB-UBI TERM, SEQ ID NO: 3), PHP75799 (containing ZM-PLTP PRO::ZM-PLTP 5′ UTR::ZM-ODP2::OS-T28 TERM expression cassette, SEQ ID NO: 4), and PHP76976 (containing ZM-AXIG1 PRO::ZM-WUS2::IN1-2 TERM expression cassette, SEQ ID NO: 5) are individually precipitated onto 0.6 μM gold particles and introduced into the scutellar cells of the immature embryos. As a control, just PHP75799 (SEQ ID NO: 4) and PHP76976 (SEQ ID NO: 5) are used, with no SPCN-containing plasmid. After particle bombardment and culturing the zygotic immature embryos on the media series described in A above, with either 50 mg/L or 100 mg/L streptomycin, no streptomycin-resistant, green plantlets are recovered in the control treatments (PHP75799 (SEQ ID NO: 4) and PHP76976 (SEQ ID NO: 5)). However, in the treatment in which all three plasmids are introduced (PHP91619 (SEQ ID NO: 3), PHP75799 (SEQ ID NO: 4), and PHP76976 (SEQ ID NO: 5)), streptomycin-resistant, green T0 plantlets are produced at a frequency of between 10-30% relative to the number of bombarded immature embryos.

The maize-codon-optimized SPCN gene (SEQ ID NO: 11) is efficacious for conferring resistance to streptomycin in wheat.

### Example 6: Rapid Recovery of Streptomycin Resistant Rice Plants

### A. Determining an Effective Streptomycin Concentration for Inhibition of Somatic Embryo Germination and Growth of Non-Transgenic Rice Plants.

To determine the effective amount of streptomycin to use in selection of transgenic rice plants Oryza sativa (v. indica IRV95) is subjected to experimental conditions that included Agrobacterium transformation but with a non-functional dicot cassette containing the SPCN gene. Immature embryos are isolated ten to twelve days after pollination. The immature embryos are split evenly between five treatments; a control treatment in which the embryos were cultured on 605J medium with no selection or 605J medium with either 25, 50, 100 or 150 mg/L streptomycin. After transformation with Agrobacterium strain LBA4404 THY- (disclosed in U.S. Pat. No. 8,334,429 and incorporated herein by reference in its entirety) containing PHP71539 (SEQ ID NO: 1) (disclosed in U.S. Patent Appln. No. US20190078106 and incorporated herein by reference in its entirety) and PHP92307 (SEQ ID NO: 32) containing the T-DNA expression cassette RB+LOXP+PLTP:WUS:IN2-1 TERM+ZMHSP17.7:MO-CRE:PINII TERM+UBI1ZMPRO:NPTII:SB-UBI TERM+UBI1ZM PRO-FRT1 FRT1:CTP::SPCN::SB-UBI TERM), the immature embryos are co-cultured for one day at 21° C. on 562V medium, moved to 605J medium for eleven to fourteen days at 28° C. in the dark (resting no selection), then transferred to 605J control medium or 605J medium containing the respective concentrations of streptomycin described above for three weeks at 28° C. in the dark, followed by transfer to 289Q medium (289Q control medium or 289Q medium with the various concentrations of streptomycin described above) for somatic embryo maturation for two to three weeks at 28° C. in the dark. Then the shoots are transferred to light for two to three days and their vigor and leaf color is evaluated (data not shown).

Rice plantlets on 25 mg/L streptomycin display a mixture of green and bleached (white) leaves, on 50 mg/L all of the shoots are bleached. On media containing 100 mg/L streptomycin the shoots are small and weak and all are bleached. No shoots germinate on 150 mg/L streptomycin. This data shows that streptomycin concentrations between 50 or 100 mg/L is used for selection of transgenic rice plants expressing the maize-optimized SPCN gene.

B. SPCN Expression Cassette Along with PLTP::ODP2 and Axig1::WUS Cassettes are Efficacious for the Rapid Selection of Streptomycin-Resistant Rice T0 Plantlets.

Immature embryos are harvested for rice indica variety IRV95 eleven to twelve days after pollination. The immature embryos are placed on high-osmotic medium to induce plasmolysis. Meanwhile, three plasmids PHP91619 (containing PRO::UBI1ZM 5′ UTR::UBI1ZM INTRON1:FRT1:CTP::SPCN::SB-UBI TERM, SEQ ID NO: 3), PHP75799 (containing ZM-PLTP PRO::ZM-PLTP 5′ UTR::ZM-ODP2::OS-T28 TERM, SEQ ID NO: 4), and PHP76976 (containing ZM-AXIG1 PRO::ZM-WUS2::IN1-2 TERM, SEQ ID NO: 5) are individually precipitated onto 0.6 μM gold particles and introduced into the scutellar cells of the immature embryos. As a control, just PHP75799 (SEQ ID NO: 4) and PHP76976 (SEQ ID NO: 5) are used, with no SPCN-containing plasmid. After particle bombardment and culturing the zygotic immature embryos on the media series described in A above, with either 50 mg/L or 100 mg/L1 streptomycin, no streptomycin-resistant, green plantlets are recovered in the control treatments (PHP75799 (SEQ ID NO: 4) and PHP76976 (SEQ ID NO: 5)). However, in the treatment in which all three plasmids are introduced (PHP91619 (SEQ ID NO: 3), PHP75799 (SEQ ID NO: 4), and PHP76976 (SEQ ID NO: 5)), streptomycin-resistant, green T0 plantlets are readily produced at a frequency between 10-30% relative to the number of bombarded immature embryos.

The maize-codon-optimized SPCN gene (SEQ ID NO: 11) is efficacious for conferring resistance to streptomycin in rice.

### Example 7: Rapid Recovery of Streptomycin Resistant Setaria Plants

### A. Determining an Effective Streptomycin Concentration for Inhibition of Somatic Embryo Germination and Growth of Non-Transgenic Setaria Plants.

Seed from Setaria viridis are surface sterilized in half-strength Clorox for fifteen minutes, are washed three times in sterile, distilled water, are blotted dry using sterile filter papers and then are placed on half-strength MS medium for germination. After fourteen days of growth, the leaves of the sterile Setaria seedlings are diced using a sterile #11 scalpel blade and the leaf segments aliquoted evenly between five treatments; a control treatment in which the embryos are cultured on 605J medium with no selection, or the same medium with either 25, 50, 100 or 150 mg/L streptomycin. After transformation with Agrobacterium strain LBA4404 THY-containing PHP71539 (SEQ ID NO: 1) and PHP1 (SEQ ID NO.: 33) containing the T-DNA expression cassette RB+LoxP-NOS:WUS:PINII+ZMUBI:ODP2:PINIFRAB17:MOCRE:PINII+ZMUBI-CTP-SPCN:SB-UBITERM+LB; the leaf segments are co-cultured for one day at 21° C. on 562V medium, and then undergo a series of transfers; first to 605B medium for eleven to fourteen days at 28° C. in the dark, next onto 605B medium containing the respective concentrations of streptomycin for three weeks at 28° C. in the dark, and then onto 13329B medium (with the various concentrations of streptomycin described above) for somatic embryo maturation for another two to three weeks at 28° C. in the dark. Then the shoots are transferred to light for two to three days and their vigor and leaf color is evaluated (data not shown).

Plantlets on media containing 25 mg/L streptomycin display a mixture of green and bleached (white) leaves, on media containing 50 mg/L all of the shoots are bleached. On media containing 100 mg/L shoots are small and weak and all are bleached. No shoots germinate on media containing 150 mg/L streptomycin. Based on this data, either 50 or 100 mg/L streptomycin is used for selection while expressing the maize-optimized SPCN gene in Setaria.

B. SPCN Expression Cassette Along with UBI:ODP2 and NOS:WUS Cassettes are Efficacious for the Rapid Selection of Streptomycin-Resistant Setaria T0 Plantlets.

Setaria seed are sterilized, germinated to produce fourteen-day old plantlets, and the leaf tissue is diced into 2-3 mm segments as described above. The leaf segments are then transformed using Agrobacterium strain LBA4404 THY-containing PHP71539 (SEQ ID NO: 1) (the VIR-containing helper plasmid) and a co-habitating plasmid PHP1 (SEQ ID NO: 33) with the following T-DNA; RB+LoxP-NOS:WUS:PINII+ZMUBI: ODP2:PINIFRAB 17:MOCRE:PINII+ZMUBI-CTP-SPCN: SB-UBITERM+LB (treatment with SPCN expression cassette). As a control treatment, leaf segments are transformed with Agrobacterium strain LBA4404 THY-containing PHP71539 (SEQ ID NO: 1) (the VIR-containing helper plasmid) and a co-habitating plasmid PHP2 (SEQ ID NO: 34) with the following T-DNA; RB+LoxP-NOS:WUS:PINII+ZMUBI:ODP2:PINIFRAB17:MOCRE:PINII+LB (control treatment with no SPCN expression cassette). After Agrobacterium-mediated transformation and culturing the leaf segments on the media series described in Section A above, with each medium after the resting medium containing either 50 mg/L or 100 mg/L streptomycin, no streptomycin-resistant, green plantlets are recovered in the control treatment. However, in the treatment containing the SPCN expression cassette streptomycin-resistant, green T0 plantlets are produced at a frequency of between 10-30% relative to the number of seedlings infected with Agrobacterium.

### Example 8: Rapid Recovery of Streptomycin Resistant Teff Plants

### A. Determining an Effective Streptomycin Concentration for Inhibition of Somatic Embryo Germination and Growth of Non-Transgenic Teff Plants.

Seeds from Eragrostis tef are surface sterilized in half-strength Clorox for fifteen minutes, are rinsed three times in sterile, distilled water, are blotted dry using sterile filter papers and then are placed on half-strength MS medium for germination. After fourteen days of growth, the leaves of the sterile teff seedlings are diced using a sterile #11 scalpel blade and the leaf segments are aliquoted evenly between five treatments; a control treatment in which the embryos are cultured on 605J medium with no selection, or the same 605J medium with either 25, 50, 100 or 150 mg/L streptomycin. After transformation with Agrobacterium strain LBA4404 THY-containing PHP71539 (SEQ ID NO: 1) and PHP1 (SEQ ID NO: 33) containing the T-DNA expression cassette RB+LoxP-NOS:WUS:PINII+ZMUBI: ODP2:PINIFRAB 17:MOCRE:PINII+ZMUBI-CTP-SPCN: SB-UBITERM+LB, the leaf segments are co-cultured for one day at 21° C. on 562V medium, and then undergo a series of additional transfers; onto 605B medium for eleven to fourteen days at 28° C. in the dark, next onto 605B medium with no selection or 605B medium with either 25, 50, 100 or 150 mg/L streptomycin for three weeks at 28° C. in the dark, and then onto 13329B medium (with and without the various concentrations of streptomycin described above) for somatic embryo maturation for two to three weeks at 28° C. in the dark. At this point, the shoots are transferred to light for two to three days and their vigor and leaf color is evaluated (data not shown).

Plantlets on 25 mg/L streptomycin display a mixture of green and bleached (white) leaves, on 50 mg/L all of the shoots are bleached. On 100 mg/L, all the shoots are small and weak and all are bleached. No shoots germinate on 150 mg/L streptomycin. Based on this data, either 50 mg/L1 or 100 mg/L streptomycin is used for selection while expressing the maize-optimized SPCN gene in teff.

B. SPCN Expression Cassette Along with UBI:ODP2 and NOS:WUS Cassettes are Efficacious for the Rapid Selection of Streptomycin-Resistant Eragrostis tef T0 Plantlets.

Teff seeds are sterilized, germinated to produce fourteen-day old plantlets, and the leaf tissue is diced into 2-3 mm segments as described above. The leaf segments are then transformed using Agrobacterium strain LBA4404 THY-containing PHP71539 (SEQ ID NO: 1) (the VIR-containing helper plasmid) and a co-habitating plasmid PHP1 (SEQ ID NO: 33) with the following T-DNA; RB+LoxP-NOS:WUS:PINII+ZMUBI: ODP2:PINIFRAB 17:MOCRE:PINII+ZMUBI-CTP-SPCN: SB-UBITERM+LB (treatment with SPCN expression cassette). As a control treatment, leaf segments are transformed with Agrobacterium strain LBA4404 THY-containing PHP71539 (SEQ ID NO: 1) (the VIR-containing helper plasmid) and a co-habitating plasmid PHP2 (SEQ ID NO: 34) with the following T-DNA; RB+LoxP-NOS:WUS:PINII+ZMUBI:ODP2:PINIFRAB17:MOCRE:PINII+LB (control treatment with no SPCN expression cassette). After Agrobacterium-mediated transformation and culturing the leaf segments on the media series described in Section A above, with each medium after the resting medium containing either 50 mg/L or 100 mg/L streptomycin, no streptomycin-resistant, green plantlets are recovered in the control treatment. However, in the treatment containing the SPCN expression cassette streptomycin-resistant, green T0 plantlets are produced at a frequency of between 10-30% relative to the number of seedlings infected with Agrobacterium.

### Example 9: Rapid Recovery of Streptomycin Resistant Sugarcane Plants

### A. Determining an Effective Streptomycin Concentration for Inhibition of Somatic Embryo Germination and Growth of Non-Transgenic Sugarcane Plants

Sterile plantlets of sugarcane are obtained in vitro through meristem proliferation, and are maintained in multiple shoot culture to produce starting leaf explants for transformation. Leaves from the sterile plantlets are diced using a sterile #11 scalpel blade and the leaf segments aliquoted evenly between five treatments: a control treatment in which the embryos are cultured on 605J medium with no selection, or the same medium with either 25, 50, 100 or 150 mg/L streptomycin. After transformation with Agrobacterium strain LBA4404 THY-containing PHP71539 (SEQ ID NO: 1) and PHP1 (SEQ ID NO: 33) containing the T-DNA expression cassette RB+LoxP-NOS:WUS:PINII+ZMUBI:ODP2:PINIFRAB17:MOCRE:PINII+ZMUBI-CTP-SPCN:SB-UBITERM+LBthe leaf segments are co-cultured for one day at 21° C. on 562V medium, moved to 605B medium for eleven to fourteen days at 28° C. in the dark (resting no selection), then transferred to 605B control medium or 605B medium containing the respective concentrations of streptomycin described above for three weeks at 28° C. in the dark, transferred to 13329B medium (with and without the various concentrations of streptomycin) for somatic embryo maturation for two to three weeks at 28° C. in the dark. Then the shoots are transferred to light for two to three days and their vigor and leaf color is evaluated (data not shown).

Plantlets on 25 mg/L streptomycin display a mixture of green and bleached (white) leaves, on 50 mg/L all of the shoots are bleached. On 100 mg/L, the shoots are small and weak and all are bleached. No shoots germinated on 150 mg/L streptomycin. Based on this data, either 50 mg/L or 100 mg/L streptomycin is used for selection while expressing the maize-optimized SPCN gene in sugarcane.

B. SPCN Expression Cassette Along with UBI:ODP2 and NOS:WUS Cassettes are Efficacious for the Rapid Selection of Streptomycin-Resistant Sugarcane T0 Plantlets

Sterile plantlets of sugarcane are obtained in vitro through meristem proliferation, and are maintained in multiple shoot culture to produce starting leaf explants for transformation. Leaves from the sterile plantlets are diced using a sterile #11 scalpel blade and the leaf segments are then transformed with Agrobacterium strain LBA4404 THY-containing PHP71539 (SEQ ID NO: 1) (the VIR-containing helper plasmid) and a co-habitating plasmid PHP1 (SEQ ID NO: 33) with the following T-DNA; RB+LoxP-NOS:WUS:PINII+ZMUBI: ODP2:PINIFRAB 17:MOCRE:PINII+ZMUBI-CTP-SPCN: SB-UBITERM+LB (treatment with SPCN expression cassette). As a control treatment, leaf segments are transformed with Agrobacterium strain LBA4404 THY-containing PHP71539 (SEQ ID NO: 1) (the VIR-containing helper plasmid) and a co-habitating plasmid PHP2 (SEQ ID NO: 34) with the following T-DNA; RB+LoxP-NOS:WUS:PINII+ZMUBI:ODP2:PINIFRAB17:MOCRE:PINII+LB (control treatment with no SPCN expression cassette). After Agrobacterium-mediated transformation and culturing the leaf segments on the media series described in Section A above, with each medium after the resting medium containing either 50 mg/L or 100 mg/L streptomycin, no streptomycin-resistant, green plantlets are recovered in the control treatment. However, in the treatment containing the SPCN expression cassette streptomycin-resistant, green T0 plantlets are produced at a frequency of between 10-30% relative to the number of seedlings infected with Agrobacterium.

### Example 10: Rapid Recovery of Streptomycin Resistant Plants

Similar experiments to those described above produce similar results in pearl millet, barley, oats, and flax. In addition, in species that are readily transformable without the use of morphogenic genes the SPCN expression cassette is used alone to recover transgenic events.

### Example 11: Improved Recovery of Site-Specific Recombination Events Using Streptomycin Selection in Maize

Immature ears are harvested from PH184C and 2.0 mm immature embryos are extracted from the kernels on the day of the particle bombardment treatment. The embryos are placed on high osmotic medium (13224B medium) for three hours prior to particle bombardment. Immature embryos are bombarded with an equimolar ratio of four plasmids containing the following expression cassettes: 1) FRT1:CTP::SPCN::PINII TERM:FRT87 (PHP3, SEQ ID NO: 35); 2) UBI1ZM PRO::FLPm::PINII TERM (PHP5096, SEQ ID NO: 18); 3) ZM-PLTP PRO::ZM-PLTP 5′ UTR::ZM-ODP2::OS-T28 TERM (PHP75799, SEQ ID NO: 4); and 4) ZM-AXIG1 PRO::ZM-WUS2::IN2-1 TERM (PHP76976, SEQ ID NO: 5). After particle bombardment, the immature embryos remain on the high-osmotic medium overnight, and are then transferred to resting medium (13266K) for eight days. After the resting period, the embryos are transferred to maturation medium (2890 medium with 100 mg/L streptomycin) for twenty-one days, and then moved onto rooting medium (272X medium with 50 mg/L streptomycin) for fourteen to seventeen days (until the roots are large enough for transplanting into soil). At the plantlet stage, leaf tissue is sampled for PCR analysis to confirm that the genes within the flanking FRT1 and FRT87 sites of the original target locus are no longer present, that the new genes within the donor cassette have recombined into the target locus correctly, and precise RMCE (Recombinase-Mediated Cassette Exchange) events are identified. This reduces the entire site-specific integration (SSI) cycle, from transformation to having precise RMCE-derived plants in the greenhouse, down to from forty-three to fifty days, depending on how much time is required to produce adequate roots.

Alternately, an Agrobacterium-mediated SSI method is used. The T-DNA delivered contains SPCN, WUS2, ODP2 and DsRED expression cassettes within the flanking FRT1 and FRT87 recombination sites (PHP4, SEQ ID NO: 36) with the following T-DNA (RB+UBI PRO:UBI1ZM INTRON::MO-FLP::PINII TERM+CaMV35S TERM+FRT1:CTP::SPCN::PINII TERM:FRT87+UBI PRO::UBI1ZM INTRON::DsRED+NOS PRO::ZM-WUS2::PINII TERM+UBI PRO:UBI1ZM INTRON::ZM-ODP2::PINII TERM+LB). The T-DNA is delivered via Agrobacterium-mediated transformation into target lines with FRT1-FRT87 landing sites as described in U.S. Patent Appln. No. 20170240911, incorporated herein by reference in its entirety. Precise RMCE events are identified using a multiplex PCR assay as described in U.S. Patent Appln. No. 20170240911, incorporated herein by reference in its entirety. The use of SPCN in the promoter trap, along with the NOS PRO::WUS2+UBI PRO::ODP2 expression cassettes for Agrobacterium SSI reduces the SSI process by several weeks (at least three to four weeks), compared to the previous transformation method for generating SSI events.

### Example 12: Improved Recovery of Site-Specific Recombination Soy Events with SPCN Gene and Spectinomycin Selection

Soybean site specific integration (SSI) is done using a microbial delivery system. In soybean, there are two microbial transformation systems available for SSI transformation: the Ochrobactrum embryonic axis (EA) system disclosed in U.S. Patent Appln. No. 20180216123, incorporated herein by reference in its entirety and the Agrobacterium Immature Cotyledon (IC) system disclosed in U.S. Provisional Patent Appln. No. 62/610,540, filed Dec. 27, 2017, incorporated herein by reference in its entirety.

In the Agrobacterium Immature Cotyledon (IC) system immature soybean seeds are surface sterilized in a 50 mL screw cap tube containing 50 mL of a 10% bleach, 0.02% Tween-20 solution, with slight agitation for fifteen minutes and are then rinsed ten times with a total of 500 mL of sterile distilled water. Immature cotyledons are aseptically excised by cutting the embryo axis off the cotyledons and then pushing the cotyledons out of the seed coat onto sterile 7.5 cm filter paper moistened with sterile distilled water in a deep petri dish (25×100 mm). Twenty to Twenty-five isolated immature cotyledons are transferred into a sterile glass tube (16×100 mm) containing 400 μL of an Agrobacterium inoculum. Sonication (one second) is performed in a sonic water bath (VWR 50T). After sonication, the immature cotyledons are left in the inoculum for fifteen minutes at room temperature for infection. After fifteen minutes of infection, immature cotyledons from two glass tubes are poured onto double layered sterile filter papers (total 800 μl/double layered filter) in a deep petri dish and then the petri dishes are wrapped with two layers of Parafilm for co-cultivation for four days at 21° C. in a Percival brand incubator at a light intensity of 3-5 μE/m²/s.

After four days of co-cultivation, immature cotyledons are washed off the filter paper with S30 medium supplemented with 300 μg/mL timentin antibiotic and are rinsed three times to remove residual Agrobacterium. The immature cotyledons are then transferred to a 250 mL sterile glass flask (40-50 immature cotyledons/flask) containing 40-50 mL S30 medium supplemented with 300 mg/L timentin antibiotic to kill the Agrobacterium without selection, and are cultured at 25-26° C. with an 18-hour photoperiod at 35-60 μE/m²/s light intensity for seven days on rotary shaker at 100 rpm for the recovery period.

Following the recovery period, a selection agent is used for the selection of stable transformants. The recovery medium is replaced with 40-50 mL S30 medium supplemented with the selection agent for the selection of transformed cells. The selection medium is replaced bi-weekly and cultured at 25-26° C. with 18-hour photoperiod at 35-60 μE/m²/s light intensity on a rotary shaker at 100 rpm. After four to eight weeks on selection medium, transformed tissue becomes visible as green tissue against a background of bleached, less healthy tissue.

Putative transformed green callus is isolated under a microscope and plated onto petri plates with sterile filter paper overlaying M7 agar medium supplemented with 300 mg/L timentin for embryo maturation. The petri plates are sealed with Micropore™ surgical tape (3M Health Care, St. Paul, Minn., USA) and incubated at 26° C. with an 18-hour photoperiod at 35-60 μE/m²/s light intensity. After three to four weeks of maturation on M7 medium, mature somatic embryos are placed in sterile, petri dishes and either sealed with Micropore™ surgical tape or placed unsealed in a plastic box for four to seven days at room temperature for somatic embryo desiccation. After four to seven days, desiccated embryos are plated onto M8 medium supplemented with the selection agent and are allowed to germinate at 26° C. with an 18-hour photoperiod at 35-60 μE/m²/s light intensity. After four to six weeks on M8 germination medium, plantlets are transferred to four inch pots containing moistened Berger BM2 soil (Berger Peat Moss, Saint-Modeste, Canada) and kept enclosed in clear plastic tray boxes until acclimatized in a culture room with a 16-hour photoperiod at 90-150 μE/m²/s and 26° C. day/24° C. night temperatures. After acclimation, hardened plantlets are potted in 2 gallon pots containing moistened Berger MB1 (Berger Peat Moss, Saint-Modeste, Canada) and grown in a greenhouse to seed-bearing maturity.

In both systems (Ochrobactrum embryonic axis (EA) system and Agrobacterium Immature Cotyledon (IC) system, SPCN selection attenuates selection pressure and allows time for recombination to occur and thus produces site-specific integration (SSI) plants at a higher frequency than other selectable marker genes such as the ALSand hygromycin plant selectable marker genes.

Ochrobactrum EA SSI, was performed using a target line generated in the soybean genotype 93B86. The target line was generated by Agrobacterium-mediated immature cotyledon transformation using plasmid PHP49452 (SEQ ID NO.: 37) containing the expression cassette RB+GM-SAMS PRO-GM-SAMS UTR-GM SAMS INTRON1-GM-SAMS UTR2-FRT1:CAMV35S PRO:HYGNOS TERM+GM-UBQ PRO-GM-UBQ 5UTR:ZS-YELLOW:NOS TERM-FRT87+LB. Homozygous EAs derived from this target line were retransformed with three different binary plasmids, PHP92521 (SEQ ID NO: 38) containing the expression cassette RB+AT-UBIQ10 PRO:FLP:UBQ3TERM+FRT1-CTP-SPCN:UBQ10 TERM++GM-MYH11:DS-RED:PINII-FRT87+LB, PHP92985 (SEQ ID NO: 39) containing the expression cassette RB+GM-EF1A2PRO:FLP:UBQ10 TERM+FRT1-CTP-SPCN:UBQ3 TERM++GM-MYH11PRO:DS-RED:PINII-FRT87+LB, and PHP93448 (SEQ ID NO: 40) containing the SPCN expression cassette flanked by the and FRT87 sites (RB+GMEF1A2 PRO:FLP:UBQ3 TERM+FRT1-CTP-SPCN:UBQ10 TERM+FMVENH+PCSV EHN+MMV ENH+GM-MTH1:DS-RED:PINII-FRT87+LB). Following Ochro-EA re-transformation of the target line, SSI events were recovered on a media supplemented with spectinomycin to select events that were transformed with the SPCN selectable marker gene replacing the hygromycin gene in the target line. The transformation protocol included of the following steps: i) dry seeds were sterilized with chlorine gas in a closed chamber in a chemical hood; ii) the sterilized seeds were imbibed for 6-8 hours on 5 g/L sucrose and 6 g/L agar medium followed by overnight soaking in water; iii) embryonic axes explants were isolated either manually or with the assistance of mechanical tools; iv) the embryonic axes were infected with Ochrobactrum containing SSI vectors with the SPCN selectable marker gene; v) explants were placed on co-cultivation medium for three to four days; vi) explants were transferred to selection medium (spectinomycin 25 mg/L) immediately after co-cultivation or allowed to recover on medium without selection for one to two days; vii) after continued biweekly subculture on shoot elongation medium with selection, elongated shoots were harvested at around six weeks for rooting, and viii) T0 plants with roots were sent to the greenhouse. The plants selected on spectinomycin were recovered and were analysed by qPCR. The SSI events were identified using a number of assays designed to detect the target and donor DNA. Specifically, SSI events were identified using qPCRs for detecting the target DNA and the donor DNA following selection on media supplemented with spectinomycin. Events positive for FRT1 and FRT87 junctions with one copy each of SPCN, ZS-YELLOW, and target line were considered SSI events. The results are shown in Table 6). Specifically, the transformation data detailing the number of EAs infected, total shoots harvested, T0 plants generated, the transformation frequency and the SSI frequency using Ochro-EA transformation of soybean with SPCN as the selectable marker gene are shown in Table 6.

TABLE 6

| | | | ZS- | Target | | FRT1: | PSE: | DS- | | | | |
|---|---|---|---|---|---|---|---|---|---|---|---|---|
| Plant | SSI | SPCN | YELLOW | line | PSE_3_1 | CTP | FRT 87 | RED | PSD_15_1 | PSC_26_1 | FLPM | NPTII |
| ID | (+/−) | Copy # | Copy # | (+/−) | Copy # | (+/−) | (+/−) | Copy # | (+/−) | (+/−) | (+/−) | (+/−)I |
| 1 | + | 1 | 1 | + | 1 | + | + | 1 | − | − | − | − |
| 2 | − | 1 | 1 | + | 0 | + | − | 0 | − | − | − | − |
| 3 | + | 1 | 1 | + | 1 | + | + | 1 | − | − | − | − |
| 4 | − | 1 | 0 | + | 0 | + | − | 0 | + | − | − | − |
| 5 | + | 1 | 1 | + | 1 | + | + | 1 | − | − | − | − |
| 6 | + | 1 | 1 | + | 1 | + | + | 1 | − | + | − | − |
| 7 | + | 2 | 1 | + | 1 | + | + | 1 | − | − | − | − |

The transformation data detailing the number of EAs infected, total shoots harvested, T0 plants generated, the transformation frequency and the SSI frequency using Ochro-EA transformation of soybean with SPCN as the selectable marker gene are shown in Table 7.

TABLE 7

| | # of EAs | # of shoots | # ofT0 plants | SSI |
|---|---|---|---|---|
| Vector ID | infected | harvested | generated | Frequency |
| PHP92521 | 662 | 10 (1.5%) | 3 (0.45%) | 2 (0.3%) |
| PHP92985 | 720 | 3 (0.41%) | 2 (0.27%) | 2 (0.27%) |
| PHP93448 | 716 | 6 (0.83%) | 2 (0.27%) | 1 (0.13%) |

When Agrobacterium-mediated transformation was used to deliver T-DNA into soybean immature cotyledons, SSI events were recovered when SPCN was used in the promoter-trap for selection (US Patent Application No. 20140157453, incorporated herein by reference in its entirety). For Agrobacterium-mediated transformation of immature cotyledons, the following steps were included: i) seed pods were opened and immature seeds of 2-4 mm were removed and surface sterilized with 5% Clorox bleach for ten minutes, then the seeds were rinsed three times with sterile, distilled water; ii) the cotyledons were excised and pre-cultured in S30 medium in flasks for two days; iii) the immature cotyledons were then infected by adding a solution of Agrobacterium (OD 0.5 at 500 nm) in M5 infection medium (U.S. Pat. No. 8,962,328 incorporated herein by reference in its entirety); iv) the explants were co-cultured with the Agrobacterium for days days at 21° C.; v) the explants were washed with liquid medium containing 300 mg/L Timentin and transferred to liquid medium with 300 mg/L Timentin to recover for one week; vi) the explants were transferred to selection medium with 300 mg/L Timentin and 25 mg/L spectinomycin for four to five weeks with medium changes every two weeks; vii) healthy, growing events were transferred to maturation medium for tree weeks; viii) the healthy embryos were dried down for one week and germinated on germination medium; and ix) T0 plantlets were potted to soil in the greenhouse.

### Example 13. SPCN Gene and Spectinomycin Selection for Canola Transformation

Seeds of Brassica napus were surface sterilized in a 50% Clorox solution and germinated on solid medium containing MS basal salts and vitamins. The seedlings were grown at 28° C. in the light for ten to fourteen days, and the hypocotyls were dissected away from the cotyledons. The hypocotyl explants were transferred into 100×25 mm petri plates containing 10 mls of 20A medium with 200 mM acetosyringone and then sliced into sections 3-5 mm long. After slicing, 40 μl of Agrobacterium solution (at an Optical Density of 0.50 at 550 nm) containing PHP88871 (SEQ ID NO: 6) was added to the plates, and the petri plates containing the hypocotyl/Agrobacterium mixture were placed on a shaker platform and lightly agitated for ten minutes. After ten minutes of gentle agitation, the plates were moved into dim light and 21° C. for three days of co-cultivation.

After co-cultivation, the hypocotyl explants were removed from the Agrobacterium solution, and lightly blotted onto sterile filter paper before placing onto 70A selection media (containing 10 mg/l spectinomycin) and moved to the light room (26° C. and bright light). Explants remained on 70A selection media for two weeks prior to transfer to a second round of 70A selection (alternatively, explants were moved to 70B medium with 20 mg/l spectinomycin for the second round of selection). After two rounds of selection the explants were transferred to 70C shoot elongation media for two to three weeks and placed back into the light room. Shoots were then transferred onto 90A rooting media before being transferred to soil in the greenhouse. The production of transgenic shoots exhibiting spectinomycin resistance occurred at a frequency of approximately 60-70% relative to the number of starting hypocotyl explants (datat not shown).

### Example 14. SPCN Gene and Spectinomycin Selection for Sunflower Transformation

A. Agrobacterium strain LBA4404 THY- was used for sunflower (Helianthus annuus variety F1503LG) transformation. Mature dry sunflower seeds were sterilized in a bleach solution (10-15% v/v in water with one drop of Tween detergent) for fifteen (15) minutes and then rinsed three (3) times in sterile water. Seeds were imbibed overnight. The embryos were removed from the softened hulls. Once the embryos were isolated, incisions were made at the base of the cotyledons to facilitate embryo isolation thereby exposing the leaf primordia sheathing the apical meristem. The radical tip was left attached to the embryo. After isolation, embryo axes (EAs) were transferred to petri plates for infection. The Agrobacterium strain containing plasmid PHP92349 (SEQ ID NO: 41) with a T-DNA containing expression cassettes for the SPCN (spectinomycin resistance) and DS-RED2 (fluorescence) genes, specifically containing RB+LOXP+GM-QBU PRO::CTP:SPCN::UBQ14 TERM+GM-EF1A2 PRO::DS-RED2::UBQ3 TERM+LOXP+LB was used for transformation. The Agrobacterium strain was suspended in 20A media and the concentration of the bacterial suspensions were adjusted to 0.5 OD550. The EAs were then placed under vacuum with gentle agitation for twenty (20) minutes. The EAs were removed from the Agrobacterium and inserted radicle-end down into 272AC medium (standard MS salts and vitamin levels (Murashige and Skoog, 1962, Physiol. Plant 15:473-497), 0.1 g/l myo-inositol, 50 mg/l thymidine, 100 uM acetosyringone, 0.1 mg/l BAP, 40 g/l sucrose, 6 g/l Bacto Agar, pH 5.6), leaving the apical dome above the 272AC medium, and placed under dim light at 21° C. for three days of co-cultivation. After co-cultivation, the EAs were transferred to 272AB spectinomycin selection media (standard MS salts and vitamin levels (Murashige and Skoog, 1962, Physiol. Plant 15:473-497), 0.1 g/l myo-inositol, 10 mg/l meropenum, 0.1 mg/l meta-Topolin (mT), 30 mg/l spectinomycin dihydrochloride, 0.1 ug/l, 40 g/l sucrose, 6 g/l Bacto Agar, pH 5.6) under full light at 28° C.

The EAs were allowed to grow, with periodic trimming of bleached leaves. Within approximately three weeks after exposure to spectinomycin, green sectors or whole green leaves were observed. This green tissue also expressed DS-RED, confirming that the T-DNA from PHP92349 had been integrated. For rooting, transgenic events were transferred to a Bio-Dome Sponge rooting material (Park Seed Co., 3507 Cokesbury Road, Hodges, S.C.). After transfer to the Bio-Dome Sponge rooting material, roots formed within one to three weeks and the plants were potted and transferred to the greenhouse or growth-chambers. Transgenic plantlets were recovered at a rate of 1% (relative to the number of starting explants infected with Agrobacterium). These results (data not shown) demonstrated that the SPCN gene was a useable selectable marker for rapid detection of transformed events and for improving transformation efficiency in sunflower.

B. Seeds of Helianthus annuus were surface sterilized for 15 minutes in a 50% Clorox solution, rinsed three times with sterile distilled water, and then soaked overnight in water to soften the seed coat and were germinated on solid medium containing MS basal salts and vitamins. Embryos were removed from the seed coat by squeezing the large end of the seed and pulling the embryo out. If the seed coat was still firm, the coat was scored with the tip of a #11 scalpel blade to help release the embryo. Once the embryo was isolated, one cotyledon was scored near the base (just above where the cotyledon meets the radical). The scalpel blade was positioned between the cotyledons and twisted to remove one of the cotyledons. At this point, the plumule was exposed against the other cotyledon. The tip of the #11 scalpel blade was placed along the inside base of the remaining cotyledon and cut across the plumule to remove both the cotyledon and tip of the plumule, leaving the meristem and radicle as the target explants for transformation. The target explants were placed in a petri plate. Once all explants had been transferred to the petri dish, an Agrobacterium suspension (OD 0.50 at 550 nm) containing PHP81356 (SEQ ID NO: 7) was added to the plant tissue, adding enough Agrobacterium suspension to cover the explants. Agrobacterium was prepared in 20A media supplemented with 200 mM acetosyringone. The suspension containing the sunflower explants and the Agrobacterium was placed on a shaker platform and gently agitated under standard house vacuum for 10-15 minutes.

Explants were then removed from the Agrobacterium suspension and placed onto solid medium 710I (containing no 2,4-D) or 272V medium (with 200 mM acetosyringone, 0.1 mg/L BAP), with the radical in contact with the medium and the apical meristem end up. The co-cultivation plates were then placed under dim light at 21° C. for overnight co-culture. After co-culture explants were moved to 272M (272X medium plus 1 mg/L meropenem) supplemented with 40 mg/L spectinomycin and then placed into the light room (26° C., 16 hours light).

After two weeks, the first leaves emerging from the plantlets were typically bleached, and were trimmed back close to the meristem and the plantlets were placed back into the light room. When vigorous, green leaves were observed, care was taken to trim away as much of the bleached leaves as possible to give preference to the emerging green growth. Plantlets with emerging green leaves were moved to fresh media with reduced spectinomycin (20 mg/L). If lateral roots were observed growing from the radicle, no further stimulation was required for root growth. If spontaneous root growth was not observed, the plantlets were moved to 272 medium (4.3 g MS basal salt mixture, 0.1 g myo-inositol, 0.5 mg nicotinic acid, 1 mg thiamine HCl, 0.5 mg pyridoxine.HCl, 2 mg glycine, 40 g sucrose, 1.5 g Gelrite, pH 5.6.) with 0.1 mg/L NAA (naphthaleneacetic acid). Once a green shoot was established the new plantlet was removed from the spectinomycin selection. Transgenic plantlets were recovered at a rate of 1% (relative to the number of starting explants infected with Agrobacterium).

### Example 15. SPCN Gene and Spectinomycin Selection for Cassava Transformation

Leaf-petiole explants (whole immature leaves with 1-1.5 mm of the petiole attached) are excised from six to eight-week old plantlets of cassava cultivar TME7 and are placed on MS basal medium (2% sucrose, 0.8% Noble agar, 1 μM 2,4-D and 1 μM meta-Topolin (mT) (see Chauhan and Taylor, 2018 Plant Cell Tiss Organ Cult 132:219-224)). For cassava, expression cassettes containing either the wild-type Streptomyces spectabilis SPCN gene (SEQ ID NO: 9) or the soy-codon-optimized version of the gene (SEQ ID NO: 13) can be used (both of which encode the same protein, SEQ ID NOs: 10 and 14). After two weeks of culture, green nodular structures form which can be used as the target explant for Agrobacterium-mediated transformation using PHP88871 (SEQ ID NO: 6) (OD 0.50 at 550 nm). The Agrobacterium suspension is added to the plant tissue to a volume sufficient to cover the explants. Agrobacterium is prepared in 20A media supplemented with 200 mM acetosyringone. The suspension containing the cassava explants and the Agrobacterium is placed on a shaker platform and gently agitated under standard house vacuum for ten to fifteen minutes. Explants are then removed from the Agrobacterium suspension and placed onto solid medium containing 1 μM meta-Topolin (mT) with 200 mM acetosyringone. The co-cultivation plates are then placed under dim light at 21° C. for overnight co-culture. After co-culture explants are moved onto solid medium containing 1 μM meta-Topolin (mT) plus 40 mg/L spectinomycin and then placed into the light room (26° C., 16-hours light). After two weeks, the first leaves emerging from the plantlets are typically bleached, and are trimmed back close to the meristem and the plantlets placed back into light room. When vigorous, green leaves are observed, care is taken to trim away as much of the bleached leaves as possible to give preference to the emerging green growth. Plantlets with emerging green leaves are moved to fresh media with reduced spectinomycin (20 mg/L). If lateral roots are observed growing from the radicle, no further stimulation is required for root growth. If spontaneous root growth is not observed, the plantlets are moved to 272 medium with 0.1 mg/L NAA (naphthaleneacetic acid). Once a green shoot is established the new plantlet is removed from the spectinomycin. Using this method, transgenic plantlets are recovered at a rate of 1-3% (relative to the number of starting explants infected with Agrobacterium).

### Example 16. SPCN Gene and Spectinomycin Selection for Soybean Transformation

Mature dry seed from soybean lines were surface-sterilized for sixteen hours using chlorine gas, produced by mixing 3.5 mL of 12 N HCl with 100 mL of commercial bleach (5.25% sodium hypochloride), as described by Di et al., ((1996) Plant Cell Rep 15:746-750). Disinfected seeds were soaked in sterile distilled water at room temperature for sixteen hours (100 seeds in a 25×100 mm petri dish) and imbibed on semi-solid medium containing 5 g/L sucrose and 6 g/L agar at room temperature in the dark. After overnight incubation, the seeds were soaked in distilled water for an additional three to four hours at room temperature in the dark. Intact embryonic axes (EA) were isolated from cotyledons. Ochrobactrum-mediated EA transformation was carried out as described herein and disclosed in U.S. Patent Appln. No. 20180216123, incorporated herein by reference in its entirety. The compositions of various cultivation media used for soybean EA transformation and plant regeneration are summarized in Table 17.

A volume of 10 mL of Ochrobactrum haywardense HI suspension (OD 0.50 at 600 nm) in infection medium containing 300 mM acetosyringone was added to the EA. The embryonic axes were co-cultivated with the Ochrobactrum haywardense HI suspension containing PHP82311 (SEQ ID NO: 26), PHP82312 (SEQ ID NO: 27), PHP82313 (SEQ ID NO: 28) or PHP82314 (SEQ ID NO: 8). The plates were sealed with parafilm (“Parafilm M” VWR Cat #52858), then sonicated (Sonicator-VWR model 50T) for thirty seconds. After sonication, about 90-500 embryonic axes were transferred to a single layer of autoclaved sterile filter paper (VWR #415/Catalog #28320-020). The plates were sealed with Micropore tape (Catalog #1530-0, 3M, St. Paul, Minn.)) and incubated under dim light (1-2 μE/m²/s), cool white fluorescent lamps for sixteen hours at 21° C. for three days. After co-cultivation, the base of each EA was embedded in shoot induction (SI) medium containing Spectinomycin 25 mg/L and 500 mg/L Cefotaxime. Shoot induction was carried out in a Percival Biological Incubator or growth room at 26° C. with a photoperiod of sixteen hours and a light intensity of 60-100 μE/m²/s. After three to six weeks in selection medium, transformed spectinomycin-resistant shoots were produced from infected meristems of EA. The transformed shoots were cut and transferred to rooting medium for further shoot and root elongations.

Spectinomycin resistant healthy shoots were produced from the shoot apical meristem of embryonic axes of Soybean variety 93Y21 transformed with Ochrobactrum haywardense HI suspension containing PHP82311 (SEQ ID NO: 26), PHP82312 (SEQ ID NO: 27), PHP82313 (SEQ ID NO: 28) or PHP82314 (SEQ ID NO: 8). Most of the spectinomycin resistant shoots expressed red fluorescent protein (RFP). Untransformed apical meristem of embryonic axes, on the other hand, were bleached in selection medium containing spectinomycin at concentrations of 25 mg/L or higher. Soybean embryonic axes transformed with Ochrobactrum haywardense HI containing SPCN expression cassettes PHP82313 (SEQ ID NO: 28) or PHP82314 (SEQ ID NO: 8) showed an even expression pattern of RFP in the spectinomycin resistant shoots, while PHP82311 (SEQ ID NO: 26) and PHP82312 (SEQ ID NO: 27) showed an uneven expression pattern of RFP in the spectinomycin resistant shoots which indicated chimeric expression (data not shown). Transformed shoots of 0.5 to 2 cm in height were produced within five to six weeks of transformation. Transformation efficiencies (relative to the number of embryonic axes transformed with Ochrobactrum haywardense HI containing vectors) ranged from 10% to 17.4% as shown in Table 8 below.

TABLE 8

| | Total number of | Total number of spectinomycin |
|---|---|---|
| | embryonic axes | resistant shoots showing RFP |
| Construct | transformed | expression (% TE) |
| PHP82311 (SEQ ID | 158 | 16 (10.1%) |
| NO: 26) | | |
| PHP82312 (SEQ ID | 161 | 28 (17.4%) |
| NO: 27) | | |
| PHP82313 (SEQ ID | 166 | 25 (15.1%) |
| NO: 28) | | |
| PHP82314 (SEQ ID | 160 | 16 (10.0%) |
| NO: 8) | | |

Embryonic axes of soybean varieties 93Y21, P29T50, P33T60, DM118, 98C11 and 98C21 transformed with Ochrobactrum haywardense HI containing PHP82314 (SEQ ID NO: 8), as described in this Example 17, produced spectinomycin resistant healthy shoots from the shoot apical meristem of the embryonic axes. All varities produced transformed T0 events two and a half to three months after transformation. Transformation efficiencies ranged from 0.5 to 21.3% at T0 event production as shown in Table 9 below.

TABLE 9

| | Number of T0/Number of EAs | |
|---|---|---|
| Variety | transformed | TE % |
| 93Y21 | 101/744 | 13.6 |
| P29T50 | 36/169 | 21.3 |
| P33T60 | 22/711 | 3.1 |
| DM118 | 9/498 | 1.8 |
| 98C11 | 2/440 | 0.5 |
| 98C21 | 94/644 | 15.7 |

### Example 17. Tobacco Leaf Disc Transformation Using APH and Spectinomycin Genes as Selectable Markers

Tobacco leaf disk transformation was performed as described by Gallois and Marinho (Methods Mol Biol 49:39-48, 1995). Tobacco plants (Nicotiana tabacum cv Petite Havana SR1, Catalog #NT-02-20-01, Lehle Seeds, Round Rock, Tex.) were aseptically cultured in a sterile polypropylene container (Catalog #0701, International Container Corp, Severn, Md.) containing half-strength Murashige and Skoog (MS) medium with 1.5% sucrose and 0.3% Gelrite under sixteen hours light (50-70 μE/m²/s cool white fluorescent lamps) at 26° C. Log phase Agrobacterium tumefaciens strain AGL1 cultures without a binary vector (Negative Control) and with a binary vector PHP81354 (SEQ ID NO: 30), PHP81355 (SEQ ID NO: 29), PHP81356 (SEQ ID NO: 7) or PHP81359 (SEQ ID NO: 31) were centrifuged at 3,000×G for ten minutes and the respective cell pellets of AGL1 were then diluted to an OD 0.50 at 600 nm with liquid co-cultivation medium composed of MS medium (pH 5.2) with 1 mg/L N6-benzyladenine (BA), 1% glucose and 200 μM acetosyringone.

Sterile tobacco leaves were excised from plants and soaked in 20 mL of AGL1 culture, containing a Negative Control (without a binary vector) or with a binary vector, namely, PHP81354 (SEQ ID NO: 30), PHP81355 (SEQ ID NO: 29), PHP81356 (SEQ ID NO: 7) or PHP81359 (SEQ ID NO: 31), in liquid co-cultivation medium in 100×25 mm Petri dishes for five minutes. Leaves were then cut into approximately 3×3 mm segments and the leaf pieces were then fully submerged in 20 mL of the AGL1 culture, containing a Negative Control (without a binary vector) or with a binary vector PHP81354 (SEQ ID NO: 30), PHP81355 (SEQ ID NO: 29), PHP81356 (SEQ ID NO: 7) or PHP81359 (SEQ ID NO: 31) for five minutes. Leaf segments were blotted onto autoclaved filter paper, then incubated on solid co-cultivation medium composed of MS medium (pH 5.2) with 1 mg/L BA, 1% glucose, 200 acetosyringone and Phytoagar (Catalog #A175, PhytoTechnology Laboratories, Shawnee Mission, Kans.) under sixteen hours light (80-110 μE/m²/s, cool white fluorescent lamps) at 24° C.

After three days of co-cultivation, twenty leaf segments/plate were transferred to shoot induction medium composed of MS solid medium (pH 5.7) with 1 mg/L BA, 3% sucrose, 0.3% Gelrite, 250 μg/mL Timentin containing 0, 250, 500, or 1,000 μg/mL spectinomycin. Tobacco leaf disks transformed with empty AGL1 (Negative Control) were completely bleached on shoot induction medium containing 250, 500, and 1,000 μg/mL spectinomycin. Conversely, tobacco leaf disks transformed with AGL1 containing a binary vector PHP81354 (SEQ ID NO: 30), PHP81355 (SEQ ID NO: 29), PHP81356 (SEQ ID NO: 7) or PHP81359 (SEQ ID NO: 31) produced dark green, healthy, spectinomycin-resistant shoots (>hundreds/plate) on shoot induction medium containing 250, 500, and 1,000 μg/mL spectinomycin within two to four weeks after transformation.

### Example 18. Soybean Hairy Root Transformation Using the Spectinomycin Gene (SPCN or APH) as a Selectable Marker

Soybean hairy root transformation was done as described by Cho et al. (Planta 210:195-204, 2000). Soybean 93Y21 seeds were surface-sterilized by soaking in 20% (v/v) commercial bleach, 5.25% (v/v) sodium hypochlorite, with Tween 20 (0.1%) for twenty minutes and then rinsed five times in sterile distilled water. Sterilized seeds were germinated on sucrose (0.5%) and agar (1.2%) medium under sixteen hours light (45 μE/m²/s cool white fluorescent lamps) at 25° C. Agrobacterium rhizogenes strain K599 cultures without a binary vector (Negative Control) or with a binary vector PHP81354 (SEQ ID NO: 30, (SAMS PRO::APH)), PHP81355 (SEQ ID NO: 29, (SAMS PRO::SPCN)), PHP81356 (SEQ ID NO: 7, (UBI PRO::SPCN)) or PHP81359 (SEQ ID NO: 31, (UBI PRO::APH)) were grown to log phase. The Agrobacterium cells were centrifuged at 3,000×G for ten minutes, and then the cell pellet was diluted to an OD 0.50 at 600 nm by adding liquid co-cultivation medium to the bacteria. The co-cultivation medium was composed of MS (PhytoTechnology M404) liquid medium (pH 5.2) with 30 g glucose and 300 μM acetosyringone.

Cotyledons from four to five day old-soybean seedlings were harvested and infected by first wounding the abaxial surface of the leaf with a scalpel, then adding the wounded leaf tissue to 20 ml of K599 culture, containing a Negative Control (no binary vector) or a binary vector PHP81354 (SEQ ID NO: 30, (SAMS PRO::APH)), PHP81355 (SEQ ID NO: 29, (SAMS PRO::SPCN)), PHP81356 (SEQ ID NO: 7, (UBI PRO::SPCN)) or PHP81359 (SEQ ID NO: 31, (UBI PRO::APH)) in liquid co-cultivation medium in 100×25 mm Petri dishes. The cotyledons were then fully submerged in the 20 ml of K599 culture, containing the Negative Control (no binary vector) or the binary vector PHP81354 (SEQ ID NO: 30, (SAMS PRO::APH)), PHP81355 (SEQ ID NO: 29, (SAMS PRO::SPCN)), PHP81356 (SEQ ID NO: 7, (UBI PRO::SPCN)) or PHP81359 (SEQ ID NO: 31, (UBI PRO::APH)) for twenty-five minutes.

Cotyledons were cultured abaxial side up on double-layer filter paper immersed in 4 ml sterile distilled water. Three days after inoculation, cotyledons were transferred (abaxial side up) to selection medium, MS (Murashige and Skoog 1962) basal nutrient salts, B5 (Gamborg et al. 1968) vitamins and 3% sucrose (pH 5.7), solidified with 3 g/L Gelrite (Greif Bros. Corp., East Coast Division, Spotswood, N.J., USA) with 250 mg/L Timentin and 0, 50, 100, 250 or 500 spectinomycin respectively. Cotyledons inoculated with K599 lacking the binary vector (Negative Control) produced only a small amount of callus at the infection sites on the cotyledons in spectinomycin containing medium. On the other hand, soybean cotyledons transformed with K599 containing any of the binary vectors PHP81354 (SEQ ID NO: 30, (SAMS PRO::APH)), PHP81355 (SEQ ID NO: 29, (SAMS PRO::SPCN)), PHP81356 (SEQ ID NO: 7, (UBI PRO::SPCN)) or PHP81359 (SEQ ID NO: 31, (UBI PRO::APH)) produced highly branched hairy roots which developed from each wound site on the cotyledons in the presence of spectinomycin fourteen to sixteen days after selection.

### Example 19. Cowpea Embryonic Axis Transformation Using the SPCN Gene as a Selectable Marker

Cowpea was used for Agrobacterium-mediated transformation of embryonic axis. Transgenic events were obtained by transforming embryonic axes using Agrobacterium strain LBA4404 containing PHP71539 (SEQ ID NO: 1) and PHP86170 (SEQ ID NO: 25). PHP86170 (SEQ ID NO: 25) carries SPCN and DS-RED expression cassettes. Cowpea seeds were sterilized with 20% Clorox bleach for fifteen minutes, rinsed three times in sterile, distilled water, and then imbibed in water overnight. Fifty embryonic axes were isolated manually and were suspended in Infection Medium (Table 18) with Agrobacterium (OD 0.5 at 550 nm) containing PHP71539 (SEQ ID NO: 1) or PHP86170 (SEQ ID NO: 25) for two to three hours. Explants were then transferred to Co-cultivation Medium (Table 18) for three days in an incubator at 21° C. in the dark. After co-cultivation, explants were transferred to shoot regeneration medium (SIM) (Table 18) containing 25 mg/L spectinomycin. Explants that developed shoots after four weeks were transferred again to SIM plus spectinomycin for an additional three to four weeks. Transgenic shoots with DsRed expression were then moved onto shoot elongation medium plus spectinomycin (Table 18). Well-developed shoots were transferred to rooting medium with spectinomycin (Table 18) for three to five weeks, and healthy plants were sent to the greenhouse. The timeframe from Agrobacterium infection to sending T0 plantlets to the greenhouse was rapid (˜four months), and the transformation frequency was 4%.

### Example 20: SPCN Selection Improves Recovery of Cas9/CRISPR-Mediated Genomic Modified Events

CAS9-mediated targeted homologous recombination is used to select for events in which the PRO::UBI1ZM 5′ UTR::UBI1ZM INTRON1:FRT1:CTP::SPCN::SB-UBI TERM (PHP91619, SEQ ID NO: 3) expression cassette is introduced into the maize liguleless1 locus. The experimental design used here reproduces that described in Svitashev et al. (2015, Plant Physiol. 169:931-945) utilizing the same plasmids, T-DNA components, expression cassettes, Particle Gun-mediated transformation, culturing media, and maize germplasm (Hi-II) as those described in the above article with one exception. Svitachev used a UBI1ZM PRO::moPAT::pinII TERM expression cassette in between the two flanking homology arms (sequences from the ligulelessl locus that were upstream (1099-bp) or downstream (1035-bp) of the LIG-CR3-guided cut site in the genome), while the PRO::UBI1ZM 5′ UTR::UBI1ZM INTRON1:FRT1:CTP::SPCN::SB-UBI TERM (PHP91619, SEQ ID NO: 3) sequence replaces the moPAT expression cassette between the two liguless1 homology arms in this example. All other experimental parameters are identical in order to compare the efficiency of SPCN selection for recovery of homology directed repair (HDR) events relative to the efficiency reported for moPAT selection by Svitashev et al.

Using the particle gun, the plasmid containing the donor cassette (Homology Arm+PRO::UBI1ZM 5′ UTR::UBI1ZM INTRON1:FRT1:CTP::SPCN::SB-UBI TERM (PHP91619, SEQ ID NO: 3)+Homology Arm) is co-bombarded with 1) a plasmid containing CAS9 and LIG-CR3 gRNA expression cassettes, 2) ZM-AXIG1 PRO::ZM-WUS2::IN2-1 TERM (SEQ ID NO: 5), and 3) ZM-PLTP PRO::ZM-PLTP 5′ UTR::ZM-ODP2::OS-T28 TERM (SEQ ID NO: 4). After transformation, the immature embryos culture, selection and regeneration are performed as described in Gordon-Kamm et al. (2002, PNAS 99:11975-11980) except that the herbicide bialaphos is replaced with 50 mg/L streptomycin. After progressing through callus selection, somatic embryo maturation and plant regeneration on streptomycin as the selective agent, healthy green plantlets are recovered. The frequency of recovering streptomycin resistant plants is similar to the results observed in Svitashev et al. with the moPAT gene and bialaphos selection, and molecular analysis confirms a similar number of precisely-integrated donor sequences into the liguleless1 locus via homologous recombination.

### Example 21. SPCN Gene and Spectinomycin Selection for Cotton Transformation

Seeds of cotton lines are surface sterilized in a 50% Clorox solution or for sixteen hours using chlorine gas, produced by mixing 3.5 mL of 12 N HCl with 100 mL of commercial bleach (5.25% sodium hypochloride), as described by Di et al., (1996) Plant Cell Rep 15:746-750. Disinfected seeds are soaked in sterile distilled water at room temperature for sixteen hours (100 seeds in a 25×100 mm petri dish) and imbibed on semi-solid medium containing 5 g/L sucrose and 6 g/L agar at room temperature in the dark. After overnight incubation, the seeds are soaked in distilled water for an additional three to four hours at room temperature in the dark. Intact embryonic axes (EA) are isolated from cotyledons

Ochrobactrum or Agrobacterium-mediated EA or hypocotyl transformation is carried out as described below. The bacterial strains used for transformation included Ochrobactrum haywardense HI strain containing a Ri plasmid PHP81184 (SEQ ID NO: 42) or different strains of Agrobacterium including AGL1, LBA4404 with or without PHP70298 (SEQ ID NO: 43), PHP71539 (SEQ ID NO: 1) and PHP79761 (SEQ ID NO: 44).

A volume of 10 mL of Ochrobactrum haywardense HI suspension (OD₆₀₀ 0.5 to 1.0) or Agrobacterium suspension (OD₆₀₀ 0.5 to 1.0) in infection medium containing 200 mM acetosyringone is added to the EA. The embryonic axes are co-cultivated with the O. haywardense HI or Agrobacterium suspension containing PHP81356 (SEQ ID NO: 7). After co-cultivation, the base of each EA is embedded in shoot induction (SI) medium containing Spectinomycin 25-50 mg/L and 500 mg/L Cefotaxime. Shoot induction is carried out in a Percival Biological Incubator or growth room at 26° C. with a photoperiod of sixteen hours and a light intensity of 60-100 μl E/m²/s. After three to six weeks in selection medium, transformed spectinomycin-resistant shoots are produced from infected meristems of EA. The transformed shoots are cut and transferred to rooting medium for further shoot and root elongations. Using these methods, transgenic plantlets are recovered at a rate of 1-3% (relative to the number of starting explants infected with Ochrobactrum or Agrobacterium).

For hypocotyl transformation, surface sterilized cotton seeds are grown at 28° C. in the light for ten to fourteen days, and the hypocotyls are dissected away from the cotyledons. The hypocotyl is sliced into sections 3-5 mm long. Segments are transferred into 100×25 mm petri plates containing 12 ml of 20A medium with 100 mM acetosyringone. After slicing, 20 μl of Agrobacterium solution or Ochrobacturum solution (OD600 0.5 to 1.0) containing PHP81356 (SEQ ID NO: 7) is added to the plates, and the petri plates containing the hypocotyl/Agrobacterium or Ochrobacturum mixture are placed on a shaker platform and lightly agitated for ten minutes. After ten minutes of gentle agitation, the plates are moved into dim light and 21° C. for two to four days of co-cultivation. After co-cultivation, the hypocotyl explants are removed from the bacterial solution, and lightly blotted onto sterile filter paper before placing onto selection media (containing 10-20 mg/L spectinomycin) and moved to the light room (26° C. and bright light). After few rounds of selection, the explants are transferred to a shoot elongation media for two to three weeks and placed back into the light room. Shoots are then transferred onto a rooting media before being transferred to soil in the greenhouse. The green transgenic shoots are collected and transferred to greenhouse. Using these methods, transgenic plantlets are recovered at a rate of 1-3% (relative to the number of starting explants infected with Ochrobactrum or Agrobacterium).

### Example 22: Media

A wide range of tissue or explant types can be used in the current methods, including suspension cultures, immature cotyledons, mature cotyledons, split seed, embryonic axes, hypocotyls, embryos, and epicotyls. See Tables 10-18 for a description of the media formations for transformation, selection and regeneration referenced in the Examples.

TABLE 10

| | Units | | | | | | | | | |
|---|---|---|---|---|---|---|---|---|---|---|
| Medium components | per liter | 12R | 810K | 700A | 710I | 605B | 605J | 605T | 562V | 289Q |
| MS BASAL | G | | | 4.3 | 4.3 | 4.3 | 4.3 | 4.3 | | 4.3 |
| SALT | | | | | | | | | | |
| MIXTURE | | | | | | | | | | |
| N6 BASAL | G | | | | | | | | 4.0 | |
| SALTS | | | | | | | | | | |
| N6 | ml | | | | | 60.0 | 60.0 | 60.0 | | |
| MACRONUTRIENTS | | | | | | | | | | |
| 10X | | | | | | | | | | |
| POTASSIUM NITRATE | G | | | | | 1.7 | 1.7 | 1.7 | | |
| B5H | ml | | | | | 0.6 | 0.6 | 0.6 | | |
| MINOR | | | | | | | | | | |
| SALTS | | | | | | | | | | |
| 1000X | | | | | | | | | | |
| NaFe EDTA | ml | | | | | 6.0 | 6.0 | 6.0 | | |
| FORB5H | | | | | | | | | | |
| 100X | | | | | | | | | | |
| ERIKSSON'S | ml | | | | | 0.4 | 0.4 | 0.4 | 1.0 | |
| VITAMINS | | | | | | | | | | |
| 1000X | | | | | | | | | | |
| S&H | ml | | | | | 6.0 | 6.0 | 6.0 | | |
| VITAMIN | | | | | | | | | | |
| STOCK | | | | | | | | | | |
| 100X | | | | | | | | | | |
| THIAMINE•HCL | mg | | | 10.0 | 10.0 | 0.5 | 0.5 | 0.5 | 0.5 | |
| L-PROLINE | G | | | | 0.7 | 2.0 | 2.0 | 2.0 | 0.69 | 0.7 |
| CASEIN | G | | | | | 0.3 | 0.3 | 0.3 | | |
| HYDROLYSATE | | | | | | | | | | |
| (ACID) | | | | | | | | | | |
| SUCROSE | G | | | 68.5 | 20.0 | 20.0 | 20.0 | 20.0 | 30.0 | 60.0 |
| GLUCOSE | G | 5.0 | | 36.0 | 10.0 | 0.6 | 0.6 | 0.6 | | |
| MALTOSE | G | | | | | | | | | |
| 2,4-D | mg | | | 1.5 | 2.0 | 1.6 | 0.8 | 0.8 | 2.0 | |
| AGAR | G | 15.0 | | | 8.0 | 6.0 | 6.0 | 6.0 | 8.0 | 8.0 |
| BACTO- | G | | 15.0 | | | | | | | |
| AGAR | | | | | | | | | | |
| PHYTAGEL | G | | | | | | | | | |
| DICAMBA | g | | | | | 1.2 | 1.2 | 1.2 | | |
| SILVER | mg | | | | | 1.7 | 3.4 | 3.4 | 0.85 | |
| NITRATE | | | | | | | | | | |
| AGRIBIO | mg | | | | | 100.0 | 100.0 | | | |
| Carbenicillin | | | | | | | | | | |
| Timentin | mg | | | | | | | 150.0 | | 150.0 |
| Cefotaxime | mg | | | | | | | 100.0 | | 100.0 |
| Meropenem | mg | | | | | 50.0 | | | | |
| MYO- | g | | | 0.1 | 0.1 | | | | | 0.1 |
| INOSITOL | | | | | | | | | | |
| NICOTINIC | mg | | | 0.5 | 0.5 | | | | | |
| ACID | | | | | | | | | | |
| PYRIDOXINE•HCL | mg | | | 0.5 | 0.5 | | | | | |
| VITAMIN | g | | | 1.0 | | | | | | |
| ASSAY | | | | | | | | | | |
| CASAMINO | | | | | | | | | | |
| ACIDS | | | | | | | | | | |
| MES | g | | | | 0.5 | | | | | |
| BUFFER | | | | | | | | | | |
| ACETOSYRINGONE | uM | | | | 100.0 | | | | 100.0 | |
| ASCORBIC | mg | | | | 10.0 | | | | | |
| ACID | | | | | | | | | | |
| 10 MG/ML | | | | | | | | | | |
| (7S) | | | | | | | | | | |
| MS | ml | | | | | | | | | 5.0 |
| VITAMIN | | | | | | | | | | |
| STOCK | | | | | | | | | | |
| SOL. | | | | | | | | | | |
| ZEATIN | mg | | | | | | | | | 0.5 |
| CUPRIC | mg | | | | | | | | | 1.3 |
| SULFATE | | | | | | | | | | |
| IAA | ml | | | | | | | | | 2.0 |
| 0.5 MG/ML | | | | | | | | | | |
| (28A) | | | | | | | | | | |
| ABA 0.1 mm | ml | | | | | | | | | 1.0 |
| THIDIAZURON | mg | | | | | | | | | 0.1 |
| AGRIBIO | mg | | | | | | | | | 100.0 |
| Carbenicillin | | | | | | | | | | |
| PPT(GLUFOSINATE- | mg | | | | | | | | | |
| NH4) | | | | | | | | | | |
| BAP | mg | | | | | | | | | 1.0 |
| YEAST | g | | 5.0 | | | | | | | |
| EXTRACT | | | | | | | | | | |
| (BD Difco) | | | | | | | | | | |
| PEPTONE | g | | 10.0 | | | | | | | |
| SODIUM | g | | 5.0 | | | | | | | |
| CHLORIDE | | | | | | | | | | |
| SPECTINOMYCIN | mg | 50.0 | 50.0 | | | | | | | |
| FERROUS | ml | 2.0 | | | | | | | | |
| SULFATE•7H20 | | | | | | | | | | |
| AB BUFFER | ml | 50.0 | | | | | | | | |
| 20X (12D) | | | | | | | | | | |
| AB SALTS | ml | 50.0 | | | | | | | | |
| 20X (12E) | | | | | | | | | | |
| THYMIDINE | mg | 50.0 | 50.0 | 50.0 | | | | | 50.0 | |
| GENTAMYCIN | mg | 50.0 | 50.0 | | | | | | | |
| Benomyl | mg | | | | | | | | | |
| pH | | | 6.8 | 5.2 | 5.8 | 5.8 | 5.8 | 5.8 | 5.8 | 5.6 |

TABLE 11

| | Units | | | | | | | |
|---|---|---|---|---|---|---|---|---|
| Medium components | per liter | 289R | 13158H | 13224B | 13266K | 272X | 272V | 13158 |
| MS BASAL SALT | g | 4.3 | 4.3 | | 4.3 | 4.3 | 4.3 | 4.3 |
| MIXTURE | | | | | | | | |
| N6 | ml | | | 4.0 | 60.0 | | | |
| MACRONUTRIENTS | | | | | | | | |
| 10X | | | | | | | | |
| POTASSIUM | g | | | | 1.7 | | | |
| NITRATE | | | | | | | | |
| B5H MINOR SALTS | ml | | | | 0.6 | | | |
| 1000X | | | | | | | | |
| NaFe EDTA FOR B5H | ml | | | | 6.0 | | | |
| 100X | | | | | | | | |
| ERIKSSON'S | ml | | | 1.0 | 0.4 | | | |
| VITAMINS 1000X | | | | | | | | |
| S&H VITAMIN | ml | | | | 6.0 | | | |
| STOCK 100X | | | | | | | | |
| THIAMINE•HCL | mg | | | 0.5 | 0.5 | | | |
| L-PROLINE | g | 0.7 | 0.7 | 2.9 | 2.0 | | | |
| CASEIN | g | | | | 0.3 | | | |
| HYDROLYSATE | | | | | | | | |
| (ACID) | | | | | | | | |
| SUCROSE | g | 60.0 | 60.0 | 190.0 | 20.0 | 40.0 | 40.0 | 40.0 |
| GLUCOSE | g | | | | 0.6 | | | |
| MALTOSE | g | | | | | | | |
| 2,4-D | mg | | | | 1.6 | | | |
| AGAR | g | | 8.0 | 6.4 | 6.0 | 6.0 | 6.0 | 6.0 |
| PHYTAGEL | g | | | | | | | |
| DICAMBA | g | | | | 1.2 | | | |
| SILVER NITRATE | mg | | | 8.5 | 1.7 | | | |
| AGRIBIO Carbenicillin | mg | | | | 2.0 | | | |
| Timentin | mg | 150.0 | 150.0 | | | | | |
| Cefotaxime | mg | 100.0 | 100.0 | 25 | 25 | | | |
| MYO-INOSITOL | g | 0.1 | 0.1 | | | 0.1 | 0.1 | 0.1 |
| NICOTINIC ACID | mg | | | | | | | |
| PYRIDOXINE•HCL | mg | | | | | | | |
| VITAMIN ASSAY | g | | | | | | | |
| CASAMINO ACIDS | | | | | | | | |
| MES BUFFER | g | | | | | | | |
| ACETOSYRINGONE | uM | | | | | | | |
| ASCORBIC ACID | mg | | | | | | | |
| 10 MG/ML (7S) | | | | | | | | |
| MS VITAMIN STOCK | ml | 5.0 | 5.0 | | | 5.0 | 5.0 | 5.0 |
| SOL. | | | | | | | | |
| ZEATIN | mg | 0.5 | 0.5 | | | | | |
| CUPRIC SULFATE | mg | 1.3 | 1.3 | | | | | |
| IAA 0.5 MG/ML (28A) | ml | 2.0 | 2.0 | | | | | |
| ABA 0.1 mm | ml | 1.0 | 1.0 | | | | | |
| THIDIAZURON | mg | 0.1 | 0.1 | | | | | |
| AGRIBIO Carbenicillin | mg | | | | | | | |
| PPT(GLUFOSINATE- | mg | | | | | | | |
| NH4) | | | | | | | | |
| BAP | mg | | | | | | | |
| YEAST EXTRACT | g | | | | | | | |
| (BD Difco) | | | | | | | | |
| PEPTONE | g | | | | | | | |
| SODIUM CHLORIDE | g | | | | | | | |
| SPECTINOMYCIN | mg | | | | | | | |
| FERROUS | ml | | | | | | | |
| SULFATE•7H20 | | | | | | | | |
| AB BUFFER 20X | ml | | | | | | | |
| (12D) | | | | | | | | |
| AB SALTS 20X (12E) | ml | | | | | | | |
| Benomyl | mg | | | | | | | 100.0 |
| pH | | | | | | 0.5 | 5.6 | |

TABLE 12

| | Units | | | | | |
|---|---|---|---|---|---|---|
| Medium components | per liter | 20A | 70A | 70B | 70C | 90A |
| MS BASAL SALT | g | 4.3 | 4.3 | 4.3 | 4.3 | 4.3 |
| MIXTURE | | | | | | |
| THIAMINE•HCL | mg | 0.12 | 0.12 | 0.12 | 0.12 | 0.12 |
| SUCROSE | g | | 20 | 20 | 20 | |
| PVP40 | g | | 0.5 | 0.5 | 0.5 | |
| TC AGAR | g | | 5 | 5 | 5 | 5 |
| SILVER NITRATE | mg | | 2.0 | 2.0 | 2.0 | |
| AGRIBIO Carbenicillin | g | | 0.5 | 0.5 | 0.5 | |
| Adenine Hemisulfate Salt | mg | | 40 | 40 | 40 | |
| MYO-INOSITOL | g | 0.1 | 0.1 | 0.1 | 0.1 | |
| NICOTINIC ACID | mg | 0.57 | 0.57 | 0.57 | 0.57 | |
| PYRIDOXINE•HCL | mg | 0.57 | 0.57 | 0.57 | 0.57 | |
| Glycine | mg | 2.3 | 2.3 | 2.3 | 2.3 | |
| MES BUFFER | g | 0.5 | 0.5 | 0.5 | 0.5 | |
| ACETOSYRINGONE | uM | 200 | | | | |
| NAA | mg | 0.1 | 0.1 | 0.1 | 0.1 | |
| BAP | mg | 1.0 | 1.0 | 1.0 | 1.0 | |
| IBA | mg | | | | | 0.5 |
| Gibberellic Acid | ug | 10 | 10 | 10 | 10 | |
| SPECTINOMYCIN | mg | | 5 | 10 | 10 | |
| pH | | | 5.7 | 5.7 | 5.7 | 5.7 |

The compositions of various media used in soybean transformation, tissue culture and regeneration are outlined in Table 13. In this table, medium M1 is used for initiation of suspension cultures, if this is the starting material for transformation. Media M2 and M3 represent typical co-cultivation media useful for Agrobacterium transformation of the entire range of explants listed above. Medium M4 is useful for selection (with the appropriate selective agent), M5 is used for somatic embryo maturation, and medium M6 is used for germination to produce T0 plantlets. Additional media compositions for Agrobacterium-mediated transformation of dicot plants are disclosed in U.S. Pat. No. 8,962,328 incorporated herein by reference in its entirety.

TABLE 13

| Medium components | M1 | | M2 | | M3 | | M4 | | M5 | | M6 | | S30 | | M7 | | M8 | |
|---|---|---|---|---|---|---|---|---|---|---|---|---|---|---|---|---|---|---|
| MS salt with B5 | 4.44 | g/L | | | | | 4.4 | g/L | | | | | | | 4.44 | g/L | | |
| vitamins | | | | | | | | | | | | | | | | | | |
| (PhytoTech | | | | | | | | | | | | | | | | | | |
| M404) | | | | | | | | | | | | | | | | | | |
| Gamborg B-5 | | | | | | | | | | | 3.21 | g/L | | | | | 3.21 | g/L |
| basal medium | | | | | | | | | | | | | | | | | | |
| (PhytoTech | | | | | | | | | | | | | | | | | | |
| G398) | | | | | | | | | | | | | | | | | | |
| Modified MS | | | 2.68 | g/L | 2.68 | g/L | | | 2.68 | g/L | | | 2.68 | g/L | | | | |
| salt (PhytoTech | | | | | | | | | | | | | | | | | | |
| M571) | | | | | | | | | | | | | | | | | | |
| B5 vitamins | | | 1 | ml | 1 | ml | 1 | ml | 1 | ml | | | 1 | ml | | | | |
| (1000X) | | | | | | | | | | | | | | | | | | |
| (PhytoTech | | | | | | | | | | | | | | | | | | |
| G249) | | | | | | | | | | | | | | | | | | |
| 2,4-D stock 10 | 4 | ml | 1 | ml | 1 | ml | 4 | ml | 1 | ml | | | 1 | ml | | | | |
| mg/ml | | | | | | | | | | | | | | | | | | |
| KNO₃ | | | 1.64 | g/L | 1.64 | g/L | | | 1.64 | g/L | | | 0.93 | g/L | | | | |
| (NH₄)₂SO₄ | | | 0.463 | g/L | 0.463 | g/L | | | 0.463 | g/L | | | 0.463 | g/L | | | | |
| Asparagine | | | 1 | g/L | 1 | g/L | | | 1 | g/L | | | 1 | g/L | | | | |
| Glutamine | | | | | | | | | | | | | 4.48 | g/L | | | | |
| L-Methionine | | | | | | | | | | | | | 0.149 | g/L | | | | |
| Sucrose | | | 68.5 | g/L | 85.6 | g/L | | | 68.5 | g/L | 20 | g/L | 10 | g/L | | | 10 | g/L |
| Glucose | 31.5 | g/L | 36 | g/L | 49.6 | g/L | 31.5 | g/l | 36 | g/L | | | | | | | | |
| Maltose | | | | | | | | | | | | | | | 60 | g/L | | |
| MgCl₂•6H₂O | | | | | | | | | | | | | | | 0.75 | g/L | | |
| Activated | | | | | | | | | | | | | | | 5 | g/L | | |
| charcoal | | | | | | | | | | | | | | | | | | |
| (PhytoTech | | | | | | | | | | | | | | | | | | |
| C325) | | | | | | | | | | | | | | | | | | |
| Casein | | | 1 | g/L | 1 | g/L | | | 1 | g/L | | | | | | | | |
| hydrolysate | | | | | | | | | | | | | | | | | | |
| (PhytoTech | | | | | | | | | | | | | | | | | | |
| C184) | | | | | | | | | | | | | | | | | | |
| pH | 7.0 | | 5.4 | | 5.4 | | 7.0 | | 5.4 | | 5.7 | | 5.8 | | 5.7 | | 5.7 | |
| Acetosyringone | | | 300 | μM | 300 | μM | | | 200 | μM | | | | | | | | |
| DTT | | | | | | | | | 1 | mM | | | | | | | | |
| TC agar | 4 | g/L | | | | | | | | | 5 | g/L | | | | | 5 | g/L |
| Gelrite (Plant | | | | | | | 2 | g/l | | | | | | | 2 | g/L | | |
| Media Cat# | | | | | | | | | | | | | | | | | | |
| 714246) | | | | | | | | | | | | | | | | | | |

After 1-5 days of co-culture, the tissue is cultured on M3 medium with no selection for one week (recovery period), and then moved onto selection. For selection, an antibiotic or herbicide is added to M3 medium for the selection of stable transformants. To begin counter-selection against Agrobacterium, 300 mg/l Timentin® (sterile ticarcillin disodium mixed with clavulanate potassium, PlantMedia, Dublin, Ohio, USA) is also added, and both the selective agent and Timentin® are maintained in the medium throughout selection (up to a total 8 weeks). The selective media is replaced weekly. After 6-8 weeks on selective medium, transformed tissue becomes visible as green tissue against a background of bleached (or necrotic), less healthy tissue. These pieces of tissue are cultured for an additional 4-8 weeks.

Green and healthy somatic embryos are then transferred to M5 media containing 100 mg/L Timentin®. After a total of 4 weeks of maturation on M5 media, mature somatic embryos are placed in a sterile, empty Petri dish, sealed with Micropore™ tape (3M Health Care, St. Paul, Minn., USA) or placed in a plastic box (with no fiber tape) for 4-7 days at room temperature.

Desiccated embryos are planted in M6 media where they are left to germinate at 26° C. with an 18-hour photoperiod at 60-100 μl E/m²/s light intensity. After 4-6 weeks in germination media, the plantlets are transferred to moistened Jiffy-7 peat pellets (Jiffy Products Ltd, Shippagan, Canada), and kept enclosed in clear plastic tray boxes until acclimatized in a Percival incubator under the following conditions, a 16-hour photoperiod at 60-100 μE/m²/s, 26° C./24° C. day/night temperatures. Finally, hardened plantlets are potted in 2 gallon pots containing moistened SunGro 702 and grown to maturity, bearing seed, in a greenhouse.

Standard protocols for particle bombardment as disclosed by Finer and McMullen, 1991, In Vitro Cell Dev. Biol. —Plant 27:175-182, Agrobacterium-mediated transformation as disclosed by Jia et al., 2015, Int J. Mol. Sci. 16:18552-18543 and in US Patent Application No. 20170121722, incorporated herein by reference in its entirety, or Ochrobactrum-mediated transformation as disclosed in U.S. Patent Appln. No. 20180216123, incorporated herein by reference in its entirety, can be used with the methods of the disclosure. Standard protocols for plastid transromation as disclosed by Zora Svab, Peter Hajdukiewicz, and Pal Maliga (1990) Stable transformation of plastids in higher plants, Proc. Natl. Acad. Sci. 87:8526-8530) and in U.S. Pat. No. 5,877,402, incorporated herein by reference in their entireties.

TABLE 14
Media For Sorghum Transformation
Medium Composition

PHI-I: 4.3 g/l MS salts (Phytotechnology Laboratories, Shawnee Mission, KS, catalog
number M524), 0.5 mg/l nicotinic acid, 0.5 mg/l pyridoxine HCl, 1 mg/l thiamine HCl,
0.1 g/l myo-inositol, 1 g/l casamino acids (Becton Dickinson and Company, BD
Diagnostic Systems, Sparks, MD, catalog number 223050), 1.5 mg/l 2,4-
dichlorophenoxyacetic acid (2,4-D), 68.5 g/l sucrose, 36 g/l glucose, pH 5.2; with
100 μM acetosyringone added before using.
PHI-T: PHI-I with 20 g/l sucrose, 10 g/l glucose, 2 mg/l 2,4-D, no casamino acids,
0.5 g/l MES buffer, 0.7 g/l L-proline, 10 mg/l ascorbic acid, 100 μM acetosyringone,
8 g/l agar, pH 5.8.
PHI-U: PHI-T with 1.5 mg/l 2,4-D 100 mg/l carbenicillin, 30 g/l sucrose, no glucose
and acetosyringone; 5 mg/l PPT, pH 5.8.
PHI-UM: PHI-U with 12.5 g/l mannose and 5 g/l maltose, no sucrose, no PPT, pH 5.8
PHI-V: PHI-U with 10 mg/l PPT
DBC3: 4.3 g/l MS salts, 0.25 g/l myo-inositol, 1.0 g/l casein hydrolysate, 1.0 mg/l
thiamine HCL, 1.0 mg/l 2,4-D, 30 g/l maltose, 0.69 g/l L-proline, 1.22 mg/l cupric
sulfate, 0.5 mg/l BAP (6-benzylaminopurine), 3.5 g/l phytagel, pH 5.8
PHI-X: 4.3 g/l MS salts, 0.1 g/l myo-inositol, 5.0 ml MS vitamins stock^{b}, 0.5 mg/l
zeatin, 700 mg/l L-proline, 60 g/l sucrose, 1 mg/l indole-3-acetic acid, 0.1 μM abscisic
acid, 0.1 mg/l thidiazuron, 100 mg/l carbenicillin, 5 mg/l PPT, 8 g/l agar, pH 5.6.
PHI-XM: PHI-X with no PPT; added 1.25 mg/l cupric sulfate, pH 5.6.
PHI-Z: 2.15 g/l MS salts, 0.05 g/l myo-inositol, 2.5 ml MS vitamins stock^{a}, 20 g/l
sucrose, 3 g/l phytagel, pH 5.6
^{a}MS vitamins stock: 0.1 g/l nicotinic acid, 0.1 g/l pyridoxine HCl, 0.02 g/l thiamine HCl, 0.4 g/l glycine.

TABLE 15
Composition of wheat liquid infection medium WI 4
WI 4

| | | |
|---|---|---|
| DI water | 1000 | mL |
| MS salt + Vitamins(M519) | 4.43 | g |
| Maltose | 30 | g |
| Glucose | 10 | g |
| MES | 1.95 | g |
| 2,4-D (.5 mg/L) | 1 | ml |
| Picloram (10 mg/ml) | 200 | μl |
| BAP (1 mg/L) | .5 | ml |

Adjust PH to 5.8 with KOH
Post sterilization add:

| | | |
|---|---|---|
| Acetosyringone (400 μM) | 400 | μl |

TABLE 16

| | Units | |
|---|---|---|
| Medium components | per liter | 13152C |
| MS BASAL SALT MIXTURE | g | 4.3 |
| THIAMINE•HCL | mg | 1.0 |
| L-PROLINE | G | 0.7 |
| CASEIN HYDROLYSATE (ACID) | g | 1.0 |
| MALTOSE | g | 30.0 |
| 2,4-D | mg | 1.0 |
| PHYTAGEL | g | 3.5 |
| MYO-INOSITOL | g | 0.25 |
| CUPRIC SULFATE (100 mM) | ml | 1.22 |
| AGRIBIO Carbenicillin | mg | 100 |
| BAP (1 mg/ml) | mg | 0.5 |
| pH | | 5.8 |

TABLE 17
Media For Soybean Transformation

| | | Shoot | | |
|---|---|---|---|---|
| Medium components | Infection | Induction (SI) | Rooting | |
| Gamborg B5 Basal Medium (g/L) | 0.321 | 3.21 | — | |
| (Phytotech G398*) | | | | |
| MS Modified Basal Medium with | — | — | 2.22 | |
| Gamborg Vitamins (g/L) (Phytotech | | | | |
| M404*) | | | | |
| Phytotech R7100* | | 4.05 | | |
| Sucrose (g/L) (Phytotech S391*) | 30 | 30 | 20 | |
| MES (g/L) | 4.26 | | 0.64 | |
| pH | 5.4 | 5.6 to 5.7 | 5.6 | |
| TC agar (g/L) (Phytotech A175*) | — | 6 | 6 | |
| IBA | — | — | 1 | mg/L |

| GA3 (Phytotech G358*) | 0.25 | mg/L | — | | — |
|---|---|---|---|---|---|
| Zeatin-Riboside | — | | — | | — |
| BAP (Sigma B3274) stock 1 mg/ml | 1.67 | mg/L | 1.11 | mg/L | — |

| | | | | |
|---|---|---|---|---|
| Dithiothrietol (DTT, Phytotech | 1 | ml/L | — | — |
| D259*, stock 1M, final 1 mM) | | | | |
| Acetosyringone (Aldrich D13, 440-6) | 0.2 | ml/L | — | — |
| stock 1M | | | | |
| (final 200 μM) | | | | |

| | | | | | |
|---|---|---|---|---|---|
| Cefotaxime (GoldBio 64485-93-4, | — | 300 | mg/L | 300 | mg/L |
| 94.2%, stock 150 mg/ml) | | | | | |
| Spectinomycin (PhytoTech S742*, | | 0.25 | ml/L | 0.1 | ml/L |
| stock 100 mg/ml) | | | | | |

*PhytoTechnology Laboratories, P.O. Box 12205; Shawnee Mission, KS 66282-2205

TABLE 18
Media For Cowpea Transformation

Infection Medium: MS salts and vitamins, 20 mM MES buffer, 30 g/l sucrose, 0.5 mg/l BAP,
0.5 mg/l kinetin, 0.25 mg/l GA3, 200 mM acetosyringone, 400 mg/l cycteine, 100 mM BCDA
(Bathocuproinedisulfonic acid disodium salt), 50 mg/l thymidine, 1 mg/l polyvinylpyrrolidone,
pH 5.4.
Co-cultivation medium: MS salts and vitamins, 20 mM MES buffer, 30 g/l sucrose, 0.5 mg/l
BAP, 0.5 mg/l kinetin, 0.25 mg/l GA3, 200 mM acetosyringone, 0.5 mM dithiothreitol, 400
mg/l cycteine, 100 mM BCDA (Bathocuproinedisulfonic acid disodium salt), 50 mg/l
thymidine, 1 mg/l polyvinylpyrrolidone, 8 g/l Difco Agar, pH 5.4.
Shoot Initiation Medium: MS salts and vitamins, 3 mM MES buffer, 30 g/l sucrose, 0.5 mg/l
BAP, 0.5 mg/l kinetin, 25 mg/l spectinomycin, 100 mg/l carbenicillin, 2 mg/l silver nitrate, 1
mg/l polyvinylpyrrolidone, 8 g/l Difco Agar, pH 5.6.
Shoot Elongation Medium: MS salts and vitamins, 3 mM MES buffer, 30 g/l sucrose, 0.1 mg/l
kinetin, 0.5 mg/l GA3, 50 mg/l asparagine, 25 mg/l spectinomycin, pH 5.6.
Rooting Medium: MS salts and vitamins, 3 mM MES buffer, 30 g/l sucrose, 0.1 mg/l IBA, 1
mg/l polyvinylpyrrolidone, 2 mg/l silver nitrate, 25 mg/l spectinomycin, pH 5.6.

### Example 23: Sequence Identification

Various sequences are referenced in the disclosure. Sequence identifiers are provided in Table 1 and in Table 19.

TABLE 19

| SEQ | | | |
|---|---|---|---|
| ID | | | |
| NO: | TYPE* | NAME | DESCRIPTION |
| 1 | DNA | PHP71539 | Synthetic construct containing Agrobacterium tumelaciens VIR genes |
| 2 | DNA | PHP89401 | Synthetic construct comprising the T-DNA (RB to LB): RB +LOXP |
| | | | + CCDB + GM-UBQ PRO-V1::GM-UBQ 5′ UTR::GM-UBQ |
| | | | INTRON1::CTP::SPCN::UBQ14 TERM + GM-EF1A2 PRO::GM- |
| | | | EF1A2 5′ UTR::GM-EF1A2 INTRON1::GM-EF1A2 5′ UTR::DS- |
| | | | RED2::UBQ3 TERM +LB |
| 3 | DNA | PHP91619 | Synthetic construct comprising: PRO::UBIlZM 5′ UTR::UBIlZM |
| | | | INTRON1:FRT1:CTP::SPCN::SB-UBI TERM |
| 4 | DNA | PHP75799 | Synthetic construct comprising: ZM-PLTP PRO::ZM-PLTP 5′ |
| | | | UTR::ZM-ODP2::OS-T28 TERM |
| 5 | DNA | PHP76976 | Synthetic construct comprising: ZM-AXIG1 PRO::ZM-WUS2::IN2- |
| | | | 1 TERM |
| 6 | DNA | PHP88871 | Synthetic construct comprising the T-DNA (RB to LB): RB + |
| | | | CAMV35S PRO (PHI)-V5::HA-WUS-V1::OS-T28 TERM (MOD1) |
| | | | + GM-UBQ PRO::GM-UBQ 5′ UTR::GM-UBQ INTRON1::ZS- |
| | | | YELLOW1 N1::NOS TERM + AT-UBIQ10 PRO::AT-UBIQ10 5′ |
| | | | UTR::AT-UBIQ10 INTRON1::CTP::SPCN::UBQ14 TERM + LB |
| 7 | DNA | PHP81356 | Synthetic construct comprising the T-DNA (RB to LB): RB + |
| | | | UBQ14 TERM::SPCN::CTP::AT-UBIQ10 INTRON1::AT-UBIQ10 |
| | | | 5′ UTR::AT-UBIQ10 PRO + UBQ3 TERM::TAGRFP::GM-UBQ |
| | | | INTRON1::GM-UBQ 5′ UTR::GM-UBQ PRO-V1 + LB |
| 8 | DNA | PHP82314 | Synthetic construct comprising the T-DNA (RB to LB): RB + GM- |
| | | | UBQ PRO-V1::GM-UBQ 5′ UTR::GM-UBQ |
| | | | INTRON1::TAGRFP::UBQ3 TERM + AT-UBIQ10 PRO:: AT- |
| | | | UBIQ10 5′ UTR::AT-UBIQ10 INTRON1::CTP::SPCN::UBQ14 |
| | | | TERM + LB |
| 9 | DNA | SPCN | Streptomyces spectabilis SPCN coding sequence |
| 10 | PRT | SPCN | Streptomyces spectabilis SPCN protein sequence |
| 11 | DNA | SPCN | Maize-codon-optimized Streptomyces spectabilis SPCN coding |
| | | (MO1) | sequence |
| 12 | PRT | SPCN | Maize-codon-optimized Streptomyces spectabilis SPCN protein |
| | | (MO1) | sequence |
| 13 | DNA | SPCN | Soybean-codon-optimized SPCN coding sequence |
| | | (SO) | |
| 14 | PRT | SPCN | Soybean-codon-optimized SPCN protein sequence |
| | | (SO) | |
| 15 | DNA | LP-APH | Legionella pheumophila APH coding sequence |
| 16 | PRT | LP-APH | Legionella pheumophila APH protein sequence |
| 17 | DNA | PHP0004 | Synthetic construct comprising: FRT1:PMI:: PINII TERM + |
| | | | UBIlZM PRO::DS- RED2::PINII TERM + FRT87 |
| 18 | DNA | PHP5096 | Synthetic construct comprising: UBIlZM PRO::FLPm::PINII |
| | | | TERM |
| 19 | DNA | PHP89030 | Synthetic construct comprising: ZM-PLTP PRO::ZM-ODP2::0S- |
| | | | T28 TERM + FMV & PCSV ENHANCERS |
| 20 | DNA | PHP89179 | Synthetic construct comprising: ZM-PLTP PRO::ZM-WUS2::IN2-1 |
| | | | TERM |
| 21 | DNA | LP-APH | Maize-codon-optimized LP-APH coding sequence |
| | | (MO1) | |
| 22 | PRT | LP-APH | LP-APH (MO1) encoded protein sequence |
| | | (MO1) | |
| 23 | DNA | LP-APH | Soybean-codon-optimized LP-APH coding sequence |
| | | (SO) | |
| 24 | PRT | LP-APH | LP-APH (SO) encoded protein sequence |
| | | (SO) | |
| 25 | DNA | PHP86170 | Synthetic construct comprising the T-DNA (RB to LB): RB- |
| | | | GMUBQ PRO::GM-UBQ 5UTR::GM-UBQ |
| | | | INTRON1::CTP:SPCN::PINII TERM +DMMV |
| | | | PRO::TAGRFP::UBQ14 TERM + LB |
| 26 | DNA | PHP82311 | Synthetic construct comprising the T-DNA (RB to LB): RB + GM- |
| | | | UBQ PRO-V1::GM-UBQ 5′ UTR::GM-UBQ |
| | | | INTRON1::TAGRFP::UBQ3 TERM + GM-SAMS PRO:: GM- |
| | | | SAMS 5′ UTR:: GM-SAMS INTRON1::CTP::APH::UBQ14 TERM |
| | | | + LB |
| 27 | DNA | PHP82312 | Synthetic construct comprising the T-DNA (RB to LB): RB + GM- |
| | | | UBQ PRO-V1::GM-UBQ 5′ UTR::GM-UBQ |
| | | | INTRON1::TAGRFP::UBQ3 TERM + AT-UBIQ10 PRO:: AT- |
| | | | UBIQ10 5′ UTR:: AT-UBIQ10 INTRON1::CTP::APH::UBQ14 |
| | | | TERM + LB |
| 28 | DNA | PHP82313 | Synthetic construct comprising the T-DNA (RB to LB): RB + GM- |
| | | | UBQ PRO-V1::GM-UBQ 5′ UTR::GM-UBQ |
| | | | INTRON1::TAGRFP::UBQ3 TERM + GM-SAMS PRO:: GM- |
| | | | SAMS 5′ UTR:: GM-SAMS INTRON1::CTP::SPCN::UBQ14 |
| | | | TERM + LB |
| 29 | DNA | PHP81355 | Synthetic construct comprising the T-DNA (RB to LB): RB + |
| | | | UBQ14 TERM::SPCN::CTP::GM-SAMS INTRON1::GM-SAMS 5′ |
| | | | UTR::GM-SAMS PRO + UBQ3 TERM::TAGRFP::GM-UBQ |
| | | | INTRON1::GM-UBQ 5′ UTR::GM-UBQ PRO-V1 + LB |
| 30 | DNA | PHP81354 | Synthetic construct comprising the T-DNA (RB to LB): RB + |
| | | | UBQ14 TERM::APH::CTP::GM-SAMS INTRON1::GM-SAMS 5′ |
| | | | UTR::GM-SAMS PRO + UBQ3 TERM::TAGRFP::GM-UBQ |
| | | | INTRON1::GM-UBQ 5′ UTR::GM-UBQ PRO-V1 + LB |
| 31 | DNA | PHP81359 | Synthetic construct comprising the T-DNA (RB to LB): RB + |
| | | | UBQ14 TERM::APH::CTP:: AT-UBIQ 10 INTRON1:: AT-UBIQ10 |
| | | | 5′ UTR:: AT-UBIQ10 PRO + UBQ3 TERM::TAGRFP::GM-UBQ |
| | | | INTRON1::GM-UBQ 5′ UTR::GM-UBQ PRO-V1 + LB |
| 32 | DNA | PHP92307 | Synthetic construct comprising the T-DNA (RB to LB): RB + LOXP |
| | | | + PLTP:WUS:IN2-1 TERM +ZMHSP17.7:MO-CRE:PINII TERM |
| | | | + UBIlZMPRO:NPTII:SB-UBI TERM + UBIlZM PRO-FRT1 |
| | | | FRT1:CTP::SPCN::SB-UBI TERM + LB) |
| 33 | DNA | PHP1 | Synthetic construct comprising the T-DNA (RB to LB): RB+30LoxP- |
| | | | NOS:WUS:PINII+ZMUBI:ODP2:PINIFRAB17:MOCRE:PINII+ |
| | | | ZMUBI-CTP-SPCN:SB-UBITERM+LB |
| 34 | DNA | PHP2 | Synthetic construct comprising the T-DNA (RB to LB): RB+LoxP- |
| | | | NOS:WUS:PINII+ZMUBI:ODP2:PINIFRAB17:MOCRE:PINII+LB |
| 35 | DNA | PHP3 | Synthetic construct comprising: FRT1:CTP::SPCN::PINII |
| | | | TERM:FRT87 |
| 36 | DNA | PHP4 | Synthetic construct comprising the T-DNA (RB to LB): RB+UBI |
| | | | PRO:UBIlZM INTRON::MO-FLP::PINII TERM+CaMV35S |
| | | | TERM+FRT1:CTP::SPCN::PINII TERM:FRT87+UBI |
| | | | PRO::UBIlZM INTRON::DsRED+30NOS PRO::ZM-WUS2::PINII |
| | | | TERM+UBI PRO:UBIlZM INTRON::ZM-ODP2:: PINII |
| | | | TERM+LB |
| 37 | DNA | PHP49452 | Synthetic construct comprising the T-DNA (RB to LB): RB+GM- |
| | | | SAMS PRO-GM-SAMS UTR-GM SATMS INTRON1-GM-SAMS |
| | | | UTR2-FRT1:CAMV35S PRO:HYG:NOS TERM + GM-UBQ PRO- |
| | | | GM-UBQ 5UTR:ZS-YELLOW:NOS TERM-FRT:87+LB |
| 38 | DNA | PHP92521 | Synthetic construct comprising the T-DNA (RB to LB): RB+AT- |
| | | | UBIQ10 PRO:FLP:UBQ3TERM+FRT1-CTP-SPCN:UBQ10 |
| | | | TERM++GM-MYH11:DS-RED:PINII-FRT87+LB |
| 39 | DNA | PHP92985 | Synthetic construct comprising the T-DNA (RB to LB): RB+GM- |
| | | | EF1A2PRO:FLP:UBQ10 TERM+FRT1-CTP-SPCN:UBQ3 |
| | | | TERM++GM-MYH11PRO:DS-RED:PINII-FRT87+LB |
| 40 | DNA | PHP93448 | Synthetic construct comprising the T-DNA (RB to LB): |
| | | | RB+GMEF1A2 PRO:FLP:UBQ3 TERM+FRT1-CTP-SPCN:UBQ10 |
| | | | TERM+FMVENH+PCSV EHN+MMV ENH+GM-MTH1:DS- |
| | | | RED:PINII-FRT87+LB |
| 41 | DNA | PHP92349 | Synthetic construct comprising the T-DNA (RB to LB): |
| | | | RB+LOXP+GM-QBU PRO::CTP:SPCN::UBQ14 TERM+GM- |
| | | | EF1A2 PRO::DS-RED2::UBQ3 TERM+LOXP+LB |
| 42 | DNA | PHP81184 | Synthetic construct containing Agrobacterium rhizogenes VIR genes |
| 43 | DNA | PHP70298 | Synthetic construct containing Agrobacterium tumefaciens VIR genes |
| 44 | DNA | PHP79761 | Synthetic construct containing Agrobacterium tumefaciens VIR genes |
| 45 | DNA | FRT | GAAGTTCCTATTCTCTAGAAAGTATAGGAACTTC |

* “DNA” indicates a polynucleotide or nucleic acid sequence;
“PRT” indicates a polypeptide or protein sequence.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

1. A plant transformed with a recombinant expression cassette comprising a marker gene cassette comprising a DNA sequence imparting spectinomycin resistance or streptomycin resistance in plants, wherein the DNA sequence comprises a nucleotide sequence selected from the group consisting of:
(a) at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants;
(b) a nucleotide sequence that is at least 95% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants;
(c) a nucleotide sequence that is at least 70% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants;
(d) a nucleotide sequence encoding a polypeptide of at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants;
(e) a nucleotide sequence encoding a polypeptide that is at least 95% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; and
(f) a nucleotide sequence encoding a polypeptide that is at least 70% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants.

2. The plant of claim 1, wherein the recombinant expression cassette further comprises a trait gene cassette comprising a heterologous nucleotide sequence of interest.

3. The plant of claim 2, wherein the heterologous nucleotide sequence of interest comprises a trait gene encoding a gene product conferring nutritional enhancement, pest resistance, herbicide resistance, abiotic stress tolerance, increased yield, drought resistance, cold tolerance, pathogen resistance, insect resistance, nitrogen use efficiency (NUE), disease resistance, or an ability to alter a metabolic pathway.

4. The plant of claim 2, wherein the recombinant expression cassette further comprises a morphogenic gene cassette comprising a morphogenic gene.

5. The plant of claim 4, wherein the morphogenic gene comprises:
(i) a nucleotide sequence encoding a WUS/WOX homeobox polypeptide; or
(ii) a nucleotide sequence encoding a Babyboom (BBM) polypeptide or an Ovule Development Protein 2 (ODP2) polypeptide; or
(iii) a combination of (i) and (ii).

6. The plant of claim 5, wherein the morphogenic gene comprises the nucleotide sequence encoding the WUS/WOX homeobox polypeptide.

7. The plant of claim 6, wherein the nucleotide sequence encoding the WUS/WOX homeobox polypeptide is selected from WUS1, WUS2, WUS3, WOX2A, WOX4, WOX5, and WOX9.

8. The plant of claim 5, wherein the morphogenic gene comprises a nucleotide sequence encoding the Babyboom (BBM) polypeptide or the Ovule Development Protein 2 (ODP2) polypeptide.

9. The plant of claim 8, wherein the nucleotide sequence encoding the Babyboom (BBM) polypeptide is selected from BBM2, BMN2, and BMN3 or the Ovule Development Protein 2 (ODP2) polypeptide is ODP2.

10. The plant of claim 5, wherein the morphogenic gene comprises a nucleotide sequence encoding the WUS/WOX homeobox polypeptide and the Babyboom (BBM) polypeptide or the Ovule Development Protein 2 (ODP2) polypeptide.

11. The plant of claim 10, wherein the nucleotide sequence encoding the WUS/WOX homeobox polypeptide is selected from WUS1, WUS2, WUS3, WOX2A, WOX4, WOX5, and WOX9 and the Babyboom (BBM) polypeptide is selected from BBM2, BMN2, and BMN3 or the Ovule Development Protein 2 (ODP2) polypeptide is ODP2.

12. The plant of claim 4, wherein the recombinant expression cassette further comprises a site-specific recombinase cassette comprising a nucleotide sequence encoding a site-specific recombinase selected from FLP, Cre, SSV1, lambda Int, phi C31 Int, HK022, R, Gin, Tn1721, CinH, ParA, Tn5053, Bxbl, TP907-1, or U153.

13. The plant of claim 12, wherein the site-specific recombinase is operably linked to a constitutive promoter, an inducible promoter, or a developmentally regulated promoter.

14. The plant of claim 13, wherein the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2);
the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and
the developmentally regulated promoter is selected from PLTP, PLTP1, PLTP2, PLTP3, SDR, LGL, LEA-14A, or LEA-D34.

15. The plant of claim 4, wherein the morphogenic gene is operably linked to a constitutive promoter, an inducible promoter, or a developmentally regulated promoter.

16. The plant cell of claim 15, wherein the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2);
the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and
the developmentally regulated promoter is selected from PLTP, PLTP1, PLTP2, PLTP3, SDR, LGL, LEA-14A, or LEA-D34.

17. The plant of claim 1, wherein the plant is a monocot or a dicot.

18. A seed from the plant of claim 2, wherein the seed comprises the trait gene cassette and not the marker gene cassette.

19. A seed from the plant of claim 4, wherein the seed comprises the trait gene cassette and not the marker gene cassette or the morphogenic gene cassette.

20. A seed from the plant of claim 12, wherein the seed comprises the trait gene cassette and not the marker gene cassette or the morphogenic gene cassette or the site-specific recombinase cassette.

21. A method of producing a transgenic plant expressing a trait gene cassette comprising:
transforming a plant cell with a recombinant expression cassette comprising a trait gene cassette and a marker gene cassette, the marker gene cassette comprising a DNA sequence imparting spectinomycin resistance or streptomycin resistance in plants, wherein the DNA sequence comprises a nucleotide sequence selected from the group consisting of:
(a) at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants;
(b) a nucleotide sequence that is at least 95% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants;
(c) a nucleotide sequence that is at least 70% identical to at least one of SEQ ID NO: 9, SEQ ID NO: 11, SEQ ID NO: 13, SEQ ID NO: 15, SEQ ID NO: 21 and SEQ ID NO: 23, wherein the nucleotide sequence imparts spectinomycin resistance or streptomycin resistance in plants;
(d) a nucleotide sequence encoding a polypeptide of at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants;
(e) a nucleotide sequence encoding a polypeptide that is at least 95% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants; and
(f) a nucleotide sequence encoding a polypeptide that is at least 70% identical to at least one of SEQ ID NO: 10, SEQ ID NO: 12, SEQ ID NO: 14, SEQ ID NO: 16, SEQ ID NO: 22, and SEQ ID NO: 24, wherein the nucleotide sequence imparts spectinomycin resistance or streptomycin resistance in plants;
selecting a spectinomycin resistant or a streptomycin resistant transgenic plant cell; and
regenerating the transgenic plant expressing the trait gene cassette.

22. The method of claim 21, wherein the trait gene cassette comprises a heterologous nucleotide sequence of interest.

23. The method of claim 22, wherein the heterologous nucleotide sequence of interest comprises a trait gene encoding a gene product conferring nutritional enhancement, pest resistance, herbicide resistance, abiotic stress tolerance, increased yield, drought resistance, cold tolerance, pathogen resistance, insect resistance, nitrogen use efficiency (NUE), disease resistance, or an ability to alter a metabolic pathway.

24. The method of claim 21, wherein the recombinant expression cassette further comprises a morphogenic gene cassette.

25. The method of claim 24, wherein the morphogenic gene cassette comprises a morphogenic gene.

26. The method of claim 25, wherein the morphogenic gene comprises:
(i) a nucleotide sequence encoding a WUS/WOX homeobox polypeptide; or
(ii) a nucleotide sequence encoding a Babyboom (BBM) polypeptide or an Ovule Development Protein 2 (ODP2) polypeptide; or
(iii) a combination of (i) and (ii).

27. The method of claim 26, wherein the morphogenic gene comprises a nucleotide sequence encoding the WUS/WOX homeobox polypeptide.

28. The method of claim 27, wherein the nucleotide sequence encoding the WUS/WOX homeobox polypeptide is selected from WUS1, WUS2, WUS3, WOX2A, WOX4, WOX5, and WOX9.

29. The method of claim 26, wherein the morphogenic gene comprises a nucleotide sequence encoding the Babyboom (BBM) polypeptide or the Ovule Development Protein 2 (ODP2) polypeptide.

30. The method of claim 29, wherein the nucleotide sequence encoding the Babyboom (BBM) polypeptide is selected from BBM2, BMN2, and BMN3 or the Ovule Development Protein 2 (ODP2) polypeptide is ODP2.

31. The method of claim 26, wherein the morphogenic gene comprises a nucleotide sequence encoding the WUS/WOX homeobox polypeptide and the Babyboom (BBM) polypeptide or the Ovule Development Protein 2 (ODP2) polypeptide.

32. The method of claim 31, wherein the nucleotide sequence encoding the WUS/WOX homeobox polypeptide is selected from WUS1, WUS2, WUS3, WOX2A, WOX4, WOX5, and WOX9 and the Babyboom (BBM) polypeptide is selected from BBM2, BMN2, and BMN3 or the Ovule Development Protein 2 (ODP2) polypeptide is ODP2.

33. The method of claim 24, wherein the recombinant expression cassette further comprises a site-specific recombinase cassette.

34. The method of claim 33, wherein the site-specific recombinase cassette comprises a nucleotide sequence encoding a site-specific recombinase selected from FLP, Cre, SSV1, lambda Int, phi C31 Int, HK022, R, Gin, Tn1721, CinH, ParA, Tn5053, Bxb1, TP907-1, or U153.

35. The method of claim 34, wherein the nucleotide sequence encoding the site-specific recombinase is operably linked to a constitutive promoter, an inducible promoter, or a developmentally regulated promoter.

36. The method of claim 35, wherein the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2);
the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and
the developmentally regulated promoter is selected from PLTP, PLTP1, PLTP2, PLTP3, SDR, LGL, LEA-14A, or LEA-D34.

37. The method of claim 25, wherein the morphogenic gene is operably linked to a constitutive promoter, an inducible promoter, or a developmentally regulated promoter.

38. The method of claim 37, wherein the constitutive promoter is selected from UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, or ZM-ADF PRO (ALT2);
the inducible promoter is selected from AXIG1, DR5, XVE, GLB1, OLE, LTP2, HSP17.7, HSP26, HSP18A, AT-HSP811, AT-HSP811L, GM-HSP173B or promoters activated by tetracycline, ethamethsulfuron or chlorsulfuron; and
the developmentally regulated promoter is selected from PLTP, PLTP1, PLTP2, PLTP3, SDR, LGL, LEA-14A, or LEA-D34.

39. The method of claim 21, further comprising excising or segregating away the marker gene cassette from the transgenic plant expressing the trait gene cassette.

40. The method of claim 24, further comprising excising or segregating away the marker gene cassette and the morphogenic gene cassette from the transgenic plant expressing the trait gene cassette.

41. The method of claim 33, further comprising excising or segregating away the marker gene cassette, the morphogenic gene cassette, and the site-specific recombinase cassette from the transgenic plant expressing the trait gene cassette.

42. The method of claim 21, wherein the plant cell is a monocot or a dicot.

43. A seed from the transgenic plant of claim 21.

44. A seed of the transgenic plant produced by the method of claim 21, wherein the seed comprises the trait gene cassette and not the marker gene cassette.

45. A seed of the transgenic plant produced by the method of claim 24, wherein the seed comprises the trait gene cassette and not the marker gene cassette or the morphogenic gene cassette.

46. A seed of the transgenic plant produced by the method of claim 33, wherein the seed comprises the trait gene cassette and not the marker gene cassette or the morphogenic gene cassette or the site-specific recombinase cassette.

47. The method of any one of claim 21, wherein the recombinant expression cassette resides in a disarmed Agrobacteria, an Ochrobactrum bacteria or a Rhizobiaceae bacteria.

48. The method of claim 47, wherein the disarmed Agrobacteria is selected from the group of AGL-1, EHA105, GV3101, LBA4404, and LBA4404 THY-.

49. The method of claim 47, wherein the Ochrobactrum bacteria is selected from Table 2.

50. The method of claim 47, wherein the Rhizobiaceae bacteria is selected from Table 3.

51. The method of claim 24, wherein the recombinant expression cassette resides in a disarmed Agrobacteria, an Ochrobactrum bacteria or a Rhizobiaceae bacteria.

52. The method of 33, wherein the recombinant expression cassette resides in a disarmed Agrobacteria, an Ochrobactrum bacteria or a Rhizobiaceae bacteria.
